(19) United States
(12) Patent Application Publication
(10) Pub. No.: US 2021/0292347 A1
(43) Pub. Date: Sep. 23, 2021

(54) DIHETEROCYCLIC TRIAZINONE NUCLEOSIDE ANALOGS AND THEIR USE FOR MEDICATION

(71) Applicant: Lifeng Xu, San Diego, CA (US)

(72) Inventor: Lifeng Xu, San Diego, CA (US)

(21) Appl. No.: 16/805,703

(22) Filed: Feb. 29, 2020

(51) Int. Cl. C07F 9/6561; A61K 31/683; C07D 487/04; A61K 31/53; C07H 19/23; A61K 31/706; A61K 31/675; A61P 35/00

(52) U.S. Cl. CPC C07F 9/6561; A61K 31/683; C07D 487/04; A61P 35/00; C07H 19/23; A61K 31/706; A61K 31/675; A61K 31/53

(57) ABSTRACT

This invention relates to novel of formula I that are useful for the treatment, prevention and/or amelioration of human diseases of cancers.

This invention also relates with their pharmaceutical compositions, preparative General Methods and applications.

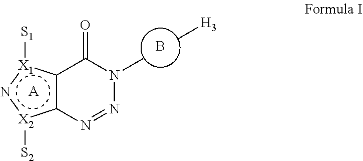

### THE FIELD OF THE INVENTION

This invention relates to novel diheterocyclic triazinone nucleoside analogs that are useful for the treatment, prevention and/or amelioration of human diseases of cancers, the pharmaceutical compositions containing these compounds and the General Methods for their preparation.

### BACKGROUND OF THE INVENTION

Nucleoside analogs have been shown to be potent inhibitors of cancers. There is very few the research on the nucleosides of diheterocyclic triazinone. Only one report is that Manfredini, Stefano; et al.; Journal of Medicinal Chemistry; vol. 35 (1992) p. 917-924 describes the process and activities of the nucleoside, pyrazolo [1,2,3]triazinone with carbonhydrate at 5-position and 6-position to form the nucleoside compounds. To date there has been no report related with glycosylation at 3-position of pyrazolo [1,2,3]triazinone and nor studies of their anticancer activity, nor their structure-activity relationship studies of anticancer activity from all literature reviewed.

### DETAILED DESCRIPTION OF THE INVENTION

The purpose of the present invention is to provide a novel diheterocyclic triazinone nucleoside analogs to their use as anticancer agents, to pharmaceutical compositions containing these compounds and to the General Method s for their preparation, which have the general formula I

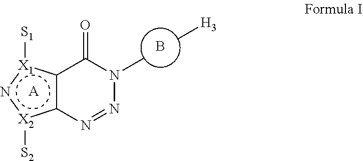

or stereoisomers, tautoers, prodrug, pharmaceutically acceptable salts, complex salts or solvates thereof, wherein: the dotted lines [symbol] are absent or selected but is not limited from: —C—C—, —C═C—, —C-heterobond, —C=heterobond,

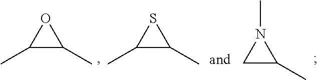

X₁ and X₂ is absent or independently selected but is not limited from: hydrogen, —C—, —S—, —O—, —NH—,

S₁ and S₂ is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, aryl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —C(O)OC₁₋₆alkyl, —C(O)NHC₁₋₆alkyl, P(O)(OH)₂, P(O)(O₁₋₆alkyl)₂, —(CH₂)₁₋₆P(O)(OH)₂, —(CH₂)₁₋₆P(O)(OC₁₋₆alkyl)₂, C₃₋₈amino acid, C₃₋₁₀(OH)₀₋₁₀polyhydroxide;
H₃ is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —(CH₂)₁₋₁₀(OH)₁₋₇, —CH((CH₂)₁₋₁₀(OH)₁₋₇)₁₋₃, —(CH₂)₁₋₁₀(OC(O)C₁₋₆alkyl)₁₋₇, —C(O)NHC₁₋₆alkyl, —P(O)(O₁₋₆alkyl)₂, —(CH₂)₀₋₆P(O)(OH)₂, —(CH₂)₀₋₆P(O)(OC₁₋₆alkyl)₂, —(CH₂)₀₋₆(P(O)(NC₁₋₆alkyl)₂)₁₋₃(OC₁₋₆alkyl)₂)₁₋₃, C₃₋₈amino acid.

A ring of

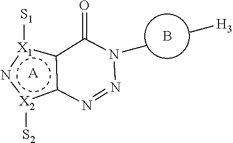

is selected but is not limited from:

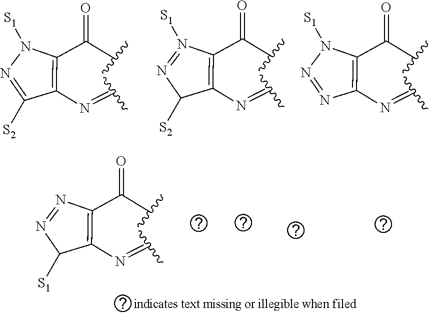

to form

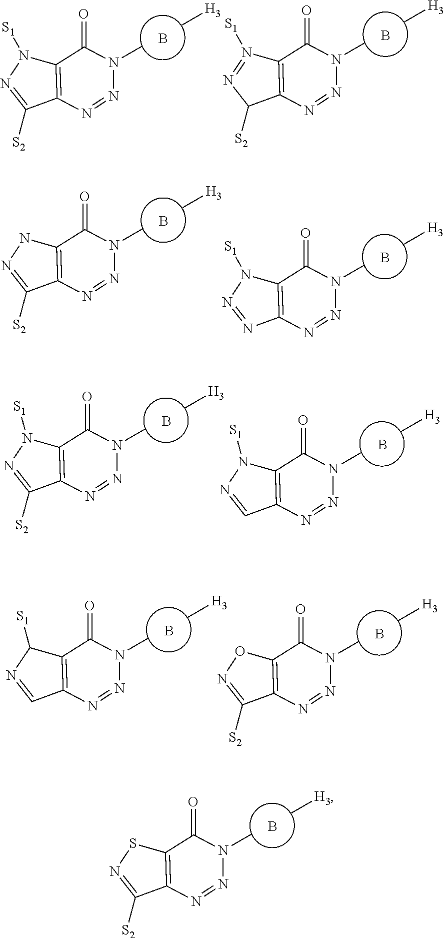

B ring of

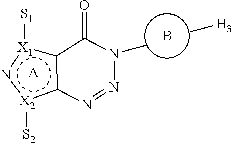

is absent or selected but is not limited from:

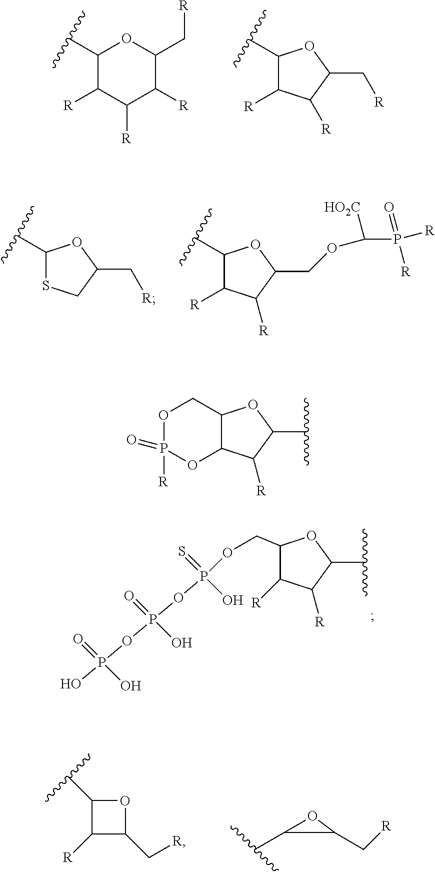

to form

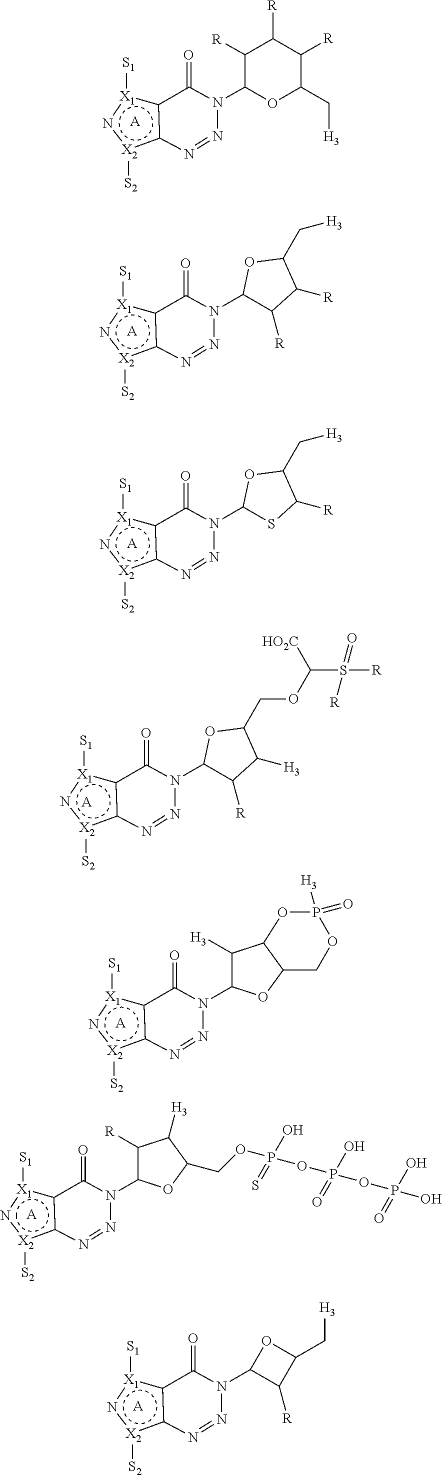

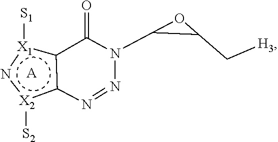

said R is selected but are not limited from hydrogen, halogen, —CN, —CF₃, —OH, —NH₂, guanidine, amidine, —SH, —P(O)(OH)₂, C₃₋₈amino acid, —OC₁₋₆alkyl, —OC₂₋₆alkenyl, —OC₂₋₆alkynyl, —OC₃₋₁₀cycloalkyl, —OC₃₋₁₀cycloalkenyl, O-aryl, —OC₃₋₁₀heterocycle, —OC₃₋₁₀heteroaryl, —NHC₁₋₆alkyl, —NHC₂₋₆alkenyl, —NHC₂₋₆alkynyl, —NHC₃₋₁₀cycloalkyl, —NHC₃₋₁₀cycloalkenyl, NH-aryl, —NHC₃₋₁₀heterocycle, —NH₃₋₁₀heteroaryl, —N(C₁₋₆alkyl)₂, —N(C₂₋₆alkenyl)₂, —N(C₂₋₆alkynyl)₂, —N(C₃₋₁₀cycloalkyl)₂, —N(C₃₋₁₀cycloalkenyl)₂, N(aryl)₂, —N(C₃₋₁₀heterocycle)₂, —N(C₃₋₁₀heteroaryl)₂, —NHSO₂C₁₋₆alkyl, —NHC(O)C₁₋₆alkyl,
—SC₁₋₆alkyl, —SC₂₋₆alkenyl, —SC₂₋₆alkynyl, —SC₃₋₁₀cycloalkyl, —SC₃₋₁₀cycloalkenyl, S-aryl, —SC₃₋₁₀heterocycle, —SC₃₋₁₀heteroaryl, —SOC₁₋₆alkyl, —SO₂C₁₋₆alkyl, —SO₂NHC₁₋₆alkyl, —C₁₋₆alkyl, —C₂₋₆alkenyl, —C₂₋₆alkynyl, —C₃₋₁₀cycloalkyl, —C₃₋₁₀cycloalkenyl, -aryl, —C₃₋₁₀heterocycle, —C₃₋₁₀heteroaryl, —C(O)OH, —C(O)OC₁₋₆alkyl, —C(O)NHC₁₋₆alkyl, —OP(OC₁₋₆alkyl)₂, —OP(OC₂₋₆alkenyl)₂, —OP(OC₂₋₆alkynyl)₂, —OP(OC₃₋₁₀cycloalkyl)₂, —OP(OC₃₋₁₀cycloalkenyl)₂, OP-(Oaryl)₂, —OP(OC₃₋₁₀heterocycle)₂, —OP(OC₃₋₁₀heteroaryl)₂, —NP(O)(NC₁₋₆alkyl)(OC₁₋₆alkyl), —NP(O)(NC₂₋₆alkenyl)(OC₂₋₆alkenyl), —NP(O)(NC₂₋₆alkynyl)(OC₂₋₆alkynyl), —NP(O)(NC₃₋₁₀cycloalkyl)O (OC₃₋₁₀cycloalkyl), —NP(O)(NC₃₋₁₀cycloalkenyl)(OC₃₋₁₀cycloalkenyl), —NP(O)(N-aryl)(O-aryl), —NP(O)(NC₃₋₁₀heterocycle)(OC₃₋₁₀heterocycle), —NP(O)(NC₃₋₁₀heteroaryl)(OC₃₋₁₀heteroaryl), —NP(O)(NC₁₋₆alkyl)₂, —NP(O)(NC₂₋₆alkenyl)₂, —NP(O)(NC₂₋₆alkynyl)₂, —NP(O)(NC₃₋₁₀cycloalkyl)₂, —NP(O)(NC₃₋₁₀cycloalkenyl)₂, —NP(O)(N-aryl)₂, —NP(O)(NC₃₋₁₀heterocycle)₂, —NP(O)(NC₃₋₁₀heteroaryl)₂.

A compound of novel triazinone nucleoside analogs is selected but is not limited from the exemplified examples or stereoisomers, tautomers, pharmaceutically acceptable salts, inorganic acid salt, organic acid salt, inorganic basic salt, organic basic salt, complex salt, prodrug or solvates thereof in association with a pharmaceutically acceptable excipient or carrier; in addition, an acid or a base may be incorporated into the composition to facilitate processing, to enhance stability, or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane and the like; bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, parabromophenylsulfonic acid, propionic acid, p-toluenesulfonic acid, salicylic acid, stearic acid, succinic acid, taimic acid, tartaric acid, thioglycolic acid, toluenesulfonic acid, uric acid, and the like.

A process for the manufacture of a compound of formula I to form novel triazinone nucleoside analogs is obtained by modification of dicyclic triazinone compounds at 3-position with a bond of C—N, under catalysis at −78° C. to 90° C., by a solvent selected from THF, 1, 4-dioxane, N, N-dimethylformamide, N, N-dimethylacetamide, toluene, CH₃CN, CH₂Cl₂, C₆H₆ and a catalyst selected from SnCl₄, NH₃, SnCl₂, SiF₄, Bu₄NF, Hg(CN)₂, HgBr₂, Bu₄NI, Hg(OAc)₂, organic base, inorganic base, molecular sieves or alumina; in addition, the present invention is composed of the base to increases the solubility, to enhance the stability or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl) aminomethane.

A compound of novel triazinone nucleoside analogs is independently at each occurrence, selected but is not limited from the example 1 to 167 and the list of diheterocyclic triazinone nucleoside.

The amount of one or more of the compounds of general formula I, or a pharmaceutically acceptable salt thereof, present in the composition for treating, preventing or slowing the progression of cancer, virus and other diseases, including inflammation, inflammatory diseases and immune system disease associated with cancer, alone or with the following drugs known to be used in conjunction dose in a range of about 0.001 mg-2.0 g/kg; means of various General Method s of treatment and therapy, where the cancers are selected but are not limited from the group consisting of busulfan, cisplatin, mitomycin C, carboplatin, colchichine, vinblastine, paclitaxel, docetaxel, camptochecin, topotecan, doxorubicin, etoposide, 5-azacytidine, 5-fluorouracil, methotrexate, 5-fluoro-2′-deoxy-uridine, ara-C, hydroxylurea, thioguanine, melphalan, chlorambucil, cyclo phosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin, tamsulosin, CB-64D, CB-184, haloperidol, lovastatin, simvastatin, pravastatin, fluvastatin, atorvastatin, cerivastatin, amprenavir, abavavir, indinavir, nelfinavir, tipranavir, ritonavir, saquinavir, bexarotene, tretinoin, 13-cis-retinoic acid, 9-cis-retinoic acid, difluoromethylornithine, fenretinide, N-4-carboxyphenyl retinamide, lactacystin, genistein, flavopiridol, roscovitine, olomoucine, celecoxib, valecoxib, rofecoxib and alanosine, CGP-73547, CGP-61755, DMP-450, ABT-378, AG1776, BMS232,632, ILX23-7553, MG-132, PS341, Gleevec®, ZD1839, SH268, CEP2563, SU6668, SU11248, EMD121974, R115777, SCH66336, L-778,123, BAL9611, TAN-1813 or/and UCN-01.

A compound of novel diheterocyclic triazinone nucleoside analogs is selected for treating, preventing or slowing the progression of the group consisting of Hodgkin's disease, non-Hodgkin's, lymphoma, acute and chronic lymphocytic leukemias, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant mela-noma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogentic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortical carcinoma, skin cancer and prostatic carcinoma.

The administration of a compound of diheterocyclic triazinone nucleoside derivatives and analogs may be by oral route, parenteral, subcutaneous, intravenous, intramuscular, intra-peritoneal, transdermal, buccal, intrathecal, intracranial, intranasal or topical routes.

### Chemical Synthesis

The following reaction schemes illustrate General Methods which may be employed for the synthesis of the compounds of structural formula I described in this invention. All substituents are as defined above unless indicated otherwise. Several strategies based upon synthetic transformations known in the literatures of organic synthesis may be employed for the preparation of the title compounds of general formula I.

In Scheme I, dicyclic triazinone analogs A in hexamethyldisilazane was reacted with acetyled sugar by the reflux in the presence of NH₄SO₄ and SnCl₄ catalyst to afford dicyclic protecting triazinone nucleoside analogs B. In Scheme II, five-member heterocyclic compound C in diluted HCl solution was reacted with NaNO₂ in the presence of base catalyst to afford compound D with the formation of triazinone. In Scheme III, 2-cyanoacetamide E was reacted with an azide reagent to afford substituted triazole carboxamide compound E.

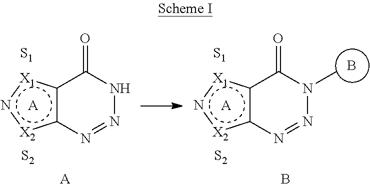

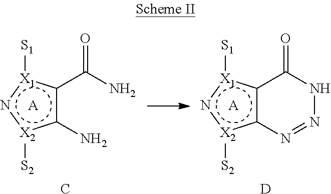

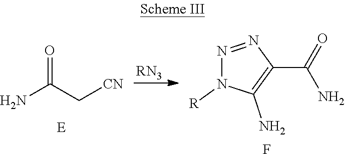

In Scheme IV, compound G was nitrated with nitric acid and then was reducted with H₂/Pd to afford compound H by formation of amino group. In Scheme V, with same General Method to afford compound J by formation of amino group.

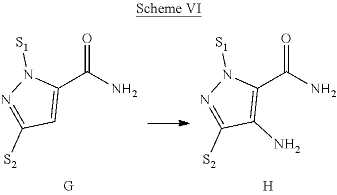

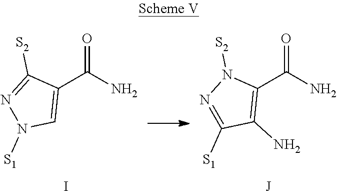

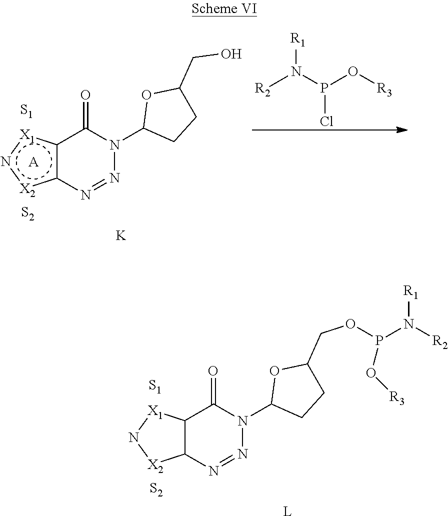

In Scheme VI, compound K was reacted with chlorophosphinamine compound in the presence of base to afford compound L by formation of P—O bond.

### SYNTHESIS AND PREPARATIVE EXAMPLE

### Implementation of the Case and its Structure 1-167 Table 1

### EXAMPLES

The following examples illustrate the present invention. If no mentioned otherwise, the reactions take place at room temperature.

### General Method A (Amination)

General Method I: To a mixture of arylheterocyclic compound (10 mmol) and concentrated sulfuric acid (15 mmol) in THF 20 mL was added concentrated nitric acid (15 mmol). The mixture was stirred until the reaction was complete. The reaction solution was filtered. The crude was separated by silica gel column chromatography to give the nitrated compound.

General Method II: To a mixture of above compound (10 mmol) and C/pd in methanol 20 mL. was added H₂, The mixture was stirred until the reaction was complete. The reaction solution was filtered. The crude was separated by silica gel column chromatography to give the titled compound.

### General Method B (Cyclization)

To a mixture of arylheterocyclic compound with o-aminocarboxamide (10 mmol) and 2N HCl 20 mL was added NaNO₂ (10 mmol). The mixture was refluxed for 2 h. and then was added 2N NaCO₃ to pH˜10. The reaction solution was filtered. The crude was purified by recrystallization to give the title compound.

### General Method C (Glycosidadon)

General Method I: To a mixture of above compound (10 mmol), SnCl₄ (10 mmol), HDMS (10 mmol) and (NH₄)₂SO₄ (10 mmol) in MeOH 20 ml. were added acetylated carbohydrate (10 mmol). The mixture was refluxed for 2 h, The reaction solution was extracted by EtOAc. The crude was separated by silica gel column chromatography to give the protected glycoside compound.

General Method II: To a mixture of above compound (10 mmol), and MeOH 20 mL were added NaOMe (10 mmol). The mixture was stirred for 5 h. and was added acetic acid to pH˜8 The reaction solution was extracted by EtOAC. The crude was separated by silica gel column chromatography to give the titled compound.

### General Method D (Acetylation of Carbohydrate)

To a mixture of carbohydrate compound (10 mmol) and pyrimidy 20 mL were added acetic anhydride (12 mmol). The mixture was refluxed for 4 h. The reaction solution was extracted by EtOAC. The crude was separated by silica gel column chromatography to give the title compound.

### General Method E (Deprotection of Carbohydrate)

To a solution of above protected carbohydrate compound (10 mmol) was added solution of methanol/NaOCH₃ 20 mL (1 mol/L). The mixture was reacted for 5 h and added acetic acid to pH˜7. The reaction solution was extracted by EtOAC. The crude was separated by silica gel column chromatography to give the title compound.

### General Method F (Protection of Premiry-Hydroxy)

To the solution 4,4′-dimethoxytriphenylmethyl chloride (12 mmol) was added the compound of premiry hydroxy (9 mmol) in pyridine 20 mL, and the mixture was stirred for overnight. The crude product was purified by flash chromatography to give the title compound.

### General Method G (Diisopropylphosphoramidition of Secondary Hydroxy)

To a solution of above compound (0.31 mmol) in CH₂Cl₂ (5 mL), 2-cyanoethyl)-N,N-diisopropylchlorophos-phoramidite (0.45 mmol) and diisopropyl ethylamine (0.56 mmol) w as added. The mixture was stirred for 30 min, then diluted with CH₂Cl₂. The reaction solution was washed with 5% aq. NaHCO solution and then the residue was purified by flash chromatography to give the title compound.

### General Method H (Phosphorothioylamidition of Primary Hydroxy)

To a solution of the compound of primary hydroxy (0.41 mmol) in THF 2 mL were added N-methylimidazole (NMI) (0.2 mL) and (2S)-isopropyl 2-((chloro(phenoxy) phosphorothioyl)amino) propanoate (1.2 mmol). The reaction mixture was heated to 70° C. and stirred for 3 hour. The solvents were evaporated and the resulting residue was purified by flash chromatography to give the title compound.

### General Method I (Triphosphate)

To a solution of the nucleoside compound (8.4 mmol) was added triethyl phosphate (15 mL) by heating to 100° C. for 5 minutes. And then Phosphorous oxychloride (1.8 eq., 0.141 mL) was then added to the reaction mixture with stirring. Tributylamine (0.65 mL) was added followed by the addition of anhydrous acetonitrile (10.0 mL), and tributylammonium pyrophosphate (4.0 eq.) was added. The reaction mixture was quenched with 20 mL of 2M TEAB after 15 minutes at 0° C., then stirred overnight. The solvents were evaporated and the resulting residue was purified by flash chromatography to give the title compound.

### General Method J (Cyclization of 5-hydroxy-3H-pyrazolo[4,3-d][1,2,3]triazin-4(5H)-one)

To the solution 2-amino-3-oxosuccinamide (12 mmol) in DMF 25 mL was added hydroxyhydrazine (12 mmol) in DMF 10 mL, and the mixture was stirred at 120° C. for overnight. The crude product was purified by flash chromatography to give the cyclized pyrazolecarboxamide compound for next step. Analogously to General Method B, the title compound was prepared from above 3,4-diamino-1-hydroxy-1H-pyrazole-5-carboxamide and NaNO₂.

### General Method K (Amidation)

To a mixture of amine compound (10 mmol) and pyrimidy 20 mL were added acylchloride compound (12 mmol). The mixture was refluxed for 4 h. The reaction solution was extracted by EtOAC. The crude was separated by silica gel column chromatography to give the title compound.

### Example 1. Preparation of 2-(diethoxyphosphoryl)-2-((5-hydroxy-3-(3-(hydroxymethyl)oxiran-2-yl)-4-oxo-4,5-dihydro-3H-pyrazolo[4,3-d][1,2,3]triazin-7-yl)amino) acetic acid

Analogously to General Method C, the 2-(diethoxyphosphoryl)-2-(1-(3-(hydroxymethyl) oxiran-2-yl)-5,6-dioxo-1,4,5,6-tetrahydro-1,2,3-triazine-4-carboxamido)acetic acid was prepared from 2-(diethoxyphosphoryl)-2-(5,6-dioxo-1,4,5,6-tetrahydro-1,2,3-triazine-4-carboxamido)acetic acid and 3-(acetoxymethyl)oxiran-2-yl acetate; Analogously to General Method J and General Method E, the title compound was prepared from 2-(diethoxy-phosphoryl)-2-(1-(3-(hydroxymethyl) oxiran-2-yl)-5,6-dioxo-1,4,5,6-tetrahydro-1,2,3-triazine-4-carboxamido)acetic acid and hydroxyhydrazine. ¹H NMR (600 MHZ, CDCl₃-d₆): δ 11.00 (s, 1H), 4.63 (d, J=7.0 Hz, 1H), 4.07 (m, 4H), 4.00 (s, 1H), 3.66 (m, 2H), 3.65 (s, 1H), 3.50 (d, J=11.5 Hz, 1H), 3.24 (m, J=7.0 Hz, 1H), 2.00 (s, 1H), 1.29 (t, 6H).

### Example 2. Preparation of ethyl 7-hydroxy-2-(3-(hydroxymethyl)oxiran-2-yl)-1-oxo-2, 7-dihydro-1H-pyrazolo[4,3-d][1,2,3]triazine-5-carboxylate

Analogously to General Method C, ethyl ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 5,6-dioxo-1,4,5,6-tetrahydro-1,2,3-triazine-4-carboxylate and 3-(acetoxymethyl)oxiran-2-yl acetate; Analogously to General Method J and General Method E, the title compound was prepared from ethyl 2-(1-(3-(hydroxymethyl)oxiran-2-yl)-5,6-dioxo-1,4,5,6-tetrahydro-1,2,3-triazin-4-yl)-2-Oxoacetate and hydroxyhydrazine. ¹H NMR (600 MHZ, CDCl₃-d₆): δ 4.63 (d, J=7.0 Hz, 1H), 4.30 (m, J=8.0 Hz, 2H), 3.66 (m, 2H), 3.65 (s, 1H), 3.24 (m, J=7.0 Hz, 1H), 2.00 (s, 1H), 1.29 (t, J=8.0 Hz, 3H).

### Example 3. Preparation of N-(5-hydroxy-3-(3-(hydroxymethyl)oxiran-2-yl)-4-oxo-4,5-dihydro-3H-pyrazolo [4,3-d][1,2,3]triazin-7-yl)-4-methoxybenzamide

Analogously to General Method C, 1-(3-(hydroxymethyl)oxiran-2-yl)-N-(4-methoxybenzoyl)-5,6-dioxo-1,4,5,6-tetrahydro-1,2,3-triazine-4-carboxamide was prepared from N-(4-methoxybenzoyl)-5,6-dioxo-1,4,5,6-tetrahydro-1,2,3-triazine-4-carboxamide and 3-(acetoxymethyl)oxiran-2-yl acetate; Analogously to General Method J and General Method E, the title compound was prepared from 1-(3-(hydroxymethyl)oxiran-2-yl)-N-(4-methoxybenzoyl)-5,6-dioxo-1,4,5,6-tetrahydro-1,2,3-triazine-4-carboxamide and hydroxyhydrazine. ¹H NMR (600 MHZ, CDCl₃-d₆): δ 9.15 (s, 1H), 7.92 (d, 2H), 7.17 (d, 2H), 4.63 (d, J=7.0 Hz, 1H), 3.83 (s, 3H), 3.66 (m, 2H), 3.65 (s, 1H), 3.24 (m, J=7.0 Hz, 1H), 2.00 (s, 1H).

### Example 4. Preparation of 3-(dimethylamino)-N-(5-hydroxy-3-(3-(hydroxymethyl) oxiran-2-yl)-4-oxo-4,5-dihydro-3H-pyrazolo[4,3-d][1,2,3]triazin-7-yl)propanamide

Analogously to General Method C, N-(3-(dimethylamino)propanoyl)-1-(3-(hydroxymethyl)oxiran-2-yl)-5,6-dioxo-1,4,5,6-tetrahydro-1,2,3-triazine-4-carboxamide was prepared from N-(3-(dimethylamino)propanoyl)-5,6-dioxo-1,4,5,6-tetrahydro-1,2,3-triazine-4-carboxamide and 3-(acetoxymethyl)oxiran-2-yl acetate; Analogously to General Method J and General Method E the title compound was prepared from N-(3-(dimethylamino)propanoyl)-5,6-dioxo-1,4,5,6-tetrahydro-1,2,3-triazine-4-carboxamide and hydroxyhydrazine. ¹H NMR (600 MHZ, CDCl₃-d₆): δ 9.15 (s, 1H), 4.63 (d, J=7.0 Hz, 1H), 3.66 (m, 2H), 3.65 (t, J=7.1 Hz, 2H), 3.60 (s, 1H), 3.24 (m, J=7.0 Hz, 1H), 2.84 (s, 6H), 2.50 (t, J=7.1 Hz, 2H), 2.00 (s, 1H).

### Example 5. Preparation of 7-hydroxy-2-(3-(hydroxymethyl)oxiran-2-yl)-5-(piperidin-1-yl)-2,7-dihydro-1H-pyrazolo[4,3-d][1,2,3]triazin-1-one

Analogously to General Method C, 3-(3-(hydroxymethyl)oxiran-2-yl)-6-(piperidine-1-carbonyl)-1,2,3-triazine-4,5(3H,6H)-dione was prepared from -(piperidine-1-carbonyl)-1,2,3-triazine-4,5(3H,6H)-dione and 3-(acetoxymethyl)oxiran-2-yl acetate; Analogously to General Method J and General Method E the title compound was prepared from 3-(-3-(hydroxymethyl) oxiran-2-yl)-6-(piperidine-1-carbonyl)-1,2,3-triazine-4,5(3H,6H)-dione and hydroxyhydrazine. ¹H NMR (600 MHZ, CDCl₃-d₆): δ 4.63 (d, J=7.0 Hz, 1H), 3.66 (m, 2H), 3.65 (s, 1H), 3.24 (m, J=7.0 Hz, 1H), 3.11 (s, 4H), 2.00 (s, 1H), 1.65 (d, 6H).

### Example 6. Preparation of 7-hydroxy-2-(3-(hydroxymethyl)oxiran-2-yl)-5-nitro-2,7-dihydro-1H-pyrazolo[4,3-d][1,2,3]triazin-1-one

Analogously to General Method C, 3-(3-(hydroxymethyl)oxiran-2-yl)-6-(nitrocarbonyl)-1,2,3-triazine-4,5(3H,6H)-dione was prepared from 6-(nitrocarbonyl)-1,2,3-triazine-4,5(3H,6H)-dione and 3-(acetoxymethyl)oxiran-2-yl acetate; Analogously to General Method J and General Method E the title compound was prepared from 3-(3-(hydroxymethyl)oxiran-2-yl)-6-(nitrocarbonyl)-1,2,3-triazine-4,5(3H,6H)-dione and hydroxyhydrazine. ¹H NMR (600 MHZ, CDCl₃-d₆): δ 4.63 (d, J=7.0 Hz, 1H), 3.66 (m, 2H), 3.65 (s, 1H), 3.24 (m, J=7.0 Hz, 1H), 2.00 (s, 1H).

### Example 7. Preparation of ethyl 7-(hydroxymethyl)-2-(3-(hydroxymethyl) oxiran-2-yl)-1-oxo-2,7-dihydro-1H-pyrazolo[4,3-d][1,2,3]triazine-5-carboxylate

Analogously to General Method C, ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 3-(acetoxymethyl)oxiran-2-yl acetate; Analogously to General Method J and General Method E the title compound was prepared from ethyl 2-(1-(3-(hydroxymethyl)oxiran-2-yl)-5,6-dioxo-1,4,5,6-tetrahydro-1,2,3-triazin-4-yl)-2-oxoacetate and hydrazinylmethanol. ¹H NMR (600 MHZ, CDCl₃-d₆): δ 5.97 (s, 2H), 4.63 (d, J=7.0 Hz, 1H), 4.30 (m, J=8.0 Hz, 2H), 3.66 (m, 2H), 3.65 (s, 2H), 3.24 (m, J=7.0 Hz, 1H), 1.29 (t, J=8.0 Hz, 3H).

### Example 8. Preparation of ethyl 7-(2-amino-2-oxoethyl)-2-(3-(hydroxymethyl)oxiran-2-yl)-1-oxo-2,7-dihydro-1H-pyrazolo[4,3-d][1,2,3]triazine-5-carboxylate

Analogously to General Method C, ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 3-(acetoxymethyl)oxiran-2-yl acetate; Analogously to General Method J and General Method E the title compound was prepared from ethyl 2-(1-(3-(hydroxymethyl)oxiran-2-yl)-5,6-dioxo-1,4,5,6-tetrahydro-1,2,3-triazin-4-yl)-2-oxoacetate and 2-hydrazinylacetamide. ¹H NMR (600 MHZ, CDCl₃-d₆): δ 7.16 (s, 2H), 5.60 (s, 2H), 4.63 (d, J=7.0 Hz, 1H), 4.30 (m, J=8.0 Hz, 2H), 3.66 (m, 2H), 3.65 (s, 1H), 3.24 (m, J=7.0 Hz, 1H), 1.29 (t, J=8.0 Hz, 3H).

### Example 9. Preparation of ethyl 7-cyclopropyl-2-(3-(hydroxymethyl)oxiran-2-yl)-1-oxo-2,7-dihydro-1H-pyrazolo[4,3-d][1,2,3]triazine-5-carboxylate

Analogously to General Method C, ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 3-(acetoxymethyl) oxiran-2-yl acetate; Analogously to General Method J and General Method E the title compound was prepared from ethyl 2-(1-(3-(hydroxymethyl)oxiran-2-yl)-5,6-dioxo-1,4,5,6-tetrahydro-1,2,3-triazin-4-yl)-2-oxoacetate and cyclopropylhydrazine. ¹H NMR (600 MHZ, CDCl₃-d₆): δ 4.63 (d, J=7.0 Hz, 1H), 4.30 (m, J=8.0 Hz, 2H), 3.66 (m, 2H), 3.65 (s, 1H), 3.24 (m, J=7.0 Hz, 1H), 2.42 (m, J=7.0 Hz, 1H), 1.29 (t, J=8.0 Hz, 3H), 0.66 (m, 4H).

### Example 10. Preparation of ethyl 7-carbamoyl-2-(3-(hydroxymethyl)oxiran-2-yl)-1-oxo-2,7-dihydro-1H-pyrazolo[4,3-d][1,2,3]triazine-5-carboxylate

Analogously to General Method C, ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 3-(acetoxymethyl)oxiran-2-yl acetate; Analogously to General Method J and General Method E the title compound was prepared from ethyl 2-(1-(3-(hydroxymethyl)oxiran-2-yl)-5,6-dioxo-1,4,5,6-tetrahydro-1,2,3-triazin-4-yl)-2-oxoacetate and hydrazinecarboxamide. ¹H NMR (600 MHZ, CDCl₃-d₆): δ 7.68 (s, 2H), 4.63 (d, J=7.0 Hz, 1H), 4.30 (m, J=8.0 Hz, 2H-), 3.66 (m, 2H), 3.65 (s, 1H), 3.24 (m, J=7.0 Hz, 1H), 1.29 (t, J=8.0 Hz, 3H).

### Example 11. Preparation of 2-(diethoxyphosphoryl)-2-(7-(ethoxycarbonyl)-3-(3-(hydroxymethyl)oxiran-2-yl)-4-oxo-3H-pyrazolo[4,3-d][1,2,3]triazin-5(4H)-yl)acetic acid

Analogously to General Method C, ethyl 2-(1-(3-(hydroxymethyl)oxiran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 3-(acetoxymethyl)oxiran-2-yl acetate; Analogously to General Method J and General Method E the title compound was prepared from ethyl 2-(1-(3-(hydroxymethyl)oxiran-2-yl)-5,6-dioxo-1,4,5,6-tetrahydro-1,2,3-triazin-4-yl)-2-oxoacetate and 2-(diethoxyphosphoryl)-2-hydrazinylacetic acid. ¹H NMR (600 MHZ, CDCl₃-d₆): δ 11.00 (s, 1H), 4.60 (d, J=11.5 Hz, 1H), 4.63 (d, J=7.0 Hz, 1H), 4.30 (m, J=8.0 Hz, 2H), 4.07 (m, 4H), 3.66 (m, 2H), 3.65 (s, 1H), 3.24 (m, J=7.0 Hz, 1H), 1.29 (t, 9H).

### Example 12. Preparation of 2-(diethoxyphosphoryl)-2-((3-(3,4-dihydroxy-5-(hydroxymethyl)tetrahydrofuran-2-yl)-5-hydroxy-4-oxo-4,5-dihydro-3H-pyrazolo[4,3-d][1,2,3]triazin-7-yl)amino) acetic acid

Analogously to General Method C, the intermediate compound (I) 2-(acetoxymethyl)-5-(4-(2-ethoxy-2-oxoacetyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-oxoacetate and 5-(acetoxymethyl)tetrahydrofuran-2,3,4-triyl triacetate for next step. Analogously to General Method J, the intermediate compound (II) 2-(acetoxymethyl)-5-(7-(ethoxycarbonyl)-5-hydroxy-4-oxo-4,5-dihydro-3H-pyrazolo[4,3-d][1,2,3]triazin-3-yl)tetrahy-drofuran-3,4-diyl diacetate was prepared from above intermediate compound (I) and hydroxyhydrazine for next step. Analogously to General Method E, the title compound was prepared from the intermediate compound (II) 6.34 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ11.00 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 4.07 (m, 4H), 4.00 (s, 1H), 3.66 (m, 2H), 3.65 (s, 1H), 3.58 (s, 2H), 3.50 (d, J=11.5 Hz, 1H), 2.00 (s, 1H), 1.29 (t, 6H).

### Example 13. Preparation of ethyl 3-(3,4-dihydroxy-5-(hydroxymethyl) tetrahydrofuran-2-yl)-5-hydroxy-4-oxo-4,5-dihydro-3H-pyrazolo[4,3-d][1,2,3]triazine-7-carboxylate

Analogously to General Method C, the intermediate compound (I) 2-(acetoxymethyl)-5-(4-(2-ethoxy-2-oxoacetyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-oxoacetate and 5-(acetoxymethyl)tetrahydrofuran-2,3,4-triyl triacetate. Analogously to General Method J, the intermediate compound (II) 2-(acetoxymethyl)-5-(7-(ethoxycarbonyl)-5-hydroxy-4-oxo-4,5-dihydro-3H-pyrazolo[4,3-d][1,2,3]triazin-3-yl)tetrahydrofuran-3,4-diyl diacetate was prepared from above intermediate compound (I) and hydroxyhydrazine for next step. Analogously to General Method E, the title compound was prepared from the intermediate compound (II) 4.83 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.58 (s, 2H), 2.00 (s, 1H), 1.29 (t, J=8.0 Hz, 3H).

### Example 14. Preparation of 2-(3,4-dihydroxy-5-(hydroxymethyl)tetrahydrofuran-2-yl)-7-hydroxy-5-propyl-2,7-dihydro-1H-pyrazolo[4,3-d][1,2,3]triazin-1-one

Analogously to General Method C, the intermediate compound (I) 2-(acetoxymethyl)-5-(4-butyryl-5,6-dioxo-5,6-dihydro-1,2,3-triazin-1(4H)-yl)tetrahydrofuran-3,4-diyl diacetate was prepared from 6-butyryl-1,2,3-triazine-4,5(3H,6H)-dione and 5-(acetoxymethyl)tetrahydrofuran-2,3,4-triyl triacetate. Analogously to General Method J, the intermediate compound (II) 2-(acetoxymethyl)-5-(5-hydroxy-4-oxo-7-propyl-4,5-dihydro-3H-pyrazolo[4,3-d][1,2,3]triazin-3-yl)tetrahydrofuran-3,4-diyl diacetate was prepared from above intermediate compound (I) and hydroxyhydrazine for next step. Analogously to General Method E, the title compound was prepared from the intermediate compound (II) 4.53 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2H), 2.00 (s, 1H), 1.65 (m, 2H), 0.90 (t, J=8.0 Hz, 3H).

### Example 15. Preparation of 7-amino-3-(3,4-dihydroxy-5-(hydroxymethyl) tetrahydrofuran-2-yl)-5-hydroxy-3H-pyrazolo[4,3-d][1,2,3]triazin-4(5H)-one

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-hydroxy-4-oxo-4,5-dihydro-3H-pyrazolo[4,3-d][1,2,3]triazin-3-yl) tetrahydrofuran-3,4-diyl diacetate was prepared from above intermediate compound (I) and hydroxyhydrazine for next step. Analogously to General Method E, the title compound was prepared from the intermediate compound (I) 4.26 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.00 (s, 1H).

### Example 16. Preparation of N-(3-(3,4-dihydroxy-5-(hydroxymethyl)tetrahydrofuran-2-yl)-5-hydroxy-4-oxo-4,5-dihydro-3H-pyrazolo[4,3-d][1,2,3]triazin-7-yl)-3-(dimethylamino) propanamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-hydroxy-4-oxo-4,5-dihydro-3H-pyrazolo[4,3-d][1,2,3]triazin-3-yl)tetrahy-drofuran-3,4-diyl diacetate was prepared from above intermediate compound (I) and hydroxyhydrazine for next step. Analogously to General Method K, the intermediate compound (III) 2-(acetoxymethyl)-5-(7-(3-(dimethylamino)propanamido)-5-hydroxy-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoyl chloride for next step. Analogously to General Method E, the title compound was prepared from the intermediate compound (III) 5.25 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H), 2.00 (s, 1H).

### Example 17. Preparation of 3-(3,4-dihydroxy-5-(hydroxymethyl)tetrahydrofuran-2-yl)-5-hydroxy-4-oxo-4,5-dihydro-3H-pyrazolo [4,3-d][1,2,3]triazine-7-carbonitrile

To a solution of 2-(acetoxymethyl)-5-(7-chloro-5-hydroxy-4-oxo-4,5-dihydro-3H-pyrazolo[4,3-d][1,2,3]triazin-3-yl)tetrahydrofuran-3,4-diyl diacetate 4.45 g (10 mmol) in acetone (15 mL) was added NaCN 0.70 g (12 mmol) and the mixture was stirred for 5 h. The crude product was purified by flash chromatography to give 2-(acetoxymethyl)-5-(7-cyano-5-hydroxy-4-oxo-4,5-dihydro-3H-pyrazolo[4,3-d][1,2,3]triazin-3-yl)tetrahydrofuran-3,4-diyl diacetate (I) for next step. Analogously to General Method E, the title compound was prepared from the above intermediate compound (I) 4.36 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.00 (s, 1H).

### Example 18. Preparation of 3-(3,4-dihydroxy-5-(hydroxymethyl)tetrahydrofuran-2-yl)-5-hydroxy-7-(piperidin-1-yl)-3H-pyrazolo [4,3-d][1,2,3]triazin-4(5H)-one

To a solution of 2-(acetoxymethyl)-5-(7-chloro-5-hydroxy-4-oxo-4,5-dihydro-3H-pyrazolo[4,3-d][1,2,3]triazin-3-yl)tetrahydrofuran-3,4-diyl diacetate 4.45 g (10 mmol) in THF (15 mL) was added piperidine 2.04 g (24 mmol) and the mixture was stirred for 5 h. The crude product was purified by flash chromatography to give 2-(acetoxymethyl)-5-(5-hydroxy-4-oxo-7-(piperidin-1-yl)-4,5-dihydro-3H-pyrazolo[4,3-d][1,2,3]triazin-3-yl)tetrahydrofuran-3,4-diyl diacetate (I) for next step. Analogously to General Method E, the title compound was prepared from the above intermediate compound (I) 4.94 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 3.11 (s, 4H), 2.00 (s, 1H), 1.65 (d, 6H).

### Example 19. Preparation of 3-(3,4-dihydroxy-5-(hydroxymethyl)tetrahydrofuran-2-yl)-5-hydroxy-7-nitro-3H-pyrazolo[4,3-d][1,2,3]triazin-4(5H)-one

To a mixture of 2-(acetoxymethyl)-5-(5-hydroxy-4-oxo-4,5-dihydro-3H-pyrazolo[4,3-d][1,2,3]triazin-3-yl)tetrahydrofuran-3,4-diyl diacetate 4.11 g (10 mmol) in THF 20 mL were added concentrated HNO₃ 1 mL. The mixture was reacted for overnight. The reaction solution was evaporated and extracted by EtOAc. The crude was separated by silica gel column chromatography to give 2-(acetoxymethyl)-5-(5-hydroxy-7-nitro-4-oxo-4,5-dihydro-3H-pyrazolo[4,3-d][1,2,3]triazin-3-yl) tetrahydrofuran-3,4-diyl diacetate (I) for next step. Analogously to General Method E, the title compound was prepared from the above intermediate compound (I) 4.56 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H, —OH), 3.58 (s, 2H), 2.00 (s, 1H).

### Example 20. Preparation of N-(3-(3,4-dihydroxy-5-(hydroxymethyl)tetrahydrofuran-2-yl)-5-hydroxy-4-oxo-4,5-dihydro-3H-pyrazolo[4,3-d][1,2,3]triazin-7-yl)-4-methoxybenzamide

Analogously to General Method K, 2-(acetoxymethyl)-5-(5-hydroxy-7-(4-methoxybenzamido)-4-oxo-4,5-dihydro-3H-pyrazolo[4,3-d][1,2,3]triazin-3-yl)tetrahydrofuran-3,4-diyl diacetate was prepared from 2-(acetoxy-methyl)-5-(7-amino-5-hydroxy-4-oxo-4,5-dihydro-3H-pyrazolo[4,3-d][1,2,3]triazin-3-yl) tetrahydrofuran-3,4-diyl diacetate and 4-methoxybenzoyl chloride for next step. Analogously to General Method E, the title compound was prepared from the intermediate compound (III) 5.60 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): 69.15 (s, 1H), 7.92 (d, 2H), 7.17 (d, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.83 (s, 3H), 3.66 (m, 2H), 3.65 (s, 1H), 3.58 (s, 2H), 2.00 (s, 1H).

### Example 21. Preparation of 2-(diethoxyphosphoryl)-2-((3-(3,4-dihydroxy-5-(hydroxymethyl)tetrahydrofuran-2-yl)-5-methyl-4-oxo-4,5-dihydro-3H-pyrazolo[4,3-d][1,2,3]triazin-7-yl)amino)acetic acid

To a solution of 2-(acetoxymethyl)-5-(7-chloro-5-methyl-4-oxo-4,5-dihydro-3H-pyrazolo[4,3-d][1,2,3]triazin-3-yl)tetrahydrofuran-3,4-diyl diacetate 4.43 g (10 mmol) in THF (15 ml) was added 2-(diethoxyphosphoryl)-2-(methylamino)acetic acid 5.4 g (24 mmol) and the mixture was stirred for 5 h. The crude product was purified by flash chromatography to give 2-((3-(3,4-diacetoxy-5-(acetoxy-methyl)tetrahydrofuran-2-yl)-5-methyl-4-oxo-4,5-dihydro-3H-pyrazolo[4,3-d][1,2,3]triazin-7-yl) (methyl)amino)-2-(diethoxyphosphoryl)acetic acid (I) for next step. Analogously to General Method E, the title compound was prepared from the above intermediate compound (I) 6.32 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chroma-tography to give the title compound. ¹H NMR (600 MHZ, CDCl₃-d₆): δ 11.00 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 4.07 (m, 4H), 4.00 (s, 1H), 3.95 (s, 3H), 3.66 (m, 2H), 3.65 (s, 1H), 3.58 (s, 2H), 3.50 (d, J=11.5 Hz, 1H), 1.29 (t, 6H).

### Example 22. Preparation of ethyl 3-(3,4-dihydroxy-5-(hydroxymethyl)tetrahydrofuran-2-yl)-5-methyl-4-oxo-4,5-dihydro-3H-pyrazolo[4,3-d][1,2,3]triazine-7-carboxylate

Analogously to General Method C, the intermediate compound (I) 2-(acetoxymethyl)-5-(7-(ethoxycarbonyl)-5-methyl-4-oxo-4,5-dihydro-3H-pyrazolo[4,3-d][1,2,3]triazin-3-yl)tetrahydrofuran-3,4-diyl diacetate was prepared from ethyl 5-methyl-4-oxo-4,5-dihydro-3H-pyrazolo[4,3-d][1,2,3]triazine-7-carboxylate and 5-(acetoxymethyl)tetrahydrofuran-2,3,4-triyl triacetate for next step. Analogously to General Method E, the title compound was prepared from the above intermediate compound (I) 4.81 g (10 mmol) and the solution of methanol/NaOCH₃ 20 mL (1 mol/L). The mixture was reacted for 5 h and added acetic acid to pH˜7. The reaction solution was extracted by EtOAc.

The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.95 (s, 3H), 3.66 (m, 2H), 3.65 (s, 1H), 3.58 (s, 2H), 1.29 (t, J=8.0 Hz, 3H).

### Example 23. Preparation of 3-(3,4-dihydroxy-5-(hydroxymethyl)tetrahydrofuran-2-yl)-5-methyl-7-propyl-3H-pyrazolo [4,3-d][1,2,3]triazin-4(5H)-one

Analogously to General Method C, the intermediate compound (I) 2-(acetoxymethyl)-5-(5-methyl-4-oxo-7-propyl-4,5-dihydro-3H-pyrazolo[4,3-d][1,2,3]triazin-3-yl)tetrahydrofuran-3,4-diyl diacetate was prepared from 5-methyl-7-propyl-3H-pyrazolo[4,3-d][1,2,3]triazin-4(5H)-one and 5-(acetoxymethyl) tetrahydrofuran-2,3,4-triyl triacetate for next step. Analogously to General Method E, the title compound was prepared from the above intermediate compound (I) 4.51 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.66 (m, 2H), 3.65 (s, 1H), 3.58 (s, 2H), 2.44 (t, J=7.1 Hz, 2H), 1.65 (m, 2H), 0.90 (t, J=8.0 Hz, 3H).

### Example 24. Preparation of 7-amino-3-(3,4-dihydroxy-5-(hydroxymethyl) tetrahydrofuran-2-yl)-5-methyl-3H-pyrazolo [4,3-d][1,2,3]triazin-4(5H)-one

Analogously to General Method J, the intermediate compound (I) 2-(acetoxymethyl)-5-(7-amino-5-methyl-4-oxo-4,5-dihydro-3H-pyrazolo[4,3-d][1,2,3]triazin-3-yl)tetrahydrofuran-3,4-diyl diacetate was prepared from 2-(acetoxymethyl)-5-(4-cyano-5,6-dioxo-5,6-dihydro-1,2,3-triazin-1(4H)-yl)tetrahy-drofuran-3,4-diyl diacetate and methylhydrazine for next step. Analogously to General Method E, the title compound was prepared from the intermediate compound (III) 4.23 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.74 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H-), 3.95 (s, 3H), 3.66 (m, 2H), 3.65 (s, 1H), 3.58 (s, 2H).

### Example 25. Preparation of N-(3-(3,4-dihydroxy-5-(hydroxymethyl)tetrahydrofuran-2-yl)-5-methyl-4-oxo-4,5-dihydro-3H-pyrazolo[4,3-d][1,2,3]triazin-7-yl)-3-(dimethylamino) propanamide

Analogously to General Method C, the intermediate compound (I) 2-(acetoxymethyl)-5-(4-butyryl-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-methyl-4-oxo-4,5-dihydro-3H-pyrazolo[4,3-d][1,2,3]triazin-3-yl)tetrahy-drofuran-3,4-diyl diacetate was prepared from the intermediate compound (I) and methylhydrazine for next step. Analogously to General Method K, the intermediate compound (III) 2-(acetoxymethyl)-5-(7-(3-(dimethylamino)propanamido)-5-methyl-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoylchloride for next step. Analogously to General Method E, the title compound was prepared from the intermediate compound (III) 5.23 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H).

### Example 26. Preparation of 3-(3,4-dihydroxy-5-(hydroxymethyl)tetrahydrofuran-2-yl)-5-methyl-4-oxo-4,5-dihydro-3H-pyrazolo[4,3-d][1,2,3]triazine-7-carbonitrile

Analogously to General Method C, the intermediate compound (I) 2-(acetoxymethyl)-5-(7-cyano-5-methyl-4-oxo-4,5-dihydro-3H-pyrazolo[4,3-d][1,2,3]triazin-3-yl)tetrahydrofuran-3,4-diyl diacetate was prepared from 5-methyl-4-oxo-4,5-dihydro-3H-pyrazolo[4,3-d][1,2,3]triazine-7-carbonitrile and 5-(acetoxymethyl)tetrahydrofuran-2,3,4-triyl triacetate for next step. Analogously to General Method E, the title compound was prepared from the intermediate compound (I) 4.34 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.66 (m, 2H), 3.65 (s, 1H), 3.58 (s, 2H).

### Example 27. Preparation of 3-(3,4-dihydroxy-5-(hydroxymethyl)tetrahydrofuran-2-yl)-5-methyl-7-(piperidin-1-yl)-3H-pyrazolo[4,3-d][1,2,3]triazin-4(5H)-one

Analogously to General Method C, the intermediate compound (I) 2-(acetoxymethyl)-5-(5-methyl-4-oxo-7-(piperidin-1-yl)-4,5-dihydro-3H-pyrazolo[4,3-d][1,2,3]triazin-3-yl)tetrahydrofuran-3,4-diyl diacetate was prepared from 5-methyl-7-(piperidin-1-yl)-3H-pyrazolo[4,3-d][1,2,3]triazin-4(5H)-one and 5-(acetoxy-methyl)tetrahydrofuran-2,3,4-triyl triacetate for next step. Analogously to General Method E, the title compound was prepared from the intermediate compound (I) 4.92 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.66 (m, 2H), 3.65 (s, 1H), 3.58 (s, 2H), 3.11 (s, 4H), 1.65 (d, 6H).

### Example 28. Preparation of 3-(3,4-dihydroxy-5-(hydroxymethyl)tetrahydrofuran-2-yl)-5-methyl-7-nitro-3H-pyrazolo [4,3-d][1,2,3]triazin-4(5H)-one

To a mixture of 5-methyl-7-nitro-3H-pyrazolo[4,3-d][1,2,3]triazin-4(5H)-one 1.96 g (10 mmol) in THF 20 mL were added concentrated HNO₃ 1 mL. The mixture was reacted for overnight. The reaction solution was evaporated and extracted by EtOAc. The crude was separated by silica gel column chromatography to give 2-(acetoxymethyl)-5-(5-methyl-7-nitro-4-oxo-4,5-dihydro-3H-pyrazolo[4,3-d][1,2,3]triazin-3-yl)tetrahydrofuran-3,4-diyl diacetate (I) for next step. Analogously to General Method C, the intermediate compound (II) 2-(acetoxymethyl)-5-(5-methyl-7-nitro-4-oxo-4,5-dihydro-3H-pyrazolo[4,3-d][1,2,3]triazin-3-yl)tetrahydrofuran-3,4-diyl diacetate was prepared from above compound (I) and 5-(acetoxy-methyl)tetrahydrofuran-2,3,4-triyl triacetate for next step. Analogously to General Method E, the title compound was prepared from the intermediate compound (II) 4.54 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.66 (m, 2H), 3.65 (s, 1H), 3.58 (s, 2H).

### Example 29. Preparation of N-(3-(3,4-dihydroxy-5-(hydroxymethyl)tetrahydrofuran-2-yl)-5-methyl-4-oxo-4,5-dihydro-3H-pyrazolo[4,3-d][1,2,3]triazin-7-yl)-4-methoxybenzamide

Analogously to General Method C, the intermediate compound (I) 2-(acetoxymethyl)-5-(7-(4-methoxy-benzamido)-5-methyl-4-oxo-4,5-dihydro-3H-pyrazolo[4,3-d][1,2,3]triazin-3-yl)tetrahydrofuran-3,4-diyl diacetate was prepared from 4-methoxy-N-(5-methyl-4-oxo-4,5-dihydro-3H-pyrazolo[4,3-d][1, 2,3]triazin-7-yl)benzamide and 5-(acetoxymethyl)tetrahydrofuran-2,3,4-triyl triacetate for next step. Analogously to General Method E, the title compound was prepared from the intermediate compound (I) 5.58 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 7.92 (d, 2H), 7.17 (d, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.95 (s, 3H), 3.83 (s, 3H), 3.66 (m, 2H), 3.65 (s, 1H), 3.58 (s, 2H).

### Example 30. Preparation of ethyl 3-(3,4-dihydroxy-5-(hydroxymethyl) tetrahydrofuran-2-yl)-5-(hydroxymethyl)-4-oxo-4,5-dihydro-3H-pyrazolo [4,3-d][1,2,3]triazine-7-carboxylate

Analogously to General Method C, the intermediate compound (I) 2-(acetoxymethyl)-5-(4-(2-ethoxy-2-oxo-acetyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-oxoacetate and 5-(acetoxymethyl)tetrahydro-furan-2,3,4-triyl triacetate for next step. Analogously to General Method J, the intermediate compound (II) 2-(acetoxymethyl)-5-(7-(ethoxycarbonyl)-5-(hydroxymethyl)-4-oxo-4,5-dihydro-3H-pyrazolo[4,3-d][1,2,3]triazin-3-yl)tetrahydrofuran-3,4-diyl diacetate was prepared from above intermediate compound (I) and hydrazinylmethanol for next step. Analogously to General Method E, the title compound was prepared from the intermediate compound (II) 4.97 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.60 (s, 1H), 3.58 (s, 2H), 1.29 (t, J=8.0 Hz, 3H).

### Example 31. Preparation of 3-(3,4-dihydroxy-5-(hydroxymethyl)tetrahydrofuran-2-yl)-5-(hydroxymethyl)-7-propyl-3H-pyrazolo[4,3-d][1,2,3]triazin-4(5H)-one

Analogously to General Method C, the intermediate compound (I) 2-(acetoxymethyl)-5-(4-butyryl-5,6-dioxo-5,6-dihydro-1,2,3-triazin-1(4H)-yl)tetrahydrofuran-3,4-diyl diacetate was prepared from 6-butyryl-1,2,3-triazine-4,5(3H,6H)-dione and 5-(acetoxymethyl)tetrahydrofuran-2,3,4-triyl triacetate for next step. Analogously to General Method J, the intermediate compound (II) 2-(acetoxymethyl)-5-(5-(hydroxymethyl)-4-oxo-7-propyl-4,5-dihydro-3H-pyrazolo[4,3-d][1,2,3]triazin-3-yl)tetrahydrofuran-3,4-diyl diacetate was prepared from above intermediate compound (I) and hydrazinylmethanol for next step. Analogously to General Method E, the title compound was prepared from the intermediate compound (II) 4.67 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1 H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.60 (s, 1H), 3.58 (s, 2H), 2.44 (t, J=7.1 Hz, 2H), 1.65 (m, 2H), 0.90 (t, J=8.0 Hz, 3H).

### Example 32. Preparation of 7-amino-3-(3,4-dihydroxy-5-(hydroxymethyl) tetrahydrofuran-2-yl)-5-(hydroxymethyl)-3H-pyrazolo [4,3-d][1,2,3]triazin-4(5H)-one

Analogously to General Method C, the intermediate compound (I) 2-(acetoxymethyl)-5-(4-carbamoyl-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carboxamide and 5-(acetoxymethyl)tetrahydrofuran-2,3,4-triyl triacetate for next step. Analogously to General Method J, the intermediate compound (II) 2-(acetoxymethyl)-5-(7-amino-5-(hydroxymethyl)-4-oxo-4,5-dihydro-3H-pyrazolo[4,3-d][1,2,3]triazin-3-yl)tetrahydrofuran-3,4-diyl diacetate was prepared from above intermediate compound (I) and hydrazinylmethanol for next step. Analogously to General Method E, the title compound was prepared from the intermediate compound (II) 4.40 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.60 (s, 1H), 3.58 (s, 2H).

### Example 33. Preparation of N-(3-(3,4-dihydroxy-5-(hydroxymethyl)tetrahydrofuran-2-yl)-5-(hydroxymethyl)-4-oxo-4,5-dihydro-3H-pyrazolo[4,3-d][1,2,3]triazin-7-yl)-3-(dimethylamino) propanamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-(hydroxymethyl)-4-oxo-4,5-dihydro-3H-pyrazolo[4,3-d][1,2,3]triazin-3-yl) tetrahydrofuran-3,4-diyl diacetate was prepared from above intermediate compound (I) and hydrazinylmethanol for next step. Analogously to General Method K, the intermediate compound (III) 2-(acetoxymethyl)-5-(7-(3-(dimethylamino)propanamido)-5-(hydroxymethyl)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoyl chloride for next step. Analogously to General Method E, the title compound was prepared from the intermediate compound (II) 5.39 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.64 (s, 1H), 3.60 (s, 1H), 3.58 (s, 2H), 2.84 (s, 6H), 2.50 (t, J=7.1 Hz, 2H).

### Example 34. Preparation of 3-(3,4-dihydroxy-5-(hydroxymethyl)tetrahydrofuran-2-yl)-5-(hydroxymethyl)-4-oxo-4,5-dihydro-3H-pyrazolo [4,3-d][1,2,3]triazine-7-carbonitrile

Analogously to General Method C, the intermediate compound (I) 2-(acetoxymethyl)-5-(4-(cyanocarbonyl)-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonyl cyanide and 5-(acetoxymethyl) tetrahydrofuran-2,3,4-triyl triacetate for next step. Analogously to General Method J, the intermediate compound (II) 2-(acetoxymethyl)-5-(7-cyano-5-(hydroxymethyl)-4-oxo-4,5-dihydro-3H-pyrazolo[4,3-d][1,2,3]triazin-3-yl)tetrahydrofuran-3,4-diyl diacetate was prepared from above intermediate compound (I) and hydrazinylmethanol for next step. Analogously to General Method E, the title compound was prepared from the intermediate compound (II) 4.50 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.60 (s, 1H), 3.58 (s, 2H).

### Example 35. Preparation of 3-(3,4-dihydroxy-5-(hydroxymethyl)tetrahydrofuran-2-yl)-5-(hydroxymethyl)-7-(piperidin-1-yl)-3H-pyrazolo[4,3-d][1,2,3]triazin-4(5H)-one

Analogously to General Method C, the intermediate compound (I) 2-(acetoxymethyl)-5-(5,6-dioxo-4-(piperidine-1-carbonyl)-5,6-dihydro-1,2,3-triazin-1(4H)-yl)tetrahydrofuran-3,4-diyl diacetate was prepared from 6-(piperidine-1-carbonyl)-1,2,3-triazine-4,5(3H,6H)-dione and 5-(acetoxymethyl) tetrahydrofuran-2,3,4-triyl triacetate for next step. Analogously to General Method J, the intermediate compound (II) 2-(acetoxymethyl)-5-(5-(hydroxymethyl)-4-oxo-7-(piperidin-1-yl)-4,5-dihydro-3H-pyrazolo[4,3-d][1,2,3]triazin-3-yl)tetrahydro-furan-3,4-diyl diacetate was prepared from above intermediate compound (I) and hydrazinylmethanol for next step. Analogously to General Method E, the title compound was prepared from the intermediate compound (II) 5.08 g (10 m mol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.60 (s, 1H), 3.58 (s, 2H), 3.11 (s, 4H), 1.65 (d, 6H).

### Example 36. Preparation of 3-(3,4-dihydroxy-5-(hydroxymethyl)tetrahydrofuran-2-yl)-5-(hydroxymethyl)-7-nitro-3H-pyrazolo [4,3-d][1,2,3]triazin-4(5H)-one

Analogously to General Method C, the intermediate compound (I) 2-(acetoxymethyl)-5-(4-(nitrocarbonyl)-5,6-dioxo-5,6-dihydro-1,2,3-triazin-1(4H)-yl)tetrahydrofuran-3,4-diyl diacetate was prepared from 6-(piperidine-1-carbonyl)-1,2,3-triazine-4,5(3H,6H)-dione and 5-(acetoxymethyl) tetrahydrofuran-2,3,4-triyl triacetate for next step. Analogously to General Method J, the intermediate compound (II) 2-(acetoxymethyl)-5-(5-(hydroxymethyl)-7-nitro-4-oxo-4,5-dihydro-3H-pyrazolo[4,3-d][1,2,3]triazin-3-yl)tetrahydrofuran-3,4-diyl diacetate was prepared from above intermediate compound (I) and hydrazinylmethanol for next step. Analogously to General Method E, the title compound was prepared from the intermediate compound (II) 4.70 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.60 (s, 1H), 3.58 (s, 2H).

### Example 37. Preparation of N-(3-(3,4-dihydroxy-5-(hydroxymethyl)tetrahydrofuran-2-yl)-5-(hydroxymethyl)-4-oxo-4,5-dihydro-3H-pyrazolo[4,3-d][1,2,3]triazin-7-yl)-4-methoxybenzamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-(hydroxymethyl)-4-oxo-4,5-dihydro-3H-pyrazolo[4,3-d][1,2,3]triazin-3-yl) tetrahydrofuran-3,4-diyl diacetate was prepared from above intermediate compound (I) and hydrazinylmethanol for next step. Analogously to General Method K, the intermediate compound (III) 2-(acetoxymethyl)-5-(5-(hydroxymethyl)-7-(4-methoxybenzamido)-4-oxo-4,5-dihydro-3H-pyrazolo [4,3-d][1,2,3]triazin-3-yl)tetrahydrofuran-3,4-diyl diacetate was prepared from above intermediate compound (II) and 4-methoxybenzoyl chloride for next step. Analogously to General Method E, the title compound was prepared from the intermediate compound (III) 5.74 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ. 9.15 (s, 1H), 7.92 (d, 2H), 7.17 (d, 2H), 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.83 (s, 3H), 3.66 (m, 2H), 3.65 (s, 1H), 3.60 (s, 1H), 3.58 (s, 2H).

### Example 38. Preparation of 2-((5-cyclopropyl-3-(3,4-dihydroxy-5-(hydroxymethyl) tetrahydro-furan-2-yl)-4-oxo-4,5-dihydro-3H-pyrazolo[4,3-d][1,2,3]triazin-7-yl)amino)-2-(diethoxyphosphoryl)acetic acid

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-cyclopropyl-4-oxo-4,5-dihydro-3H-pyrazolo[4,3-d][1,2,3]triazin-3-yl) tetrahydrofuran-3,4-diyl diacetate was prepared from above intermediate compound (I) and cyclopropylhydrazine for next step. Analogously to General Method K, the intermediate compound (III) 2-((5-cyclopropyl-3-(3,4-diacetoxy-5-(acetoxymethyl)tetrahydrofuran-2-yl)-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 6.44 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 11.00 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 4.07 (m, 4H), 4.00 (s, 1H), 3.66 (m, 2H), 3.65 (s, 1H), 3.58 (s, 2H), 3.50 (d, J=11.5 Hz, 1H), 2.42 (m, J=7.0 Hz, 1H), 1.29 (t, 6H), 0.66 (m, 4H).

### Example 39. Preparation of ethyl 5-cyclopropyl-3-(3,4-dihydroxy-5-(hydroxymethyl) tetrahydrofuran-2-yl)-4-oxo-4,5-dihydro-3H-pyrazolo[4,3-d][1,2,3]triazine-7-carboxylate

Analogously to General Method C, the intermediate compound (I) 2-(acetoxymethyl)-5-(5-cyclopropyl-7-(ethoxycarbonyl)-4-oxo-4,5-dihydro-3H-pyrazolo[4,3-d][1,2,3]triazin-3-yl)tetrahydrofuran-3,4-diyl diacetate was prepared from ethyl 5-cyclopropyl-4-oxo-4,5-dihydro-3H-pyrazolo[4,3-d][1,2,3]triazine-7-carboxylate and 5-(acetoxymethyl)tetrahydrofuran-2,3,4-triyl triacetate for next step. Analogously to General Method E, the title compound was prepared from the intermediate compound (I) 5.07 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.58 (s, 2H), 2.42 (m, J=7.0 Hz, 1H), 1.29 (t, J=8.0 Hz, 3H), 0.66 (m, 4H).

### Example 40. Preparation of 5-cyclopropyl-3-(3,4-dihydroxy-5-(hydroxymethyl) tetrahydrofuran-2-yl)-7-propyl-3H-pyrazolo[4,3-d][1,2,3]triazin-4(5H)-one

Analogously to General Method C, the intermediate compound (I) 2-(acetoxymethyl)-5-(5-cyclopropyl-4-oxo-7-propyl-4,5-dihydro-3H-pyrazolo[4,3-d][1,2,3]triazin-3-yl)tetrahydrofuran-3,4-diyl diacetate was prepared from 5-cyclopropyl-7-propyl-3H-pyrazolo[4,3-d][1,2,3]triazin-4(5H)-one and 5-(acetoxymethyl)tetrahy-drofuran-2,3,4-triyl triacetate for next step. Analogously to General Method E, the title compound was prepared from the intermediate compound (I) 4.77 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2 H), 2.42 (m, J=7.0 Hz, 1H), 1.65 (m, 2H), 0.90 (t, J=8.0 Hz, 3H), 0.66 (m, 4H).

### Example 41. Preparation of 7-amino-5-cyclopropyl-3-(3,4-dihydroxy-5-(hydroxymethyl)tetrahydrofuran-2-yl)-3H-pyrazolo[4,3-d][1,2,3]triazin-4(5H)-one

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl) tetrahydrofuran-2,3,4-triyl triacetate for next step. Analogously to General Method J, the intermediate compound (II) 2-(acetoxymethyl)-5-(7-amino-5-cyclopropyl-4-oxo-4,5-dihydro-3H-pyrazolo[4,3-d][1,2,3]triazin-3-yl) tetrahydrofuran-3,4-diyl diacetate was prepared from above intermediate compound (I) and cyclopropylhydrazine for next step. Analogously to General Method E, the title compound was prepared from the intermediate compound (III) 5.50 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.42 (m, J=7.0 Hz, 1H), 0.66 (m, 4H).

### Example 42. Preparation of N-(5-cyclopropyl-3-(3,4-dihydroxy-5-(hydroxymethyl) tetrahydrofuran-2-yl)-4-oxo-4,5-dihydro-3H-pyrazolo[4,3-d][1,2,3]triazin-7-yl)-3-(dimethylamino) propanamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(4-(N-(3-(dimethylamino)propanoyl)carbamimidoyl)-5,6-dioxo-5,6-dihydro-1,2,3-triazin-1(4H)-yl)tetrahydrofuran-3,4-diyl diacetate was prepared from above intermediate compound (I) and cyclopropylhydrazine for next step. Analogously to General Method K, the intermediate compound (III) 2-(acetoxymethyl)-5-(5-cyclopropyl-7-(3-(dimethylamino)propanamido)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoyl chloride for next step. Analogously to General Method E, the title compound was prepared from the intermediate compound (III) 5.49 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 9.15 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H), 2.42 (m, J=7.0 Hz, 1H), 0.66 (m, 4H).

### Example 43. Preparation of 5-cyclopropyl-3-(3,4-dihydroxy-5-(hydroxymethyl) tetrahydrofuran-2-yl)-4-oxo-4,5-dihydro-3H-pyrazolo[4,3-d][1,2,3]triazine-7-carbonitrile

Analogously to General Method C, the intermediate compound (I) 2-(acetoxymethyl)-5-(7-cyano-5-cyclopropyl-4-oxo-4,5-dihydro-3H-pyrazolo[4,3-d][1,2,3]triazin-3-yl)tetrahydrofuran-3,4-diyl diacetate was prepared from 5-cyclopropyl-4-oxo-4,5-dihydro-3H-pyrazolo[4,3-d][1,2,3]triazine-7-carbonitrile and 5-(acetoxy-methyl)tetrahydrofuran-2,3,4-triyl triacetate for next step. Analogously to General Method E, the title compound was prepared from the intermediate compound (I) 4.60 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.42 (m, J=7.0 Hz, 1H), 0.66 (m, 4H).

### Example 44. Preparation of 5-cyclopropyl-3-(3,4-dihydroxy-5-(hydroxymethyl) tetrahydrofuran-2-yl)-7-(piperidin-1-yl)-3H-pyrazolo[4,3-d][1,2,3]triazin-4(5H)-one

Analogously to General Method C, the intermediate compound (I) 2-(acetoxymethyl)-5-(5-cyclopropyl-4-oxo-7-(piperidin-1-yl)-4,5-dihydro-3H-pyrazolo[4,3-d][1,2,3]triazin-3-yl)tetrahydrofuran-3,4-diyl diacetate was prepared from 5-cyclopropyl-3-(3,4-dihydroxy-5-(hydroxymethyl)tetrahydrofuran-2-yl)-7-(piperidin-1-yl)-3H-pyrazolo[4,3-d][1,2,3]triazin-4(5H)-one and 5-(acetoxymethyl) tetrahydrofuran-2,3,4-triyl triacetate for next step. Analogously to General Method E, the title compound was prepared from the intermediate compound (I) 5.18 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 3.11 (s, 4H), 2.42 (m, J=7.0 Hz, 1H), 1.65 (d, 6H), 0.66 (m, 4H).

### Example 45. Preparation of 5-cyclopropyl-3-(3,4-dihydroxy-5-(hydroxymethyl) tetrahydrofuran-2-yl)-7-nitro-3H-pyrazolo[4,3-d][1,2,3]triazin-4(5H)-one

Analogously to General Method C, the intermediate compound (I) 2-(acetoxymethyl)-5-(5-cyclopropyl-7-nitro-4-oxo-4,5-dihydro-3H-pyrazolo[4,3-d][1,2,3]triazin-3-yl)tetrahydrofuran-3,4-diyl diacetate was prepared from 5-cyclopropyl-7-nitro-3H-pyrazolo[4,3-d][1,2,3]triazin-4(5H)-one and 5-(acetoxymethyl)tetrahydro-furan-2,3,4-triyl triacetate for next step. Analogously to General Method E, the title compound was prepared from the intermediate compound (I) 4.80 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.42 (m, J=7.0 Hz, 1H), 0.66 (m, 4H).

### Example 46. Preparation of N-(5-cyclopropyl-3-(3,4-dihydroxy-5-(hydroxymethyl) tetrahydrofuran-2-yl)-4-oxo-4,5-dihydro-3H-pyrazolo[4,3-d][1,2,3]triazin-7-yl)-4-methoxybenzamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl) tetrahydrofuran-2,3,4-triyl triacetate for next step. Analogously to General Method J, the intermediate compound (II) 2-(acetoxymethyl)-5-(7-amino-5-cyclopropyl-4-oxo-4,5-dihydro-3H-pyrazolo[4,3-d][1,2,3]triazin-3-yl) tetrahydrofuran-3,4-diyl diacetate was prepared from above intermediate compound (I) and cyclopropylhydrazine for next step. Analogously to General Method K, the intermediate compound (III) 2-(acetoxymethyl)-5-(5-cyclopropyl-7-(4-methoxybenzamido)-4-oxo-4,5-dihydro-3H-pyrazolo[4,3-d][1,2,3]triazin-3-yl)tetrahydrofuran-3,4-diyl diacetate was prepared from above intermediate compound (II) and 4-methoxybenzoyl chloride for next step. Analogously to General Method E, the title compound was prepared from the intermediate compound (III) 5.84 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 7.92 (d, 2H), 7.17 (d, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.83 (s, 3H), 3.66 (m, 2H), 3.65 (s, 1H), 3.58 (s, 2H), 2.42 (m, J=7.0 Hz, 1H), 0.66 (m, 4H).

### Example 47. Preparation of 2-((5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazin-7-yl) (methyl)amino)-2-(diethoxyphosphoryl)acetic acid

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-(2-amino-2-oxoethyl)-4-oxo-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method K, the intermediate compound (III) 2-((5-(2-amino-2-oxoethyl)-3-(3,4-diacetoxy-5-(acetoxymethyl)tetrahydrofuran-2-yl)-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 6.61 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 11.00 (s, 1H), 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 4.07 (m, 4H), 3.66 (m, 2H), 3.65 (s, 1H), 3.58 (s, 2H), 3.50 (d, J=11.5 Hz, 1H), 2.68 (s, 3H), 1.29 (t, 6H).

### Example 48. Preparation of ethyl 5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate

Analogously to General Method C, the intermediate compound (I) 2-(acetoxymethyl)-5-(4-(2-ethoxy-2-oxo-acetyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-oxoacetate and 5-(acetoxymethyl)tetrahydro-furan-2,3,4-triyl triacetate for next step. Analogously to General Method J, the intermediate compound (II) 2-(acetoxymethyl)-5-(5-(2-amino-2-oxoethyl)-7-(ethoxycarbonyl)-4-oxo-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method E, the title compound was prepared from the intermediate compound (II) 4.70 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.58 (s, 2H), 1.29 (t, J=8.0 Hz, 3H).

### Example 49. Preparation of 2-(3-(3,4-dihydroxy-5-(hydroxymethyl)tetrahydrofuran-2-yl)-4-oxo-7-propyl-3H-pyrazolo [4,3-d][1,2,3]triazin-5(4H)-yl)acetamide

Analogously to General Method C, the intermediate compound (I) 2-(acetoxymethyl)-5-(4-butyryl-5,6-dioxo-5,6-dihydro-1,2,3-triazin-1(4H)-yl)tetrahydrofuran-3,4-diyl diacetate was prepared from 6-butyryl-1,2,3-triazine-4,5(3H,6H)-dione and 5-(acetoxymethyl)tetrahydrofuran-2,3,4-triyl triacetate for next step. Analogously to General Method J, the intermediate compound (II) 2-(acetoxymethyl)-5-(5-(2-amino-2-oxoethyl)-4-oxo-7-propyl-4,5-dihydro-3H-pyrazolo[4,3-d][1,2,3]triazin-3-yl) tetrahydrofuran-3,4-diyl diacetateate was prepared from above intermediate compound (I) and 2-hydrazinylacetamide for next step. Analogously to General Method E, the title compound was prepared from the intermediate compound (II) 4.70 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2H), 1.65 (m, 2H), 0.90 (t, J=8.0 Hz, 3H).

### Example 50. Preparation of 2-(7-amino-3-(3,4-dihydroxy-5-(hydroxymethyl) tetrahydrofuran-2-yl)-4-oxo-3H-pyrazolo[4,3-d][1,2,3]triazin-5(4H)-yl)acetamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-(2-amino-2-oxoethyl)-4-oxo-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method E, the title compound was prepared from the intermediate compound (II) 4.67 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 51. Preparation of N-(5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazin-7-yl)-3-(dimethylamino)propanamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-(2-amino-2-oxoethyl)-4-oxo-4,5-dihydro-3H-pyrazolo[4,3-d][1,2,3]triazin-3-yl)tetrahydrofuran-3,4-diyl diacetate was prepared from the intermediate compound (I) and 2-hydrazinylacetamide for next step. Analogously to General Method K, the intermediate compound (III) 2-(acetoxymethyl)-5-(5-(2-amino-2-oxoethyl)-7-(3-(dimethylamino)propanamido)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoylchloride for next step. Analogously to General Method E, the title compound was prepared from the intermediate compound (III) 4.77 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 9.15 (s, 1H), 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H).

### Example 52. Preparation of 2-(7-cyano-3-(3,4-dihydroxy-5-(hydroxymethyl) tetrahydrofuran-2-yl)-4-oxo-3H-pyrazolo[4,3-d][1,2,3]triazin-5(4H)-yl)acetamide

Analogously to General Method C, the intermediate compound (I) 2-(acetoxymethyl)-5-(4-(cyanocarbonyl)-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonyl cyanide and 5-(acetoxymethyl) tetrahydrofuran-2,3,4-triyl triacetate for next step. Analogously to General Method J, the intermediate compound (II) 2-(acetoxymethyl)-5-(5-(2-amino-2-oxoethyl)-7-cyano-4-oxo-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method E, the title compound was prepared from the intermediate compound (II) 4.77 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 53. Preparation of 2-(3-(3,4-dihydroxy-5-(hydroxymethyl)tetrahydrofuran-2-yl)-4-oxo-7-(piperidin-1-yl)-3H-pyrazolo[4,3-d][1,2,3]triazin-5(4H)-yl)acetamide

Analogously to General Method C, the intermediate compound (I) 2-(acetoxymethyl)-5-(4-(imino (piperidin-1-yl)methyl)-5,6-dioxo-5,6-dihydro-1,2,3-triazin-1(4H)-yl)tetrahydrofuran-3,4-diyl diacetate was prepared from 6-(imino(piperidin-1-yl)methyl)-1,2,3-triazine-4,5(3H,6H)-dione and 5-(acetoxymethyl)tetrahy-drofuran-2,3,4-triyl triacetate for next step. Analogously to General Method J, the intermediate compound (II) 2-(acetoxymethyl)-5-(5-(2-amino-2-oxoethyl)-4-oxo-7-(piperidin-1-yl)-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method E, the title compound was prepared from the intermediate compound (II) 5.35 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 3.11 (s, 4H), 1.65 (d, 6H).

### Example 54. Preparation of 2-(3-(3,4-dihydroxy-5-(hydroxymethyl)tetrahydrofuran-2-yl)-7-nitro-4-oxo-3H-pyrazolo [4,3-d][1,2,3]triazin-5(4H)-yl)acetamide

Analogously to General Method C, the intermediate compound (I) 2-(acetoxymethyl)-5-(5-(2-amino-2-oxoethyl)-7-nitro-4-oxo-4,5-dihydro-3H-pyrazolo[4,3-d][1,2,3]triazin-3-yl)tetrahydrofuran-3,4-diyl diacetate was prepared from 2-(7-nitro-4-oxo-3H-pyrazolo[4,3-d][1,2,3]triazin-5(4H)-yl) acetamide and 5-(acetoxy-methyl)tetrahydrofuran-2,3,4-triyl triacetate for next step. Analogously to General Method J, the intermediate compound (II) 2-(acetoxymethyl)-5-(5-(2-amino-2-oxoethyl)-7-nitro-4-oxo-4,5-dihydro-3H-pyrazolo[4,3-d][1,2,3]triazin-3-yl)tetrahydrofuran-3,4-diyl diacetate was prepared from above intermediate compound (I) and 2-hydrazinylacetamide for next step. Analogously to General Method E, the title compound was prepared from the intermediate compound (II) 4.97 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.16 (s, 2H), 5.93 (d, J=7.0 Hz, 1H), 5.60 (s, 2H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 55. Preparation of 3-(3,4-dihydroxy-5-(hydroxymethyl)tetrahydrofuran-2-yl)-4-oxo-7-propyl-3H-pyrazolo [4,3-d][1,2,3]triazine-5(4H)-carboxamide

Analogously to General Method C, the intermediate compound (I) 2-(acetoxymethyl)-5-(4-butyryl-5,6-dioxo-5,6-dihydro-1,2,3-triazin-1(4H)-yl)tetrahydrofuran-3,4-diyl diacetate was prepared from 6-butyryl-1,2,3-triazine-4,5(3H,6H)-dione and 5-(acetoxymethyl)tetrahydrofuran-2,3,4-triyl triacetate for next step. Analogously to General Method J, the intermediate compound (II) 2-(acetoxymethyl)-5-(5-carbamoyl-4-oxo-7-propyl-4,5-dihydro-3H-pyrazolo[4,3-d][1,2,3]triazin-3-yl)tetrahydrofuran-3,4-diyldiacetate was prepared from above intermediate compound (I) and hydrazinecarboxamide for next step. Analogously to General Method E, the title compound was prepared from the intermediate compound (II) 4.80 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2H), 1.65 (m, 2H), 0.90 (t, J=8.0 Hz, 3H).

### Example 56. Preparation of 7-amino-3-(3,4-dihydroxy-5-(hydroxymethyl) tetrahydrofuran-2-yl)-4-oxo-3H-pyrazolo[4,3-d][1,2,3]triazine-5(4H)-carboxamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamoyl-4-oxo-4,5-dihydro-3H-pyrazolo[4,3-d][1,2,3]triazin-3-yl) tetrahydrofuran-3,4-diyl diacetate was prepared from above intermediate compound (I) and hydrazinecarboxamide for next step. Analogously to General Method E, the title compound was prepared from the intermediate compound (II) 4.53 g (10 mmol) and the solution of methanol/NaOCH₃ 20 mL (1 mol/L). The mixture was reacted for 5 h and added acetic acid to pH˜7.

The reaction solution was extracted by EtOAc. The crude was separated by silica gel column chromatography to give the title compound. ¹H NMR (600 MHZ, CDCl₃-d₆): δ 7.74 (s, 2H), 7.68 (s, 2H), 5.93 (d, J=7.0 Hz, 1H), 4.51 (t, J=7.0 Hz, 1H), 4.40 (m, J=7.0 Hz, 1 H), 4.28 (t, J=7.0 Hz, 1H), 3.66 (m, 2H), 3.65 (s, 1H), 3.58 (s, 2H).

### Example 57. Preparation of 3-(3,4-dihydroxy-5-(hydroxymethyl)tetrahydrofuran-2-yl)-7-(3-(dimethylamino)propanamido)-4-oxo-3H-pyrazolo [4,3-d][1,2,3]triazine-5(4H)-carboxamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamoyl-4-oxo-4,5-dihydro-3H-pyrazolo[4,3-d][1,2,3]triazin-3-yl) tetrahydrofuran-3,4-diyl diacetate was prepared from the intermediate compound (I) and hydrazinecarboxamide for next step. Analogously to General Method K, the intermediate compound (III) 2-(acetoxymethyl)-5-(5-carbamoyl-7-(3-(dimethylamino)propanamido)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoylchloride for next step. Analogously to General Method E, the title compound was prepared from the intermediate compound (III) 5.52 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H).

### Example 58. Preparation of 7-cyano-3-(3,4-dihydroxy-5-(hydroxymethyl) tetrahydrofuran-2-yl)-4-oxo-3H-pyrazolo[4,3-d][1,2,3]triazine-5(4H)-carboxamide

Analogously to General Method C, the intermediate compound (I) 2-(acetoxymethyl)-5-(4-(cyano (imino)methyl)-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbimidoyl cyanide and 5-(acetoxymethyl) tetrahydrofuran-2,3,4-triyl triacetate for next step. Analogously to General Method J, the intermediate compound (II) 2-(acetoxy-methyl)-5-(5-carbamoyl-7-cyano-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarboxamide for next step. Analogously to General Method E, the title compound was prepared from the intermediate compound (II) 4.63 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 59. Preparation of 3-(3,4-dihydroxy-5-(hydroxymethyl)tetrahydrofuran-2-yl)-4-oxo-7-(piperidin-1-yl)-3H-pyrazolo[4,3-d][1,2,3]triazine-5(4H)-carboxamide

Analogously to General Method C, the intermediate compound (I) 2-(acetoxymethyl)-5-(4-(imino (piperidin-1-yl)methyl)-5,6-dioxo-5,6-dihydro-1,2,3-triazin-1(4H)-yl)tetrahydrofuran-3,4-diyl diacetate was prepared from 6-(imino(piperidin-1-yl)methyl)-1,2,3-triazine-4,5(3H,6H)-dione and 5-(acetoxymethyl)tetrahy-drofuran-2,3,4-triyl triacetate for next step. Analogously to General Method J, the intermediate compound (II) 2-(acetoxymethyl)-5-(5-carbamoyl-4-oxo-7-(piperidin-1-yl)-4,5-dihydro-3H-pyrazolo[4,3-d][1,2,3]triazin-3-yl)tetrahydrofuran-3,4-diyl diacetate was prepared from above intermediate compound (I) and hydrazinecarboxamide for next step. Analogously to General Method E, the title compound was prepared from the intermediate compound (II) 5.21 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 3.11 (s, 4H), 1.65 (d, 6H).

### Example 60. Preparation of 3-(3,4-dihydroxy-5-(hydroxymethyl)tetrahydrofuran-2-yl)-7-nitro-4-oxo-3H-pyrazolo[4,3-d][1,2,3]triazine-5(4H)-carboxamide

Analogously to General Method C, the intermediate compound (I) 2-(acetoxymethyl)-5-(4-(imino (nitro)methyl)-5,6-dioxo-5,6-dihydro-1,2,3-triazin-1(4H)-yl)tetrahydrofuran-3,4-diyl diacetate was prepared from 6-(imino(nitro)methyl)-1,2,3-triazine-4,5(3H,6H)-dione and 5-(acetoxymethyl) tetrahydrofuran-2,3,4-triyl triacetate for next step. Analogously to General Method J, the intermediate compound (II) 2-(acetoxymethyl)-5-(5-carbamoyl-7-nitro-4-oxo-4,5-dihydro-3H-pyrazolo [4,3-d][1,2,3]triazin-3-yl)tetrahydrofuran-3,4-diyl diacetate was prepared from above intermediate compound (I) and hydrazinecarboxamide for next step. Analogously to General Method E, the title compound was prepared from the intermediate compound (II) 4.83 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 61. Preparation of 3-(3,4-dihydroxy-5-(hydroxymethyl)tetrahydrofuran-2-yl)-7-(4-methoxybenzamido)-4-oxo-3H-pyrazolo[4,3-d][1,2,3]triazine-5(4H)-carboxamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamoyl-4-oxo-4,5-dihydro-3H-pyrazolo[4,3-d][1,2,3]triazin-3-yl) tetrahydrofuran-3,4-diyl diacetate was prepared from the intermediate compound (I) and hydrazinecarboxamide for next step. Analogously to General Method K, the intermediate compound (III) 2-(acetoxymethyl)-5-(5-carbamoyl-7-(4-methoxy-benzamido)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoylchloride for next step. Analogously to General Method E, the title compound was prepared from the intermediate compound (III) 5.87 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound.

¹H NMR (600 MHZ, CDCl₃-d₆): δ 9.15 (s, 1H), 7.92 (d, 2H), 7.17 (d, 2H), 7.68 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.83 (s, 3H), 3.66 (m, 2H), 3.65 (s, 1H), 3.58 (s, 2H).

### Example 62. Preparation of 2-(diethoxyphosphoryl)-2-(3-(3,4-dihydroxy-5-(hydroxymethyl)tetrahydrofuran-2-yl)-7-(ethoxycarbonyl)-4-oxo-3H-pyrazolo [4,3-d][1,2,3]triazin-5(4H)-yl)acetic acid

Analogously to General Method C, the intermediate compound (I) 2-(acetoxymethyl)-5-(4-(2-ethoxy-2-oxoacetyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-oxoacetate and 5-(acetoxymethyl)tetrahy-drofuran-2,3,4-triyl triacetate for next step. Analogously to General Method J, the intermediate compound (II) 2-(3-(3,4-diacetoxy-5-(acetoxymethyl)tetrahydrofuran-2-yl)-7-(ethoxycarbonyl)-4-oxo-3H-pyrazolo[4,3-d][1,2,3]triazin-5(4H)-yl)-2-(diethoxyphosphoryl)acetic acid was prepared from the Intermediate compound (I) and 2-(diethoxyphosphoryl)-2-hydrazinylacetic acid for next step. Analogously to General Method E, the title compound was prepared from the intermediate compound (II) 6.61 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): 611.00 (s, 1H), 5.93 (d, J=7.0 Hz, 1H), 4.60 (d, J=11.5 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 4.07 (m, 4H), 3.66 (m, 2H), 3.65 (s, 1H), 3.58 (s, 2H), 1.29 (t, 9H).

### Example 63. Preparation of 2-(diethoxyphosphoryl)-2-(3-(3,4-dihydroxy-5-(hydroxymethyl)tetrahydrofuran-2-yl)-4-oxo-7-propyl-3H-pyrazolo[4,3-d][1,2,3]triazin-5(4H)-yl) acetic acid

Analogously to General Method C, the intermediate compound (I) 2-(3-(3,4-diacetoxy-5-(acetoxymethyl)tetrahydrofuran-2-yl)-4-oxo-7-propyl-3H-pyrazolo[4,3-d][1,2,3]triazin-5(4H)-yl)-2-(diethoxyphosphoryl)acetic acid was prepared from 2-(diethoxyphosphoryl)-2-(3-(3,4-dihydroxy-5-(hydroxymethyl)tetrahy-drofuran-2-yl)-4-oxo-7-propyl-3H-pyrazolo[4,3-d][1,2,3]triazin-5(4H)-yl)acetic acid and 5-(acetoxymethyl)tetrahydrofuran-2,3,4-triyl triacetate for next step. Analogously to General Method E, the title compound was prepared from the intermediate compound (I) 6.31 g (10 mmol) and the solution of methanol/NaOCH₃ 20 mL (1 mon/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): 611.00 (s, 1H), 5.93 (d, J=7.0 Hz, 1H), 4.60 (d, J=11.5 Hz, 1H), 4.51 (t, J=7.0 Hz, 1H), 4.40 (m, J=7.0 Hz, 1H), 4.28 (t, J=7.0 Hz, 1H), 4.07 (m, 4H), 3.66 (m, 2H), 3.65 (s, 1H), 3.58 (s, 2H), 2.44 (t, J=7.1 Hz, 2H), 1.65 (m, 2H), 1.29 (t, 6H), 0.90 (t, J=8.0 Hz, 3H).

### Example 64. Preparation of 2-(7-cyano-3-(3,4-dihydroxy-5-(hydroxymethyl) tetrahydrofuran-2-yl)-4-oxo-3H-pyrazolo[4,3-d][1,2,3]triazin-5(4H)-yl)-2-(diethoxyphosphoryl) acetic acid

Analogously to General Method C, the intermediate compound (I) 2-(7-cyano-3-(3,4-diacetoxy-5-(acetoxy-methyl)tetrahydrofuran-2-yl)-4-oxo-3H-pyrazolo[4,3-d][1,2,3]triazin-5(4H)-yl)-2-(diethoxyphosphoryl)acetic acid was prepared from 2-(7-cyano-4-oxo-3H-pyrazolo[4,3-d][1,2,3]triazin-5(4H)-yl)-2-(diethoxy-phosphoryl)acetic acid and 5-(acetoxymethyl)tetrahydrofuran-2,3,4-triyl triacetate for next step. Analogously to General Method E, the title compound was prepared from the intermediate compound (I) 6.14 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): 611.00 (s, 1H), 5.93 (d, J=7.0 Hz, 1H), 4.60 (d, J=11.5 Hz, 1H), 4.51 (t, J=7.0 Hz, 1H), 4.40 (m, J=7.0 Hz, 1H), 4.28 (t, J=7.0 Hz, 1H), 4.07 (m, 4H), 3.66 (m, 2H), 3.65 (s, 1H), 3.58 (s, 2H), 1.29 (t, 6H).

### Example 65. Preparation of 2-(diethoxyphosphoryl)-2-(3-(3,4-dihydroxy-5-(hydroxymethyl)tetrahydrofuran-2-yl)-4-oxo-7-(piperidin-1-yl)-3H-pyrazolo[4,3-d][1,2,3]triazin-5 (4H)-yl)acetic acid

Analogously to General Method C, the intermediate compound (I) 2-(3-(3,4-diacetoxy-5-(acetoxymethyl)tetrahydrofuran-2-yl)-4-oxo-7-(piperidin-1-yl)-3H-pyrazolo[4,3-d][1,2,3]triazin-5(4H)-yl)-2-(diethoxy-phosphoryl)acetic acid was prepared from 2-(diethoxyphosphoryl)-2-(4-oxo-7-(piperidin-1-yl)-3H-pyrazolo[4,3-d][1,2,3]triazin-5(4H)-yl)acetic acid and 5-(acetoxymethyl)tetrahydrofuran-2,3,4-triyl triacetate for next step. Analogously to General Method E, the title compound was prepared from the intermediate compound (I) 6.72 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): 611.00 (s, 1H), 5.93 (d, J=7.0 Hz, 1H), 4.60 (d, J=11.5 Hz, 1H), 4.51 (t, J=7.0 Hz, 1H), 4.40 (m, J=7.0 Hz, 1H), 4.28 (t, J=7.0 Hz, 1H), 4.07 (m, 4H), 3.66 (m, 2H), 3.65 (s, 1H), 3.58 (s, 2H), 3.11 (s, 4H), 1.65 (d, 6H), 1.29 (t, 6H).

### Example 66. Preparation of 2-(diethoxyphosphoryl)-2-(3-(3,4-dihydroxy-5-(hydroxymethyl)tetrahydrofuran-2-yl)-7-nitro-4-oxo-3H-pyrazolo[4,3-d][1,2,3]triazin-5(4H)-yl) acetic acid

Analogously to General Method C, the intermediate compound (I) 2-(3-(3,4-diacetoxy-5-(acetoxymethyl)tetrahydrofuran-2-yl)-7-nitro-4-oxo-3H-pyrazolo[4,3-d][1,2,3]triazin-5(4H)-yl)-2-(diethoxyphosphoryl)acetic acid was prepared from 2-(diethoxyphosphoryl)-2-(7-nitro-4-oxo-3H-pyrazolo[4,3-d][1,2,3]triazin-5(4H)-yl)acetic acid and 5-(acetoxymethyl)tetrahydrofuran-2,3,4-triyl triacetate for next step. Analogously to General Method E, the title compound was prepared from the intermediate compound (I) 6.34 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, DMSO-d₆): δ 11.00 (s, 1H), 6.35 (d, J=7.0 Hz, 1H), 5.42 (d, J=5.4 Hz, 1H), 5.15 (d, J=6.0 Hz, 1H), 4.60 (d, J=11.5 Hz, 1H), 4.69 (t, J=6.0 Hz, 1H), 4.51 (m, 1H), 4.21 (m, 1H), 4.07 (m, 4H), 3.91 (m, 1H), 3.57 (m, 1H), 3.42 (m, 1H), 1.29 (t, 6H).

### Example 67. Preparation of ethyl 5-carbamimidoyl-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate

Analogously to General Method C, the intermediate compound (I) 2-(acetoxymethyl)-5-(4-(2-ethoxy-1-imino-2-oxoethyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-iminoacetate and 5-(acetoxymethyl)tetrahydrofuran-2,3,4-triyl triacetate for next step. Analogously to General Method J, the intermediate compound (II) 2-(acetoxymethyl)-5-(5-carbamimidoyl-7-(ethoxycarbonyl)-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarboximidamide for next step. Analogously to General Method E, the title compound was prepared from the intermediate compound (II) 5.09 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.58 (s, 2H), 1.29 (t, J=8.0 Hz, 3H).

### Example 68. Preparation of 3-(3,4-dihydroxy-5-(hydroxymethyl)tetrahydrofuran-2-yl)-4-oxo-7-propyl-3H-pyrazolo [4,3-d][1,2,3]triazine-5(4H)-carboximidamide

Analogously to General Method C, the intermediate compound (I) 3-(3,4-dihydroxy-5-(hydroxymethyl)tetrahydrofuran-2-yl)-6-(1-iminobutyl)-1,2,3-triazine-4,5(3H,6H)-dione was prepared from 6-(1-imino-butyl)-1,2,3-triazine-4,5(3H,6H)-dione and 5-(acetoxymethyl)tetrahydrofuran-2,3,4-triyl triacetate for next step. Analogously to General Method J, the intermediate compound (II) 3-(3,4-dihydroxy-5-(hydroxyl-methyl)tetrahydrofuran-2-yl)-4-oxo-7-propyl-3H-pyrazolo[4,3-d][1,2,3]triazine-5(4H)-carboximidamide was prepared from above intermediate compound (I) and hydrazinecarboximidamide for next step. Analogously to General Method E, the title compound was prepared from the intermediate compound (II) 3.53 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2H), 1.65 (m, 2H), 0.90 (t, J=8.0 Hz, 3H).

### Example 69. Preparation of 7-amino-3-(3,4-dihydroxy-5-(hydroxymethyl) tetrahydrofuran-2-yl)-4-oxo-3H-pyrazolo[4,3-d][1,2,3]triazine-5(4H)-carboximidamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamimidoyl-4-oxo-4,5-dihydro-3H-pyrazolo[4,3-d][1,2,3]triazin-3-yl) tetrahydrofuran-3,4-diyl diacetate was prepared from above intermediate compound (I) and hydrazinecarboximidamide for next step. Analogously to General Method E, the title compound was prepared from the intermediate compound (II) 4.52 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 7.74 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 70. Preparation of 7-cyano-3-(3,4-dihydroxy-5-(hydroxymethyl) tetrahydrofuran-2-yl)-4-oxo-3H-pyrazolo[4,3-d][1,2,3]triazine-5(4H)-carboximidamide

Analogously to General Method C, the intermediate compound (I) 2-(acetoxymethyl)-5-(4-(cyano (imino)methyl)-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbimidoyl cyanide and 5-(acetoxymethyl)tetrahydrofuran-2,3,4-triyl triacetate for next step. Analogously to General Method J, the intermediate compound (II) 2-(acetoxy-methyl)-5-(5-carbamimidoyl-7-cyano-4-oxo-4,5-dihydro-3H-pyrazolo[4,3-d][1,2,3]triazin-3-yl)tetrahy-drofuran-3,4-diyl diacetate was prepared from above intermediate compound (I) and hydrazinecarboxi-midamide for next step. Analogously to General Method E, the title compound was prepared from the intermediate compound (II) 4.62 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 71. Preparation of 3-(3,4-dihydroxy-5-(hydroxymethyl)tetrahydrofuran-2-yl)-4-oxo-7-(piperidin-1-yl)-3H-pyrazolo[4,3-d][1,2,3]triazine-5(4H)-carboximidamide

Analogously to General Method C, the intermediate compound (I) 2-(acetoxymethyl)-5-(4-(imino (piperidin-1-yl)methyl)-5,6-dioxo-5,6-dihydro-1,2,3-triazin-1(4H)-yl)tetrahydrofuran-3,4-diyl diacetate was prepared from 6-(imino(piperidin-1-yl)methyl)-1,2,3-triazine-4,5(3H,6H)-dione and 5-(acetoxymethyl)tetrahy-drofuran-2,3,4-triyl triacetate for next step. Analogously to General Method J, the intermediate compound (II) 2-(acetoxymethyl)-5-(5-carbamimidoyl-4-oxo-7-(piperidin-1-yl)-4,5-dihydro-3H-pyrazolo[4,3-d][1,2,3]triazin-3-yl)tetrahydrofuran-3,4-diyl diacetate was prepared from above intermediate compound (I) and hydrazinecarboximidamide for next step. Analogously to General Method E, the title compound was prepared from the intermediate compound (II) 5.20 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 3.11 (s, 4H), 1.65 (d, 6H).

### Example 72. Preparation of 3-(3,4-dihydroxy-5-(hydroxymethyl)tetrahydrofuran-2-yl)-7-nitro-4-oxo-3H-pyrazolo[4,3-d][1,2,3]triazine-5(4H)-carboximidamide

Analogously to General Method C, the intermediate compound (I) 2-(acetoxymethyl)-5-(4-(imino (nitro)methyl)-5,6-dioxo-5,6-dihydro-1,2,3-triazin-1(4H)-yl)tetrahydrofuran-3,4-diyl diacetate was prepared from 6-(imino(nitro)methyl)-1,2,3-triazine-4,5(3H,6H)-dione and 5-(acetoxymethyl) tetrahydrofuran-2,3,4-triyl triacetate for next step. Analogously to General Method J, the intermediate compound (II) 2-(acetoxymethyl)-5-(5-carbamimidoyl-7-nitro-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarboximidamide for next step. Analogously to General Method E, the title compound was prepared from the intermediate compound (II) 4.82 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 73. Preparation of ethyl 5-carbamothioyl-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate

Analogously to General Method C, the intermediate compound (I) 2-(acetoxymethyl)-5-(4-(2-ethoxy-1-imino-2-oxoethyl)-5,6-dioxo-5,6-dihydro-1,2,3-triazin-1(4H)-yl)tetrahydrofuran-3,4-diyl diacetate was prepared from ethyl 2-(5,6-dioxo-1,4,5,6-tetrahydro-1,2,3-triazin-4-yl)-2-iminoacetate and 5-(acetoxymethyl)tetrahydrofuran-2,3,4-triyl triacetate for next step. Analogously to General Method J, the intermediate compound (II) 2-(acetoxymethyl)-5-(5-carbamothioyl-7-(ethoxycarbonyl)-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarbothioamide for next step. Analogously to General Method E, the title compound was prepared from the intermediate compound (II) 5.26 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (t, J=7.0 Hz, 1H), 3.66 (m, 2H), 3.65 (s, 1H), 3.58 (s, 2H), 1.29 (t, J=8.0 Hz, 3H).

### Example 74. Preparation of 3-(3,4-dihydroxy-5-(hydroxymethyl)tetrahydrofuran-2-yl)-4-oxo-7-propyl-3H-pyrazolo[4,3-d][1,2,3]triazine-5(4H)-carbothioamide

Analogously to General Method C, the intermediate compound (I) 2-(acetoxymethyl)-5-(4-(1-iminobutyl)-5,6-dioxo-5,6-dihydro-1,2,3-triazin-1(4H)-yl)tetrahydrofuran-3,4-diyl diacetate was prepared from 6-(1-iminobutyl)-1,2,3-triazine-4,5(3H,6H)-dione and 5-(acetoxymethyl)tetrahydrofuran-2,3,4-triyl triacetate for next step. Analogously to General Method J, the intermediate compound (II) 2-(acetoxymethyl)-5-(5-carbamothioyl-4-oxo-7-propyl-4,5-dihydro-3H-pyrazolo[4,3-d][1,2,3]triazin-3-yl)tetrahydrofuran-3,4-diyl diacetate was prepared from above intermediate compound (I) and hydrazinecarbothioamide for next step. Analogously to General Method E, the title compound was prepared from the intermediate compound (II) 5.26 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H), 2.44 (t, J=7.1 Hz, 2H), 1.65 (m, 2H), 0.90 (t, J=8.0 Hz, 3H).

### Example 75. Preparation of 7-amino-3-(3,4-dihydroxy-5-(hydroxymethyl) tetrahydrofuran-2-yl)-4-oxo-3H-pyrazolo[4,3-d][1,2,3]triazine-5(4H)-carbothioamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamothioyl-4-oxo-4,5-dihydro-3H-pyrazolo[4,3-d][1,2,3]triazin-3-yl) tetrahydrofuran-3,4-diyl diacetate was prepared from above intermediate compound (I) and hydrazinecarbothioamide for next step. Analogously to General Method E, the title compound was prepared from the intermediate compound (II) 4.69 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 7.74 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 76. Preparation of N-(5-carbamothioyl-3-(3,4-dihydroxy-5-(hydroxymethyl) tetrahydrofuran-2-yl)-4-oxo-4,5-dihydro-3H-pyrazolo[4,3-d][1,2,3]triazin-7-yl)-3-(dimethylamino) propanamide

Analogously to General Method C, the intermediate compound (I) 2-(acetoxymethyl)-5-(4-cyano-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonitrile and 5-(acetoxymethyl)tetrahydrofuran-2,3,4-triyl triacetate for next step. Analogously to General Method J, the intermediate compound (II) 2-(acetoxymethyl)-5-(7-amino-5-carbamothioyl-4-oxo-4,5-dihydro-3H-pyrazolo[4,3-d][1,2,3]triazin-3-yl) tetrahydrofuran-3,4-diyl diacetate was prepared from the intermediate compound (I) and hydrazinecarbothioamide for next step. Analogously to General Method K, the intermediate compound (III) 2-(acetoxymethyl)-5-(5-carbamothioyl-7-(3-(dimethylamino)propanamido)-4-oxo-4,5-dihydro-3H-pyrazolo[4,3-d][1,2,3]triazin-3-yl)tetrahydrofuran-3,4-diyl diacetate was prepared from above intermediate compound (II) and 3-(dimethylamino)propanoyl-chloride for next step. Analogously to General Method E, the title compound was prepared from the intermediate compound (III) 5.68 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 8.56 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (t, J=7.1 Hz, 2H), 3.60 (s, 1H), 3.58 (s, 2H), 2.84 (s, 6H), 2.50 (t, J=7.1 Hz, 2H).

### Example 77. Preparation of 7-cyano-3-(3,4-dihydroxy-5-(hydroxymethyl) tetrahydrofuran-2-yl)-4-oxo-3H-pyrazolo[4,3-d][1,2,3]triazine-5(4H)-carbothioamide

Analogously to General Method C, the intermediate compound (I) 2-(acetoxymethyl)-5-(4-(cyanocarbonyl)-5,6-dioxo-5,6-dihydro-1,2,3-triazin-1(4H)-yl)tetrahydrofuran-3,4-diyl diacetate was prepared from 5,6-dioxo-1,4,5,6-tetrahydro-1,2,3-triazine-4-carbonyl cyanide and 5-(acetoxymethyl) tetrahydrofuran-2,3,4-triyl triacetate for next step. Analogously to General Method J, the intermediate compound (II) 2-(acetoxymethyl)-5-(5-carbamothioyl-7-cyano-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarbothioamide for next step. Analogously to General Method E, the title compound was prepared from the intermediate compound (II) 4.79 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 78. Preparation of 3-(3,4-dihydroxy-5-(hydroxymethyl)tetrahydrofuran-2-yl)-7-nitro-4-oxo-3H-pyrazolo[4,3-d][1,2,3]triazine-5(4H)-carbothioamide

Analogously to General Method C, the intermediate compound (I) 2-(acetoxymethyl)-5-(4-(nitrocarbonyl)-5,6-dioxo-5,6-dihydro-1,2,3-triazin-1(4H)-yl)tetrahydrofuran-3,4-diyl diacetate was prepared from 6-(nitrocarbonyl)-1,2,3-triazine-4,5(3H,6H)-dione and 5-(acetoxymethyl)tetrahydrofuran-2,3,4-triyl triacetate for next step. Analogously to General Method J, the intermediate compound (II) 2-(acetoxymethyl)-5-(5-carbamothioyl-7-nitro-4-oxo-4,5-dihydro-3H-pyrazolo[4,3-d][1,2,3]triazin-3-yl)tetrahydrofuran-3,4-diyl diacetate was prepared from above intermediate compound (I) and hydrazinecarbothioamide for next step. Analogously to General Method E, the title compound was prepared from the intermediate compound (II) 4.99 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 1H), 3.58 (s, 2H).

### Example 79. Preparation of 5-hydroxy-3-(5-(hydroxymethyl)tetrahydrofuran-2-yl)-7-propyl-3H-pyrazolo[4,3-d][1,2,3]triazin-4(5H)-one

Analogously to General Method C, the intermediate compound (I) (5-(4-butyryl-5,6-dioxo-5,6-dihydro-1,2,3-triazin-1(4H)-yl)tetrahydrofuran-2-yl)methyl acetate was prepared from 6-butyryl-1,2,3-triazine-4,5(3H,6H)-dione and 5-(acetoxymethyl)tetrahydrofuran-2-yl acetate for next step. Analogously to General Method J, the intermediate compound (II) (5-(5-hydroxy-4-oxo-7-propyl-4,5-dihydro-3H-pyrazolo[4,3-d][1,2,3]triazin-3-yl)tetrahydrofuran-2-yl)methyl acetate was prepared from above intermediate compound (I) and hydroxyhydrazine for next step. Analogously to General Method E, the title compound was prepared from the intermediate compound (II) 3.37 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.85 (t, J=7.0 Hz, 1H), 3.83 (m, J=7.0 Hz, 1H), 3.66 (m, 2H), 3.65 (s, 1H), 2.44 (t, J=7.1 Hz, 2H), 2.07 (m, 2H), 2.00 (s, 1H), 1.81 (m, 2H), 1.65 (m, 2H), 0.90 (t, J=8.0 Hz, 3H).

### Example 80. Preparation of 3-(5-(hydroxymethyl)tetrahydrofuran-2-yl)-5-methyl-7-propyl-3H-pyrazolo[4,3-d][1,2,3]triazin-4(5H)-one

Analogously to General Method C, the intermediate compound (I) (5-(5-methyl-4-oxo-7-propyl-4,5-dihydro-3H-pyrazolo[4,3-d][1,2,3]triazin-3-yl)tetrahydrofuran-2-yl)methyl acetate was prepared from 5-methyl-7-propyl-3H-pyrazolo[4,3-d][1,2,3]triazin-4(5H)-one and 5-(acetoxymethyl)tetrahydrofuran-2-ylacetate for next step. Analogously to General Method E, the title compound was prepared from the intermediate compound (I) 5.15 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.85 (t, J=7.0 Hz, 1H), 3.95 (s, 3H), 3.83 (m, J=7.0 Hz, 1H), 3.66 (m, 2H), 3.65 (s, 1H), 2.44 (t, J=7.1 Hz, 2H), 2.07 (m, 2H), 1.81 (m, 2H), 1.65 (m, 2H), 0.90 (t, J=8.0 Hz, 3H).

### Example 81. Preparation of 2-(diethoxyphosphoryl)-2-((5-(hydroxymethyl)-3-(5-(hydroxymethyl)tetrahydrofuran-2-yl)-4-oxo-4,5-dihydro-3H-pyrazolo[4,3-d][1,2,3]triazin-7-yl) amino)acetic acid

Analogously to General Method C, the intermediate compound (I) (5-(4-cyano-5,6-dioxo-5,6-dihydro-1,2,3-triazin-1(4H)-yl)tetrahydrofuran-2-yl)methyl acetate was prepared from 5,6-dioxo-1,4,5,6-tetrahydro-1,2,3-triazine-4-carbonitrile and 5-(acetoxymethyl)tetrahydrofuran-2-yl acetate for next step. Analogously to General Method J, the intermediate compound (II) (5-(7-amino-5-(hydroxymethyl)-4-oxo-4,5-dihydro-3H-pyrazolo[4,3-d][1,2,3]triazin-3-yl)tetrahydrofuran-2-yl)methyl acetate was prepared from the intermediate compound (I) and hydrazinylmethanol for next step. Analogously to General Method K, the intermediate compound (III) 2-((3-(5-(acetoxymethyl) tetrahydrofuran-2-yl)-5-(hydroxymethyl)-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 5.18 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.97 (s, 2H), 5.85 (t, J=7.0 Hz, 1H), 4.07 (m, 4H), 4.00 (s, 1H), 3.83 (m, J=7.0 Hz, 1H), 3.66 (m, 2H), 3.65 (s, 2H), 3.50 (d, J=11.5 Hz, 1H), 2.07 (m, 2H), 1.81 (m, 2H), 1.29 (t, 6H).

### Example 82. Preparation of 2-(diethoxyphosphoryl)-2-(3-(5-(hydroxymethyl) tetrahydrofuran-2-yl)-4-oxo-7-propyl-3H-pyrazolo[4,3-d][1,2,3]triazin-5(4H)-yl)acetic acid

Analogously to General Method C, the intermediate compound (I) 2-(3-(5-(acetoxymethyl) tetrahydrofuran-2-yl)-4-oxo-7-propyl-3H-pyrazolo[4,3-d][1,2,3]triazin-5(4H)-yl)-2-(diethoxyphosphoryl) acetic acid was prepared from 2-(diethoxyphosphoryl)-2-(4-oxo-7-propyl-3H-pyrazolo[4,3-d][1,2,3]triazin-5(4H)-yl)acetic acid and 5-(acetoxymethyl)tetrahydrofuran-2-yl acetate for next step. Analogously to General Method E, the title compound was prepared from the intermediate compound (I) 5.15 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.85 (t, J=7.0 Hz, 1H), 4.60 (d, J=11.5 Hz, 1H), 4.07 (m, 4H), 3.83 (m, J=7.0 Hz, 1H), 3.66 (m, 2H), 3.65 (s, 1H), 2.44 (t, J=7.1 Hz, 2H), 2.07 (m, 2H), 1.81 (m, 2H), 1.65 (m, 2H), 1.29 (t, 6H), 0.90 (t, J=8.0 Hz, 3H).

### Example 83. Preparation of 2-(7-amino-3-(5-(hydroxymethyl)tetrahydrofuran-2-yl)-4-oxo-3H-pyrazolo[4,3-d][1,2,3]triazin-5(4H)-yl)-2-(diethoxyphosphoryl)acetic acid

Analogously to General Method C, the intermediate compound (I) (5-(4-cyano-5,6-dioxo-5,6-dihydro-1,2,3-triazin-1(4H)-yl)tetrahydrofuran-2-yl)methyl acetate was prepared from 6-butyryl-1,2,3-triazine-4,5(3H,6H)-dione and 5-(acetoxymethyl)tetrahydrofuran-2,3,4-triyl triacetate for next step. Analogously to General Method J, the intermediate compound (II) 2-(3-(5-(acetoxymethyl) tetrahydrofuran-2-yl)-7-amino-4-oxo-3H-pyrazolo[4,3-d][1,2,3]triazin-5(4H)-yl)-2-(diethoxyphosphoryl) acetic acid was prepared from above intermediate compound (I) and 2-(diethoxyphosphoryl)-2-hydrazinylacetic acid for next step. Analogously to General Method E, the title compound was prepared from the intermediate compound (II) 4.88 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.74 (s, 2H), 5.85 (t, J=7.0 Hz, 1H), 4.60 (d, J=11.5 Hz, 1H), 4.07 (m, 4H), 3.83 (m, J=7.0 Hz, 1H), 3.66 (m, 2H), 3.65 (s, 1H), 2.07 (m, 2H), 1.81 (m, 2H), 1.29 (t, 6H).

### Example 84. Preparation of 2-(7-cyano-3-(5-(hydroxymethyl)tetrahydrofuran-2-yl)-4-oxo-3H-pyrazolo[4,3-d][1,2,3]triazin-5(4H)-yl)-2-(diethoxyphosphoryl)acetic acid

Analogously to General Method C, the intermediate compound (I) 2-(3-(5-(acetoxymethyl) tetrahydrofuran-2-yl)-7-cyano-4-oxo-3H-pyrazolo[4,3-d][1,2,3]triazin-5(4H)-yl)-2-(diethoxyphosphoryl) acetic acid was prepared from 2-(7-cyano-4-oxo-3H-pyrazolo[4,3-d][1,2,3]triazin-5(4H)-yl)-2-(diethoxyphosphoryl)acetic acid and 5-(acetoxymethyl)tetrahydrofuran-2-yl acetate for next step. Analogously to General Method E, the title compound was prepared from the intermediate compound (I) 4.98 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.85 (t, J=7.0 Hz, 1H), 4.60 (d, J=11.5 Hz, 1H), 4.07 (m, 4H), 3.83 (m, J=7.0 Hz, 1H), 3.66 (m, 2H), 3.65 (s, 1H), 2.07 (m, 2H), 1.81 (m, 2H), 1.29 (t, 6H).

### Example 85. Preparation of 3-(5-(hydroxymethyl)tetrahydrofuran-2-yl)-4-oxo-7-propyl-3H-pyrazolo[4,3-d][1,2,3]triazine-5(4H)-carboximidamide

Analogously to General Method C, the intermediate compound (I) (5-(4-butyryl-5,6-dioxo-5,6-dihydro-1,2,3-triazin-1(4H)-yl)tetrahydrofuran-2-yl)methylacetate was prepared from 6-butyryl-1,2,3-triazine-4,5(3H,6H)-dione and 5-(acetoxymethyl)tetrahydrofuran-2,3,4-triyl triacetate for next step. Analogously to General Method J, the intermediate compound (II) (5-(5-carbamimidoyl-4-oxo-7-propyl-4,5-dihydro-3H-pyrazolo[4,3-d][1,2,3]triazin-3-yl)tetrahydrofuran-2-yl)methylacetate was prepared from above intermediate compound (I) and hydrazinecarboximidamide for next step. Analogously to General Method E, the title compound was prepared from the intermediate compound (II) 3.63 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 8.03 (s, 1H), 5.85 (t, J=7.0 Hz, 1H), 3.83 (m, J=7.0 Hz, 1H), 3.66 (m, 2H), 3.65 (s, 1H), 2.44 (t, J=7.1 Hz, 2H), 2.07 (m, 2H), 1.81 (m, 2H), 1.65 (m, 2H), 0.90 (t, J=8.0 Hz, 3H).

### Example 86. Preparation of 3-(5-(hydroxymethyl)tetrahydrofuran-2-yl)-4-oxo-7-propyl-3H-pyrazolo[4,3-d][1,2,3]triazine-5(4H)-carbothioamide

Analogously to General Method C, the intermediate compound (I) (5-(4-(1-iminobutyl)-5,6-dioxo-5,6-dihydro-1,2,3-triazin-1(4H)-yl)tetrahydrofuran-2-yl)methyl acetate was prepared from 6-(1-iminobutyl)-1,2,3-triazine-4,5(3H,6H)-dione and 5-(acetoxymethyl)tetrahydrofuran-2-yl acetate for next step. Analogously to General Method J, the intermediate compound (II) (5-(5-carbamothioyl-4-oxo-7-propyl-4,5-dihydro-3H-pyrazolo[4,3-d][1,2,3]triazin-3-yl)tetrahydrofuran-2-yl)methyl acetate was prepared from above intermediate compound (I) and hydrazinecarbothioamide for next step. Analogously to General Method E, the title compound was prepared from the intermediate compound (II) 3.80 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 8.56 (s, 2H), 5.85 (t, J=7.0 Hz, 1H), 3.83 (m, J=7.0 Hz, 1H), 3.66 (m, 2H), 3.65 (s, 1H), 2.44 (t, J=7.1 Hz, 2H), 2.07 (m, 2H), 1.81 (m, 2H), 1.65 (m, 2H), 0.90 (t, J=8.0 Hz, 3H).

### Example 87. Preparation of 2-(diethoxyphosphoryl)-2-((5-hydroxy-3-(2-(hydroxymethyl)-1,3-oxathiolan-5-yl)-4-oxo-4,5-dihydro-3H-pyrazolo[4,3-d][1,2,3]triazin-7-yl) amino)acetic acid

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5,6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-hydroxy-4-oxo-4,5-dihydro-3H-pyrazolo[4,3-d][1,2,3]triazin-3-yl)-1,3-oxathiolan-2-yl)methyl acetate was prepared from the intermediate compound (I) and hydroxyhydrazine for next step. Analogously to General Method K, the intermediate compound (III) 2-((3-(2-(acetoxymethyl)-1,3-oxathiolan-5-yl)-5-hydroxy-4-oxo-4,5-dihydro-3H-pyrazolo[4,3-d][1,2,3]triazin-7-yl)amino)-2-(diethoxyphosphoryl) acetic acid was prepared from above intermediate compound (II) and 2-chloro-2-(diethoxyphosphoryl) acetic acid for next step. Analogously to General Method E, the title compound was prepared from the intermediate compound (III) 5.22 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.22 (t, J=7.0 Hz, 1H), 4.07 (m, 4H), 4.03 (m, 2H), 4.00 (s, 1H), 3.97 (m, J=7.0 Hz, 1H), 3.65 (s, 1H), 3.50 (d, J=11.5 Hz, 1H), 2.83 (m, 2H), 2.00 (s, 1H), 1.29 (t, 6H).

### Example 88. Preparation of 5-hydroxy-3-(2-(hydroxymethyl)-1,3-oxathiolan-5-yl)-7-propyl-3H-pyrazolo[4,3-d][1,2,3]triazin-4(5H)-one

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-hydroxy-4-oxo-7-propyl-4,5-dihydro-3H-pyrazolo[4,3-d][1,2,3]triazin-3-yl)-1,3-oxathiolan-2-yl)methylacetate was prepared from above intermediate compound (I) and hydroxyhydrazine for next step. Analogously to General Method E, the title compound was prepared from the intermediate compound (II) 3.55 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.22 (t, J=7.0 Hz, 1H), 4.03 (m, 2H), 3.97 (m, J=7.0 Hz, 1H), 3.65 (s, 1H), 2.83 (m, 2H), 2.44 (t, J=7.1 Hz, 2H), 2.00 (s, 1H), 1.65 (m, 2H), 0.90 (t, J=8.0 Hz, 3H).

### Example 89. Preparation of 7-amino-5-hydroxy-3-(2-(hydroxymethyl)-1,3-oxathiolan-5-yl)-3H-pyrazolo[4,3-d][1,2,3]triazin-4(5H)-one

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5,6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-hydroxy-4-oxo-4,5-dihydro-3H-pyrazolo[4,3-d][1,2,3]triazin-3-yl)-1,3-oxathiolan-2-yl)methyl acetate was prepared from above intermediate compound (I) and hydroxyhydrazine for next step. Analogously to General Method E, the title compound was prepared from the intermediate compound (II) 3.28 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.74 (s, 2H), 5.22 (t, J=7.0 Hz, 1H), 4.03 (m, 2H), 3.97 (m, J=7.0 Hz, 1H), 3.65 (s, 1H), 2.83 (m, 2H), 2.00 (s, 1H).

### Example 90. Preparation of 2-(diethoxyphosphoryl)-2-((3-(2-(hydroxymethyl)-1,3-oxathiolan-5-yl)-5-methyl-4-oxo-4,5-dihydro-3H-pyrazolo[4,3-d][1,2,3]triazin-7-yl)amino)acetic acid

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5, 6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-methyl-4-oxo-4,5-dihydro-3H-pyrazolo[4,3-d][1,2,3]triazin-3-yl)-1,3-oxathiolan-2-yl)methyl acetate was prepared from the intermediate compound (I) and methylhydrazine for next step. Analogously to General Method K, the intermediate compound (III) 2-((3-(2-(acetoxymethyl)-1,3-oxathiolan-5-yl)-5-methyl-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 5.20 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.22 (t, J=7.0 Hz, 1H), 4.07 (m, 4H), 4.03 (m, 2H), 4.00 (s, 1H), 3.97 (m, J=7.0 Hz, 1H), 3.95 (s, 3H), 3.65 (s, 1H), 3.50 (d, J=11.5 Hz, 1H), 2.83 (m, 2H), 1.29 (t, 6H).

### Example 91. Preparation of 3-(2-(hydroxymethyl)-1,3-oxathiolan-5-yl)-5-methyl-7-propyl-3H-pyrazolo[4,3-d][1,2,3]triazin-4(5H)-one

Analogously to General Method C, the intermediate compound (I) (5-(5-methyl-4-oxo-7-propyl-4,5-dihydro-3H-pyrazolo[4,3-d][1,2,3]triazin-3-yl)-1,3-oxathiolan-2-yl)methyl acetate was prepared from 5-methyl-7-propyl-3H-pyrazolo[4,3-d][1,2,3]triazin-4(5H)-one and (5-acetoxy-1,3-oxathiolan-2-yl) methyl acetate for next step. Analogously to General Method E, the title compound was prepared from the intermediate compound (I) 3.53 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.22 (t, J=7.0 Hz, 1H), 4.03 (m, 2H), 3.97 (m, J=7.0 Hz, 1H), 3.95 (s, 3H), 3.65 (s, 1H), 2.83 (m, 2H), 2.44 (t, J=7.1 Hz, 2H), 1.65 (m, 2H), 0.90 (t, J=8.0 Hz, 3H).

### Example 92. Preparation of 3-(2-(hydroxymethyl)-1,3-oxathiolan-5-yl)-5-methyl-4-oxo-4,5-dihydro-3H-pyrazolo[4,3-d][1,2,3]triazine-7-carbonitrile

Analogously to General Method C, the intermediate compound (I) (5-(7-cyano-5-methyl-4-oxo-4,5-dihydro-3H-pyrazolo[4,3-d][1,2,3]triazin-3-yl)-1,3-oxathiolan-2-yl)methyl acetate was prepared from 5-methyl-4-oxo-4,5-dihydro-3H-pyrazolo[4,3-d][1,2,3]triazine-7-carbonitrile and (5-acetoxy-1,3-oxathiolan-2-yl)methyl acetate for next step. Analogously to General Method E, the title compound was prepared from the intermediate compound (I) 3.36 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.22 (t, J=7.0 Hz, 1H), 4.03 (m, 2H), 3.97 (m, J=7.0 Hz, 1H), 3.95 (s, 3H), 3.65 (s, 1H), 2.83 (m, 2H).

### Example 93. Preparation of 3-(2-(hydroxymethyl)-1,3-oxathiolan-5-yl)-5-methyl-7-nitro-3H-pyrazolo[4,3-d][1,2,3]triazin-4(5H)-one

Analogously to General Method C, the intermediate compound (I) (5-(5-methyl-7-nitro-4-oxo-4,5-dihydro-3H-pyrazolo[4,3-d][1,2,3]triazin-3-yl)-1,3-oxathiolan-2-yl)methyl acetate was prepared from 5-methyl-7-nitro-3H-pyrazolo[4,3-d][1,2,3]triazin-4(5H)-one and (5-acetoxy-1,3-oxathiolan-2-yl)methyl acetate for next step. Analogously to General Method E, the title compound was prepared from the intermediate compound (I) 3.56 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.22 (t, J=7.0 Hz, 1H), 4.03 (m, 2H), 3.97 (m, J=7.0 Hz, 1H), 3.95 (s, 3H), 3.65 (s, 1H), 2.83 (m, 2H).

### Example 94. Preparation of 5-(hydroxymethyl)-3-(2-(hydroxymethyl)-1,3-oxathiolan-5-yl)-7-propyl-3H-pyrazolo[4,3-d][1,2,3]triazin-4(5H)-one

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-(hydroxymethyl)-4-oxo-7-propyl-4,5-dihydro-3H-pyrazolo[4,3-d][1,2,3]triazin-3-yl)-1,3-oxathiolan-2-yl)methyl acetate was prepared from above intermediate compound (I) and hydrazinylmethanol for next step. Analogously to General Method E, the title compound was prepared from the intermediate compound (II) 3.28 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.97 (s, 2H), 5.22 (t, J=7.0 Hz, 1H), 4.03 (m, 2H), 3.97 (m, J=7.0 Hz, 1H), 3.65 (s, 2H), 2.83 (m, 2H), 2.44 (t, J=7.1 Hz, 2H), 1.65 (m, 2H), 0.90 (t, J=8.0 Hz, 3H).

### Example 95. Preparation of 2-(3-(2-(hydroxymethyl)-1,3-oxathiolan-5-yl)-4-oxo-7-propyl-3H-pyrazolo[4,3-d][1,2,3]triazin-5(4H)-yl)acetamide

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-(2-amino-2-oxoethyl)-4-oxo-7-propyl-4,5-dihydro-3H-pyrazolo[4,3-d][1,2,3]triazin-3-yl)-1,3-oxathiolan-2-yl)methyl acetate was prepared from above intermediate compound (I) and 2-hydrazinylacetamide for next step. Analogously to General Method E, the title compound was prepared from the intermediate compound (II) 3.96 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.16 (s, 2H), 5.60 (s, 2H), 5.22 (t, J=7.0 Hz, 1H), 4.03 (m, 2H), 3.97 (m, J=7.0 Hz, 1H), 3.65 (s, 1H), 2.83 (m, 2H), 2.44 (t, J=7.1 Hz, 2H), 1.65 (m, 2H), 0.90 (t, J=8.0 Hz, 3H).

### Example 96. Preparation of 2-(7-amino-3-(2-(hydroxymethyl)-1,3-oxathiolan-5-yl)-4-oxo-3H-pyrazolo[4,3-d][1,2,3]triazin-5(4H)-yl)acetamide

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5,6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-(2-amino-2-oxoethyl)-4-oxo-4,5-dihydro-3H-pyrazolo[4,3-d][1,2,3]triazin-3-yl)-1,3-oxathiolan-2-yl)methyl acetate was prepared from above intermediate compound (I) and 2-hydrazinylacetamide for next step. Analogously to General Method E, the title compound was prepared from the intermediate compound (II) 3.69 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 7.16 (s, 2H), 5.60 (s, 2H), 5.22 (t, J=7.0 Hz, 1H), 4.03 (m, 2H), 3.97 (m, J=7.0 Hz, 1H), 3.65 (s, 1H), 2.83 (m, 2H).

### Example 97. Preparation of 3-(2-(hydroxymethyl)-1,3-oxathiolan-5-yl)-4-oxo-7-propyl-3H-pyrazolo[4,3-d][1,2,3]triazine-5(4H)-carboxamide

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-carbamoyl-4-oxo-7-propyl-4,5-dihydro-3H-pyrazolo[4,3-d][1,2,3]triazin-3-yl)-1,3-oxathiolan-2-yl)methyl acetate was prepared from above intermediate compound (I) and hydrazinecarboxamide for next step. Analogously to General Method E, the title compound was prepared from the intermediate compound (II) 3.82 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 7.68 (s, 2H), 5.22 (t, J=7.0 Hz, 1H), 4.03 (m, 2H), 3.97 (m, J=7.0 Hz, 1H), 3.65 (s, 1H), 2.83 (m, 2H), 2.44 (t, J=7.1 Hz, 2H), 1.65 (m, 2H), 0.90 (t, J=8.0 Hz, 3H).

### Example 98. Preparation of 7-amino-3-(2-(hydroxymethyl)-1,3-oxathiolan-5-yl)-4-oxo-3H-pyrazolo[4,3-d][1,2,3]triazine-5(4H)-carboxamide

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5,6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-carbamoyl-4-oxo-4,5-dihydro-3H-pyrazolo[4,3-d][1,2,3]triazin-3-yl)-1,3-oxathiolan-2-yl)methyl acetate was prepared from above intermediate compound (I) and hydrazinecarboxamide for next step. Analogously to General Method E, the title compound was prepared from the intermediate compound (II) 3.55 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 7.68 (s, 2H), 5.22 (t, J=7.0 Hz, 1H), 4.03 (m, 2H), 3.97 (m, J=7.0 Hz, 1H), 3.65 (s, 1H), 2.83 (m, 2H).

### Example 99. Preparation of 7-(3-(dimethylamino)propanamido)-3-(2-(hydroxymethyl)-1,3-oxathiolan-5-yl)-4-oxo-3H-pyrazolo[4,3-d][1,2,3]triazine-5(4H)-carboxamide

Analogously to General Method C, the intermediate compound (I) (5-(4-cyano-5,6-dioxo-5,6-dihydro-1,2,3-triazin-1(4H)-yl)-1,3-oxathiolan-2-yl)methyl acetate was prepared from 5,6-dioxo-1,4,5,6-tetrahydro-1,2,3-triazine-4-carbonitrile and (5-acetoxy-1,3-oxathiolan-2-yl)methyl acetate for next step. Analogously to General Method J, the intermediate compound (II) (5-(7-amino-5-methyl-4-oxo-4,5-dihydro-3H-pyrazolo[4,3-d][1,2,3]triazin-3-yl)-1,3-oxathiolan-2-yl)methyl acetate was prepared from the intermediate compound (I) and methylhydrazine for next step. Analogously to General Method K, the intermediate compound (III) 2-((3-(2-(acetoxymethyl)-1,3-oxathiolan-5-yl)-5-methyl-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 5.20 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 7.68 (s, 2H), 5.22 (t, J=7.0 Hz, 1H), 4.03 (m, 2H), 3.97 (m, J=7.0 Hz, 1H), 3.65 (t, J=7.1 Hz, 2H), 3.60 (s, 1H), 2.84 (s, 6H), 2.83 (m, 2H), 2.50 (t, J=7.1 Hz, 2H).

### Example 100. Preparation of 3-(2-(hydroxymethyl)-1,3-oxathiolan-5-yl)-4-oxo-7-propyl-3H-pyrazolo[4,3-d][1,2,3]triazine-5(4H)-carboximidamide

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-carbamimidoyl-4-oxo-7-propyl-4,5-dihydro-3H-pyrazolo[4,3-d][1,2,3]triazin-3-yl)-1,3-oxathiolan-2-yl)methyl acetate was prepared from above intermediate compound (I) and hydrazinecarboximidamide for next step. Analogously to General Method E, the title compound was prepared from the intermediate compound (II) 3.81 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 8.56 (s, 2H), 8.03 (s, 1H), 5.22 (t, J=7.0 Hz, 1H), 4.03 (m, 2H), 3.97 (m, J=7.0 Hz, 1H), 3.65 (s, 1H), 2.83 (m, 2H), 2.44 (t, J=7.1 Hz, 2H), 1.65 (m, 2H), 0.90 (t, J=8.0 Hz, 3H).

### Example 101. Preparation of 3-(2-(hydroxymethyl)-1,3-oxathiolan-5-yl)-4-oxo-7-propyl-3H-pyrazolo[4,3-d][1,2,3]triazine-5(4H)-carbothioamide

Analogously to General Method C, the intermediate compound (I) (5-(4-butyryl-5,6-dioxo-5,6-dihydro-1,2,3-triazin-1(4H)-yl)-1,3-oxathiolan-2-yl)methyl acetate was prepared from 6-butyryl-1,2,3-triazine-4,5(3H,6H)-dione and (5-acetoxy-1,3-oxathiolan-2-yl)methyl acetate for next step. Analogously to General Method J, the intermediate compound (II) (5-(5-carbamothioyl-4-oxo-7-propyl-4,5-dihydro-3H-pyrazolo[4,3-d][1,2,3]triazin-3-yl)-1,3-oxathiolan-2-yl)methyl acetate was prepared from above intermediate compound (I) and hydrazinecarbothioamide for next step. Analogously to General Method E, the title compound was prepared from the intermediate compound (II) 3.98 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 8.56 (s, 2H), 5.22 (t, J=7.0 Hz, 1H), 4.03 (m, 2H), 3.97 (m, J=7.0 Hz, 1H), 3.65 (s, 1H), 2.83 (m, 2H), 2.44 (t, J=7.1 Hz, 2H), 1.65 (m, 2H), 0.90 (t, J=8.0 Hz, 3H).

### Example 102. Preparation of 2-(bis(isopropylamino)phosphoryl)-2-((5-(7-(ethoxycarbonyl)-5-hydroxy-4-oxo-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-(bis(isopropylamino) phosphoryl)-2-((3,4-diacetoxy-5-(4-(2-ethoxy-2-oxoacetyl)-5,6-dioxo-5,6-dihydro-1,2,3-triazin-1(4H)-yl) tetrahydrofuran-2-yl)methoxy)acetic acid was prepared from ethyl 2-(5,6-dioxo-1,4,5,6-tetrahydro-1,2,3-triazin-4-yl)-2-oxoacetate and hydroxyhydrazine for next step. Analogously to General Method J, the intermediate compound (II) 2-(bis(isopropylamino)phosphoryl)-2-((3,4-diacetoxy-5-(7-(ethoxycarbonyl)-5-hydroxy-4-oxo-4,5-dihydro-3H-pyrazolo[4,3-d][1,2,3]triazin-3-yl)tetrahydrofuran-2-yl)methoxy)acetic acid was prepared from above intermediate compound (I) and hydroxyhydrazine for next step. Analogously to General Method E, the title compound was prepared from the intermediate compound (II) 6.61 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.64 (m, J=7.0 Hz, 1H), 4.51 (t, J=7.0 Hz, 1H), 4.30 (m, J=8.0 Hz, 2H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.00 (s, 3H), 1.29 (t, J=8.0 Hz, 3H), 1.07 (d, J=6.8 Hz, 12H).

### Example 103. Preparation of 2-(bis(isopropylamino)phosphoryl)-2-((3,4-dihydroxy-5-(5-hydroxy-4-oxo-7-propyl-4,5-dihydro-3H-pyrazolo[4,3-d][1,2,3]triazin-3-yl)tetrahydrofuran-2-yl)methoxy)acetic acid

Analogously to General Method C, the intermediate compound (I) 2-(bis(isopropylamino) phosphoryl)-2-((3,4-diacetoxy-5-(4-butyryl-5,6-dioxo-5,6-dihydro-1,2,3-triazin-1(4H)-yl)tetrahydrofuran-2-yl)methoxy)acetic acid was prepared from 6-butyryl-1,2,3-triazine-4,5(3H,6H)-dione and hydroxyhydrazine for next step. Analogously to General Method J, the intermediate compound (II) 2-(bis(isopropylamino)phosphoryl)-2-((3,4-diacetoxy-5-(5-hydroxy-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydroxyhydrazine for next step. Analogously to General Method E, the title compound was prepared from the intermediate compound (II) 6.31 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): 611.00 (s, 1H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.44 (t, J=7.1 Hz, 2H), 2.00 (s, 3H), 1.65 (m, 2H), 1.07 (d, J=6.8 Hz, 12H), 0.90 (t, J=8.0 Hz, 3H).

### Example 104. Preparation of 2-((5-(7-amino-5-hydroxy-4-oxo-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid

Analogously to General Method C, the intermediate compound (I) 2-(bis(isopropylamino) phosphoryl)-2-((3,4-diacetoxy-5-(4-cyano-5,6-dioxo-5,6-dihydro-1,2,3-triazin-1(4H)-yl)tetrahydrofuran-2-yl)methoxy)acetic acid was prepared from 5,6-dioxo-1,4,5,6-tetrahydro-1,2,3-triazine-4-carbonitrile and hydroxyhydrazine for next step. Analogously to General Method J, the intermediate compound (II) 2-(bis(isopropylamino)phosphoryl)-2-((3,4-diacetoxy-5-(7-amino-5-hydroxy-4-oxo-4,5-dihydro-3H-pyrazolo[4,3-d][1,2,3]triazin-3-yl)tetrahydrofuran-2-yl)methoxy)acetic acid was prepared from above intermediate compound (I) and hydroxyhydrazine for next step. Analogously to General Method E, the title compound was prepared from the intermediate compound (II) 6.04 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.74 (s, 2H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.00 (s, 3H), 1.07 (d, J=6.8 Hz, 12H).

### Example 105. Preparation of 2-(bis(isopropylamino)phosphoryl)-2-((5-(7-(ethoxycarbonyl)-5-methyl-4-oxo-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-(bis(isopropylamino) phosphoryl)-2-((3,4-diacetoxy-5-(7-(ethoxycarbonyl)-5-methyl-4-oxo-4,5-dihydro-3H-pyrazolo[4,3-d][1,2,3]triazin-3-yl)tetrahy-drofuran-2-yl)methoxy)acetic acid was prepared from ethyl 5-methyl-4-oxo-4,5-dihydro-3H-pyrazolo[4,3-d][1,2,3]triazine-7-carboxylate and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.59 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.64 (m, J=7.0 Hz, 1H), 4.51 (t, J=7.0 Hz, 1H), 4.30 (m, J=8.0 Hz, 2H), 4.28 (t, J=7.0 Hz, 1H), 3.95 (s, 3H), 3.70 (d, J=11.5 Hz, 1H), 3.58 (s, 2H), 3.50 (m, 2H), 2.97 (m, J=6.8 Hz, 2H), 2.00 (s, 2H), 1.29 (t, J=8.0 Hz, 3H), 1.07 (d, J=6.8 Hz, 12H).

### Example 106. Preparation of 2-(bis(isopropylamino)phosphoryl)-2-((3,4-dihydroxy-5-(5-methyl-4-oxo-7-propyl-4,5-dihydro-3H-pyrazolo [4,3-d][1,2,3]triazin-3-yl)tetrahydrofuran-2-yl) methoxy)acetic acid

Analogously to General Method C, the intermediate compound (I) 2-(bis(isopropylamino) phosphoryl)-2-((3,4-diacetoxy-5-(5-methyl-4-oxo-7-propyl-4,5-dihydro-3H-pyrazolo[4,3-d][1,2,3]triazin-3-yl)tetrahydrofuran-2-yl)methoxy)acetic acid was prepared from 5-methyl-7-propyl-3H-pyrazolo[4,3-d][1,2,3]triazin-4(5H)-one and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.29 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.95 (s, 3H), 3.70 (d, J=11.5 Hz, 1H), 3.58 (s, 2H), 3.50 (m, 2H), 2.97 (m, J=6.8 Hz, 2H), 2.44 (t, J=7.1 Hz, 2H), 2.00 (s, 2H), 1.65 (m, 2H), 1.07 (d, J=6.8 Hz, 12H), 0.90 (t, J=8.0 Hz, 3H).

### Example 107. Preparation of 2-((5-(7-amino-5-methyl-4-oxo-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid

Analogously to General Method C, the intermediate compound (I) 2-(bis(isopropylamino) phosphoryl)-2-((3,4-diacetoxy-5-(7-amino-5-methyl-4-oxo-4,5-dihydro-3H-pyrazolo[4,3-d][1,2,3]triazin-3-yl)tetrahydrofuran-2-yl)methoxy)acetic acid was prepared from 7-amino-5-methyl-3H-pyrazolo[4,3-d][1,2,3]triazin-4(5H)-one and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.02 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.74 (s, 2H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.95 (s, 3H), 3.70 (d, J=11.5 Hz, 1H), 3.58 (s, 2H), 3.50 (m, 2H), 2.97 (m, J=6.8 Hz, 2H), 2.00 (s, 2H), 1.07 (d, J=6.8 Hz, 12H).

### Example 108. Preparation of 2-(bis(isopropylamino)phosphoryl)-2-((5-(7-cyano-5-methyl-4-oxo-4,5-dihydro-3H-pyrazolo[4,3-d][1,2,3]triazin-3-yl)-3,4-dihydroxytetrahydrofuran-2-yl) methoxy)acetic acid

Analogously to General Method C, the intermediate compound (I) 2-(bis(isopropylamino) phosphoryl)-2-((3,4-diacetoxy-5-(7-cyano-5-methyl-4-oxo-4,5-dihydro-3H-pyrazolo[4,3-d][1,2,3]triazin-3-yl)tetrahydrofuran-2-yl)methoxy)acetic acid was prepared from 5-methyl-4-oxo-4,5-dihydro-3H-pyrazolo[4,3-d][1,2,3]triazine-7-carbonitrile and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.12 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.95 (s, 3H), 3.70 (d, J=11.5 Hz, 1H), 3.58 (s, 2H), 3.50 (m, 2H), 2.97 (m, J=6.8 Hz, 2H), 2.00 (s, 2H), 1.07 (d, J=6.8 Hz, 12H).

### Example 109. Preparation of 2-(bis(isopropylamino)phosphoryl)-2-((5-(7-(ethoxycarbonyl)-5-(hydroxymethyl)-4-oxo-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-(bis(isopropylamino) phosphoryl)-2-((3,4-diacetoxy-5-(4-(2-ethoxy-2-oxoacetyl)-5,6-dioxo-5,6-dihydro-1,2,3-triazin-1(4H)-yl) tetrahydrofuran-2-yl)methoxy)acetic acid was prepared from ethyl 2-(5,6-dioxo-1,4,5,6-tetrahydro-1,2,3-triazin-4-yl)-2-oxo-acetate and 2-(bis(isopropylamino)phosphoryl)-2-((3,4,5-triacetoxytetrahydrofuran-2-yl)methoxy)acetic acid for next step. Analogously to General Method J, the intermediate compound (II) 2-(bis(isopropylamino)phosphoryl)-2-((3,4-diacetoxy-5-(7-(ethoxycarbonyl)-5-(hydroxymethyl)-4-oxo-4,5-dihydro-3H-pyrazolo[4,3-d][1,2,3]triazin-3-yl)tetrahydrofuran-2-yl)methoxy) acetic acid was prepared from above intermediate compound (I) and hydrazinylmethanol for next step. Analogously to General Method E, the title compound was prepared from the intermediate compound (II) 6.75 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromato-graphy to give the title compound. ¹H NMR (600 MHZ, CDCl₃-d₆): δ 11.00 (s, 1H), 5.97 (s, 2H), 5.93 (d, J=7.0 Hz, 1H), 4.64 (m, J=7.0 Hz, 1H), 4.51 (t, J=7.0 Hz, 1H), 4.30 (m, J=8.0 Hz, 2H), 4.28 (t, J=7.0 Hz, 1H), 3.70 (d, J=11.5 Hz, 1H), 3.65 (s, 1H), 3.58 (s, 2H), 3.50 (m, 2H), 2.97 (m, J=6.8 Hz, 2H), 2.00 (s, 2H), 1.29 (t, J=8.0 Hz, 3H), 1.07 (d, J=6.8 Hz, 12H).

### Example 110. Preparation of 2-(bis(isopropylamino)phosphoryl)-2-((3,4-dihydroxy-5-(5-(hydroxymethyl)-4-oxo-7-propyl-4,5-dihydro-3H-pyrazolo [4,3-d][1,2,3]triazin-3-yl) tetrahydrofuran-2-yl)methoxy)acetic acid

Analogously to General Method C, the intermediate compound (I) 2-(bis(isopropylamino) phosphoryl)-2-((3,4-diacetoxy-5-(4-butyryl-5,6-dioxo-5,6-dihydro-1,2,3-triazin-1(4H)-yl)tetrahydrofuran-2-yl)methoxy)acetic acid was prepared from 6-butyryl-1,2,3-triazine-4,5(3H,6H)-dione and 2-(bis(isopropylamino)phosphoryl)-2-((3,4,5-triacetoxytetrahydrofuran-2-yl)methoxy)acetic acid for next step. Analogously to General Method J, the intermediate compound (II) 2-(bis(isopropylamino) phosphoryl)-2-((3,4-diacetoxy-5-(5-(hydroxyl-methyl)-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydrazinylmethanol for next step. Analogously to General Method E, the title compound was prepared from the intermediate compound (II) 6.45 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.97 (s, 2H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.65 (s, 1H), 3.58 (s, 2H), 3.50 (m, 2H), 2.97 (m, J=6.8 Hz, 2H), 2.44 (t, J=7.1 Hz, 2H), 2.00 (s, 2H), 1.65 (m, 2H), 1.07 (d, J=6.8 Hz, 12H), 0.90 (t, J=8.0 Hz, 3H).

### Example 111. Preparation of 2-(bis(isopropylamino)phosphoryl)-2-((5-(5-cyclopropyl-4-oxo-7-propyl-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-(bis(isopropylamino) phosphoryl)-2-((3,4-diacetoxy-5-(5-cyclopropyl-4-oxo-7-propyl-4,5-dihydro-3H-pyrazolo[4,3-d][1,2,3]triazin-3-yl)tetrahy-drofuran-2-yl)methoxy)acetic acid was prepared from 5-cyclopropyl-7-propyl-3H-pyrazolo[4,3-d][1,2,3]triazin-4(5H)-one and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.55 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.44 (t, J=7.1 Hz, 2H), 2.42 (m, J=7.0 Hz, 1H), 2.00 (s, 2H), 1.65 (m, 2H), 1.07 (d, J=6.8 Hz, 12H), 0.90 (t, J=8.0 Hz, 3H), 0.66 (m, 4H).

### Example 112. Preparation of 2-((5-(5-(2-amino-2-oxoethyl)-4-oxo-7-propyl-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid

Analogously to General Method C, the intermediate compound (I) 2-(bis(isopropylamino) phosphoryl)-2-((3,4-diacetoxy-5-(4-butyryl-5,6-dioxo-5,6-dihydro-1,2,3-triazin-1(4H)-yl)tetrahydrofuran-2-yl)methoxy)acetic acid was prepared from 6-butyryl-1,2,3-triazine-4,5(3H,6H)-dione and 2-(bis(isopropylamino)phosphoryl)-2-((3,4,5-triacetoxytetrahydrofuran-2-yl)methoxy)acetic acid for next step. Analogously to General Method J, the intermediate compound (II) 2-(bis(isopropylamino) phosphoryl)-2-((3,4-diacetoxy-5-(5-(2-amino-2-oxoethyl)-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and 2-hydrazinylacetamide for next step. Analogously to General Method E, the title compound was prepared from the intermediate compound (II) 6.45 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.16 (s, 2H), 5.93 (d, J=7.0 Hz, 1H), 5.60 (s, 2H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.44 (t, J=7.1 Hz, 2H), 2.00 (s, 2H), 1.65 (m, 2H), 1.07 (d, J=6.8 Hz, 12H), 0.90 (t, J=8.0 Hz, 3H).

### Example 113. Preparation of 2-((5-(7-amino-5-(2-amino-2-oxoethyl)-4-oxo-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid

Analogously to General Method C, the intermediate compound (I) 2-(bis(isopropylamino) phosphoryl)-2-((3,4-diacetoxy-5-(4-cyano-5,6-dioxo-5,6-dihydro-1,2,3-triazin-1(4H)-yl)tetrahydrofuran-2-yl)methoxy)acetic acid was prepared from 6-dioxo-1,4,5,6-tetrahydro-1,2,3-triazine-4-carbonitrile an d2-(bis(isopropyl-amino)phosphoryl)-2-((3,4,5-triacetoxytetrahydrofuran-2-yl)methoxy)acetic acid for next step. Analogously to General Method J, the intermediate compound (II) 2-(bis(isopropylamino)phosphoryl)-2-((3,4-diacetoxy-5-(7-amino-5-(2-amino-2-oxoethyl)-4-oxo-4,5-dihydro-3H-pyrazolo[4,3-d][1,2,3]triazin-3-yl)tetrahydrofuran-2-yl)methoxy)acetic acid was prepared from above intermediate compound (I) and 2-hydrazinylacetamide for next step. Analogously to General Method E, the title compound was prepared from the intermediate compound (II) 6.45 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.74 (s, 2H), 7.16 (s, 2H), 5.93 (d, J=7.0 Hz, 1H), 5.60 (s, 2H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.00 (s, 2H), 1.07 (d, J=6.8 Hz, 12H).

### Example 114. Preparation of 2-(bis(isopropylamino)phosphoryl)-2-((5-(5-carbamoyl-4-oxo-7-propyl-4,5-dihydro-3H-pyrazolo[4,3-d][1,2,3]triazin-3-yl)-3,4-dihydroxytetrahydrofuran-2-yl) methoxy)acetic acid

Analogously to General Method C, the intermediate compound (I) 2-(bis(isopropylamino) phosphoryl)-2-((3,4-diacetoxy-5-(4-butyryl-5,6-dioxo-5,6-dihydro-1,2,3-triazin-1(4H)-yl)tetrahydrofuran-2-yl)methoxy)acetic acid was prepared from 6-butyryl-1,2,3-triazine-4,5(3H,6H)-dione and 2-(bis(isopropylamino)phosphoryl)-2-((3,4,5-triacetoxytetrahydrofuran-2-yl)methoxy)acetic acid for next step. Analogously to General Method J, the intermediate compound (II) 2-(bis(isopropylamino) phosphoryl)-2-((3,4-diacetoxy-5-(5-carbamoyl-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydrazinecar-boxamide for next step. Analogously to General Method E, the title compound was prepared from the intermediate compound (II) 6.58 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.68 (s, 2H), 5.93 (d, J=7.0 Hz, 1H), 4.64 (m, J=7.0 Hz, 1H), 4.51 (t, J=7.0 Hz, 1H), 4.28 (t, J=7.0 Hz, 1H), 3.70 (d, J=11.5 Hz, 1H), 3.58 (s, 2H), 3.50 (m, 2H), 2.97 (m, J=6.8 Hz, 2H), 2.44 (t, J=7.1 Hz, 2H), 2.00 (s, 2H), 1.65 (m, 2H), 1.07 (d, J=6.8 Hz, 12H), 0.90 (t, J=8.0 Hz, 3H).

### Example 115. Preparation of 2-(bis(isopropylamino)phosphoryl)-2-(((5-(5-carbamoyl-7-(3-(dimethylamino)propanamido)-4-oxo-4,5-dihydro-3H-pyrazolo [4,3-d][1,2,3]triazin-3-yl)-3,4-dihydroxytetrahydrofuran-2-yl)methyl)amino)acetic acid

Analogously to General Method C, the intermediate compound (I) 2-(bis(isopropylamino) phosphoryl)-2-((3,4-diacetoxy-5-(4-cyano-5,6-dioxo-5,6-dihydro-1,2,3-triazin-1(4H)-yl)tetrahydrofuran-2-yl)methoxy)acetic acid was prepared from 5,6-dioxo-1,4,5,6-tetrahydro-1,2,3-triazine-4-carbonitrile and 2-(acetoxymethoxy)-2-(bis(isopropylamino)phosphoryl)acetic acid for next step. Analogously to General Method J, the interme-diate compound (II) 2-(bis(isopropylamino)phosphoryl)-2-((3,4-diacetoxy-5-(7-amino-5-carbamoyl-4-oxo-4,5-dihydro-3H-pyrazolo[4,3-d][1,2,3]triazin-3-yl) tetrahydrofuran-2-yl)methoxy)acetic acid was prepared from above intermediate compound (I) and hydrazinecarboxamide for next step. Analogously to General Method K, the intermediate compound (III) 2-(bis(isopropylamino)phosphoryl)-2-((3,4-diacetoxy-5-(5-carbamoyl-7-(3-(dimethylamino)propanamido)-4-oxo-4,5-dihydro-3H-pyrazolo[4,3-d][1,2,3]triazin-3-yl)tetrahydrofuran-2-yl)methoxy)acetic acid was prepared from above intermediate compound (II) and 3-(dimethylamino)propanoyl chloride for next step. Analogously to General Method E, the title compound was prepared from the intermediate compound (III) 7.30 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 11.00 (s, 1H), 9.15 (s, 1H), 7.68 (s, 2H), 5.93 (d, J=7.0 Hz, 1H), 4.51 (t, J=7.0 Hz, 1H), 4.28 (t, J=7.0 Hz, 1H), 4.06 (m, J=7.0 Hz, 1H), 3.65 (t, J=7.1 Hz, 2H), 3.58 (s, 2H), 3.50 (d, J=11.5 Hz, 1H), 2.97 (m, J=6.8 Hz, 2H), 2.84 (s, 6H), 2.68 (m, 2H), 2.50 (t, J=7.1 Hz, 2H), 2.00 (s, 3H), 1.07 (d, J=6.8 Hz, 12H).

### Example 116. Preparation of 2-((cyanomethoxy)(isopropylamino)phosphino)-2-((5-(7-(ethoxycarbonyl)-5-hydroxy-4-oxo-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-((cyanomethoxy) (isopropylamino)phosphino)-2-((3,4-diacetoxy-5-(4-(2-ethoxy-2-oxoacetyl)-5,6-dioxo-5,6-dihydro-1,2,3-triazin-1(4H)-yl)tetrahydrofuran-2-yl)methoxy)acetic acid was prepared from ethyl 2-(5,6-dioxo-1,4,5,6-tetrahydro-1,2,3-triazin-4-yl)-2-oxoacetate and 2-(bis(isopropylamino)phosphoryl)-2-((3,4,5-triacetoxytetrahydrofuran-2-yl)methoxy)acetic acid for next step. Analogously to General Method J, the intermediate compound (II) 2-((cyanomethoxy)(isopropylamino)phosphino)-2-((3,4-diacetoxy-5-(7-(ethoxycarbonyl)-5-hydroxy-4-oxo-4,5-dihydro-3H-pyrazolo[4,3-d][1,2,3]triazin-3-yl)tetrahydrofuran-2-yl)methoxy)acetic acid was prepared from above intermediate compound (I) and hydroxyhydrazine for next step. Analogously to General Method E, the title compound was prepared from the intermediate compound (II) 6.58 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.6 (m, J=8.0 Hz, 2H), 4.64 (t, J=7.0 Hz, 1H), 4.51 (t, J=7.0 Hz, 1H), 4.30 (m, J=8.0 Hz, 2H), 4.28 (m, J=7.0 Hz, 1H), 3.70 (s, 1H), 3.58 (s, 2H), 3.50 (m, 2H), 2.97 (m, J=6.8 Hz, 1H), 2.00 (s, 2H), 1.29 (t, J=8.0 Hz, 3H), 1.07 (d, J=6.8 Hz, 6H).

### Example 117. Preparation of 2-((cyanomethoxy)(isopropylamino)phosphino)-2-((3,4-dihydroxy-5-(5-hydroxy-4-oxo-7-propyl-4,5-dihydro-3H-pyrazolo [4,3-d][1,2,3]triazin-3-yl) tetrahydrofuran-2-yl)methoxy)acetic acid

Analogously to General Method C, the intermediate compound (I) 2-((cyanomethoxy) (isopropylamino)phosphino)-2-((3,4-diacetoxy-5-(4-butyryl-5,6-dioxo-5,6-dihydro-1,2,3-triazin-1(4H)-yl) tetrahydrofuran-2-yl)methoxy)acetic acid was prepared from 6-butyryl-1,2,3-triazine-4,5(3H,6H)-dion and 2-((cyano-methoxy)(isopropylamino)phosphino)-2-((3,4,5-triacetoxytetrahydrofuran-2-yl)methoxy) acetic acid for next step. Analogously to General Method J, the intermediate compound (II) 2-((cyanomethoxy)(isopropyl-amino)phosphino)-2-((3,4-diacetoxy-5-(5-hydroxy-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydroxyhydrazine for next step. Analogously to General Method E, the title compound was prepared from the intermediate compound (II) 6.13 g (10 mmol) and the solution of methanol/NaOCH₃ 20 mL (1 mol/L). The mixture was reacted For 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.44 (t, J=7.1 Hz, 2H), 2.00 (s, 2H), 1.65 (m, 2H), 1.07 (d, J=6.8 Hz, 6H), 0.90 (t, J=8.0 Hz, 3H).

### Example 118. Preparation of 2-((5-(7-cyano-5-hydroxy-4-oxo-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid

Analogously to General Method C, the intermediate compound (I) 2-((cyanomethoxy) (isopropylamino)phosphino)-2-((3,4-diacetoxy-5-(4-(cyanocarbonyl)-5,6-dioxo-5,6-dihydro-1,2,3-triazin-1(4H)-yl)tetrahy-drofuran-2-yl)methoxy)acetic acid was prepared from 5,6-dioxo-1,4,5,6-tetrahydro-1,2,3-triazine-4-carbonyl cyanide and 2-((cyanomethoxy)(isopropylamino)phosphino)-2-((3,4,5-triacetoxytetrahydrofuran-2-yl)methoxy)acetic acid for next step. Analogously to General Method J, the intermediate compound (II) 2-((cyanomethoxy)(isopropylamino)phosphino)-2-((3,4-diacetoxy-5-(7-cyano-5-hydroxy-4-oxo-4,5-dihydro-3H-pyrazolo[4,3-d][1,2,3]triazin-3-yl)tetrahydrofuran-2-yl) methoxy)acetic acid was prepared from above intermediate compound (I) and hydroxyhydrazine for next step. Analogously to General Method E, the title compound was prepared from the intermediate compound (II) 6.13 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.00 (s, 2H), 1.07 (d, J=6.8 Hz, 6H).

### Example 119 Preparation of 2-((cyanomethoxy)(isopropylamino)phosphino)-2-((5-(7-(ethoxycarbonyl)-5-methyl-4-oxo-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-((cyanomethoxy) (isopropylamino)phosphino)-2-((3,4-diacetoxy-5-(7-(ethoxycarbonyl)-5-methyl-4-oxo-4,5-dihydro-3H-pyrazolo[4,3-d][1,2,3]triazin-3-yl)tetrahydrofuran-2-yl)methoxy)acetic acid was prepared from ethyl 5-methyl-4-oxo-4,5-dihydro-3H-pyrazolo[4,3-d][1,2,3]triazine-7-carboxylate and 2-(bis(isopropylamino) phosphoryl)-2-((3,4,5-triacetoxytetrahydrofuran-2-yl)methoxy)acetic acid for next step. Analogously to General Method E, the title compound was prepared from the intermediate compound (I) 6.41 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.30 (m, J=8.0 Hz, 2H), 4.28 (m, J=7.0 Hz, 1H), 3.95 (s, 3H), 3.70 (s, 1H), 3.58 (s, 2H), 3.50 (m, 2H), 2.97 (m, J=6.8 Hz, 1H), 2.00 (s, 1H), 1.29 (t, J=8.0 Hz, 3H), 1.07 (d, J=6.8 Hz, 6H).

### Example 120. Preparation of 2-((cyanomethoxy)(isopropylamino)phosphino)-2-((3,4-dihydroxy-5-(5-methyl-4-oxo-7-propyl-4,5-dihydro-3H-pyrazolo[4,3-d][1,2,3]triazin-3-yl) tetrahydrofuran-2-yl)methoxy)acetic acid

Analogously to General Method C, the intermediate compound (I) 2-((cyanomethoxy) (isopropylamino)phosphino)-2-((3,4-diacetoxy-5-(5-methyl-4-oxo-7-propyl-4,5-dihydro-3H-pyrazolo[4,3-d][1,2,3]triazin-3-yl)tetrahydrofuran-2-yl)methoxy)acetic acid was prepared from 5-methyl-7-propyl-3H-pyrazolo[4,3-d][1,2,3]triazin-4(5H)-one and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.11 g (10 mmol) and the solution of methanol/NaOCH3 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.95 (s, 3H), 3.70 (s, 1H), 3.58 (s, 2H), 3.50 (m, 2H), 2.97 (m, J=6.8 Hz, 1H), 2.44 (t, J=7.1 Hz, 2H), 2.00 (s, 1H), 1.65 (m, 2H), 1.07 (d, J=6.8 Hz, 6H), 0.90 (t, J=8.0 Hz, 3H).

### Example 121. Preparation of 2-((5-(5-(2-amino-2-oxoethyl)-7-(ethoxycarbonyl)-4-oxo-4,5-dihydro-3H-pyrazolo[4,3-d][1,2,3]triazin-3-yl)-3,4-dihydroxytetrahydrofuran-2-yl)methoxy)-2-((cyanomethoxy)(isopropylamino)phosphino)acetic acid

Analogously to General Method C, the intermediate compound (I) 2-((cyanomethoxy) (isopropylamino)phosphino)-2-((3,4-diacetoxy-5-(4-(2-ethoxy-2-oxoacetyl)-5,6-dioxo-5,6-dihydro-1,2,3-triazin-1(4H)-yl)tetrahydrofuran-2-yl)methoxy)acetic acid was prepared from ethyl 2-(5,6-dioxo-1,4,5,6-tetrahydro-1,2,3-triazin-4-yl)-2-oxoacetate and 2-((cyanomethoxy)(isopropylamino)phosphino)-2-((3,4,5-triacetoxytetrahy-drofuran-2-yl)methoxy)acetic acid for next step. Analogously to General Method J, the intermediate compound (II) 2-((cyanomethoxy)(isopropylamino)phosphino)-2-((3,4-diacetoxy-5-(5-(2-amino-2-oxoethyl)-7-(ethoxycarbonyl)-4-oxo-4,5-dihydro-3H-pyrazolo[4,3-d][1,2,3]triazin-3-yl)tetrahydrofuran-2-yl)methoxy)acetic acid was prepared from above intermediate compound (I) and 2-hydrazinylacetamide for next step. Analogously to General Method E, the title compound was prepared from the intermediate compound (II) 6.84 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.16 (s, 2H), 5.93 (d, J=7.0 Hz, 1H), 5.60 (s, 2H), 4.65 (m, J=8.0 Hz, 2H), 4.64 (t, J=7.0 Hz, 1H), 4.51 (t, J=7.0 Hz, 1H), 4.30 (m, J=8.0 Hz, 2H), 4.28 (m, J=7.0 Hz, 1H), 3.70 (s, 1H), 3.58 (s, 2H), 3.50 (m, 2H), 2.97 (m, J=6.8 Hz, 1H), 2.00 (s, 1H), 1.29 (t, J=8.0 Hz, 3H), 1.07 (d, J=6.8 Hz, 6H).

### Example 122. Preparation of 2-((cyanomethoxy)(isopropylamino)phosphino)-2-((5-(5-cyclopropyl-4-oxo-7-propyl-4,5-dihydro-3H-pyrazolo[4,3-d][1,2,3]triazin-3-yl)-3,4-dihydroxytetrahydrofuran-2-yl)methoxy)acetic acid

Analogously to General Method C, the intermediate compound (I) 2-((cyanomethoxy) (isopropylamino)phosphino)-2-((3,4-diacetoxy-5-(5-cyclopropyl-4-oxo-7-propyl-4,5-dihydro-3H-pyrazolo[4,3-d][1,2,3]triazin-3-yl)tetrahydrofuran-2-yl)methoxy)acetic acid was prepared from 5-cyclopropyl-7-propyl-3H-pyrazolo[4,3-d][1,2,3]triazin-4(5H)-one and 2-(bis(isopropylamino)phosphoryl)-2-((3,4,5-triacetoxytetrahydrofuran-2-yl)methoxy)acetic acid for next step. Analogously to General Method E, the title compound was prepared from the intermediate compound (I) 6.37 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.44 (t, J=7.1 Hz, 2H), 2.42 (m, J=7.0 Hz, 1H), 2.00 (s, 1H), 1.65 (m, 2H), 1.07 (d, J=6.8 Hz, 6H), 0.90 (t, J=8.0 Hz, 3H), 0.66 (m, 4H).

### Example 123. Preparation of 2-((5-(5-carbamoyl-7-cyano-4-oxo-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid

Analogously to General Method C, the intermediate compound (I) 2-((cyanomethoxy) (isopropylamino)phosphino)-2-((3,4-diacetoxy-5-(4-(cyanocarbonyl)-5,6-dioxo-5,6-dihydro-1,2,3-triazin-1(4H)-yl)tetrahydro-furan-2-yl)methoxy)acetic acid was prepared from 5,6-dioxo-1,4,5,6-tetrahydro-1,2,3-triazine-4-carbonyl cyanide and 2-((cyanomethoxy)(isopropylamino)phosphino)-2-((3,4,5-triacetoxytetrahydrofuran-2-yl)methoxy)acetic acid for next step. Analogously to General Method J, the intermediate compound (II) 2-((cyanomethoxy)(isopropylamino)phosphino)-2-((3,4-diacetoxy-5-(5-carbamoyl-7-cyano-4-oxo-4,5-dihydro-3H-pyrazolo[4,3-d][1,2,3]triazin-3-yl)tetrahydrofuran-2-yl) methoxy)acetic acid was prepared from above intermediate compound (I) and hydrazinecarboxamide for next step. Analogously to General Method E, the title compound was prepared from the intermediate compound (II) 6.23 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.68 (s, 2H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.00 (s, 1H), 1.07 (d, J=6.8 Hz, 6H).

### Example 124. Preparation of 2-((5-(5-carbamimidoyl-4-oxo-7-propyl-4,5-dihydro-3H-pyrazolo[4,3-d][1,2,3]triazin-3-yl)-3,4-dihydroxytetrahydrofuran-2-yl)methoxy)-2-((cyanomethoxy)(isopropylamino)phosphino)acetic acid

Analogously to General Method C, the intermediate compound (I) 2-((cyanomethoxy) (isopropylamino)phosphino)-2-((3,4-diacetoxy-5-(4-butyryl-5,6-dioxo-5,6-dihydro-1,2,3-triazin-1(4H)-yl) tetrahydrofuran-2-yl)methoxy)acetic acid was prepared from 6-butyryl-1,2,3-triazine-4,5(3H,6H)-dione and 2-((cyano-methoxy)(isopropylamino)phosphino)-2-((3,4,5-triacetoxytetrahydrofuran-2-yl)methoxy) acetic acid for next step. Analogously to General Method J, the intermediate compound (II) 2-((cyanomethoxy)(isopropyl-amino)phosphino)-2-((3,4-diacetoxy-5-(5-carbamimidoyl-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydrazinecarboximidamide for next step. Analogously to General Method E, the title compound was prepared from the intermediate compound (II) 6.39 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound.

¹H NMR (600 MHZ, CDCl₃-d₆): δ 11.00 (s, 1H), 8.56 (s, 2H), 8.03 (s, 1H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.44 (t, J=7.1 Hz, 2H), 2.00 (s, 1H), 1.65 (m, 2H), 1.07 (d, J=6.8 Hz, 6H), 0.90 (t, J=8.0 Hz, 3H).

### Example 125. Preparation of 2-((5-(5-carbamothioyl-4-oxo-7-propyl-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid

Analogously to General Method C, the intermediate compound (I) 2-((cyanomethoxy) (isopropylamino)phosphino)-2-((3,4-diacetoxy-5-(4-butyryl-5,6-dioxo-5,6-dihydro-1,2,3-triazin-1(4H)-yl) tetrahydrofuran-2-yl)methoxy)acetic acid was prepared from 6-butyryl-1,2,3-triazine-4,5(3H,6H)-dione and 2-((cyano-methoxy)(isopropylamino)phosphino)-2-((3,4,5-triacetoxytetrahydrofuran-2-yl)methoxy) acetic acid for next step. Analogously to General Method J, the intermediate compound (II) 2-((cyanomethoxy)(isopropyl-amino)phosphino)-2-((3,4-diacetoxy-5-(5-carbamothioyl-4-oxo-7-propyl-4,5-dihydro-3H-pyrazolo[4,3-d][1,2,3]triazin-3-yl)tetrahydrofuran-2-yl)methoxy)acetic acid was prepared from above intermediate compound (I) and hydrazinecarboximidamide for next step. Analogously to General Method E, the title compound was prepared from the intermediate compound (II) 6.56 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 8.56 (s, 2H), 5.93 (d, J=7.0 Hz, 1H), 4.65 (m, J=8.0 Hz, 2H), 4.64 (t, J=7.0 Hz, 1H), 4.51 (t, J=7.0 Hz, 1H), 4.28 (m, J=7.0 Hz, 1H), 3.70 (s, 1H), 3.58 (s, 2H), 3.50 (m, 2H), 2.97 (m, J=6.8 Hz, 1H), 2.44 (t, J=7.1 Hz, 2H), 2.00 (s, 1H), 1.65 (m, 2H), 1.07 (d, J=6.8 Hz, 6H), 0.90 (t, J=8.0 Hz, 3H).

### Example 126. Preparation of 3-(((1,3-dihydroxypropan-2-yl)oxy)methyl)-5-methyl-7-propyl-3H-pyrazolo[4,3-d][1,2,3]triazin-4(5H)-one

Analogously to General Method C, the intermediate compound (I) 2-((5-methyl-4-oxo-7-propyl-4,5-dihydro-3H-pyrazolo[4,3-d][1,2,3]triazin-3-yl)methoxy)propane-1,3-diyl diacetate was prepared from 5-methyl-7-propyl-3H-pyrazolo[4,3-d][1,2,3]triazin-4(5H)-one and 2-(acetoxymethoxy)propane-1,3-diyl diacetate for next step. Analogously to General Method E, the title compound was prepared from the intermediate compound (I) 3.81 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.04 (s, 2H), 3.95 (s, 3H), 3.65 (s, 2H), 3.48 (d, J=7.0 Hz, 4H), 3.01 (m, J=7.0 Hz, 1H), 2.44 (t, J=7.1 Hz, 2H), 1.65 (m, 2H), 0.90 (t, J=8.0 Hz, 3H).

### Example 127. Preparation of 2-(diethoxyphosphoryl)-2-((3-(((1,3-dihydroxypropan-2-yl) oxy)methyl)-5-(hydroxymethyl)-4-oxo-4,5-dihydro-3H-pyrazolo[4,3-d][1,2,3]triazin-7-yl)amino) acetic acid

Analogously to General Method C, the intermediate compound (I) 2-((4-cyano-5,6-dioxo-5,6-dihydro-1,2,3-triazin-1(4H)-yl)methoxy)propane-1,3-diyl diacetate was prepared from 5,6-dioxo-1,4,5,6-tetrahydro-1,2,3-triazine-4-carbonitrile and 2-(acetoxymethoxy)propane-1,3-diyl diacetate acid for next step. Analogously to General Method J, the intermediate compound (II) 2-((7-amino-5-(hydroxymethyl)-4-oxo-4,5-dihydro-3H-pyrazolo[4,3-d][1,2,3]triazin-3-yl)methoxy)propane-1,3-diyl diacetate was prepared from above intermediate compound (I) and hydrazinylmethanol for next step. Analogously to General Method K, the intermediate compound (III) 2-((3-(((1,3-diacetoxypropan-2-yl)oxy)methyl)-5-(hydroxymethyl)-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 7.30 g (10 mmol) and the solution of methanol/NaOCH₃ 20 mL (1 mol/L). The mixture was reacted for 5 h and added acetic acid to pH˜7. The reaction solution was extracted by EtOAc.

The crude was separated by silica gel column chromatography to give the title compound.

¹H NMR (600 MHZ, CDCl₃-d₆): δ 11.00 (s, 1H), 5.97 (s, 2H), 5.04 (s, 2H), 4.07 (m, 4H), 4.00 (s, 1H), 3.65 (s, 3H), 3.50 (d, J=11.5 Hz, 1H), 3.48 (d, J=7.0 Hz, 4H), 3.01 (m, J=7.0 Hz, 1H), 1.29 (t, 6H).

### Example 128. Preparation of 2-((5-(2-amino-2-oxoethyl)-3-(((1,3-dihydroxypropan-2-yl) oxy)methyl)-4-oxo-4,5-dihydro-3H-pyrazolo[4,3-d][1,2,3]triazin-7-yl)amino)-2-(diethoxyphosphoryl)acetic acid

Analogously to General Method C, the intermediate compound (I) 2-((4-cyano-5,6-dioxo-5,6-dihydro-1,2,3-triazin-1(4H)-yl)methoxy)propane-1,3-diyl diacetate was prepared from 5,6-dioxo-1,4,5,6-tetrahydro-1,2,3-triazine-4-carbonitrile and 2-(acetoxymethoxy)propane-1,3-diyl diacetate for next step. Analogously to General Method J, the intermediate compound (II) 2-((7-amino-5-(2-amino-2-oxoethyl)-4-oxo-4,5-dihydro-3H-pyrazolo[4,3-d][1,2,3]triazin-3-yl)methoxy)propane-1,3-diyl diacetate was prepared from above intermediate compound (I) and 2-hydrazinylacetamide for next step. Analogously to General Method K, the intermediate compound (III) 2-((5-(2-amino-2-oxoethyl)-3-(((1,3-diacetoxypropan-2-yl)oxy)methyl)-4-oxo-4,5-dihydro-3H-pyrazolo[4,3-d][1,2,3]triazin-7-yl)amino)-2-(diethoxyphosphoryl)acetic acid was prepared from above intermediate compound (II) and 2-chloro-2-(diethoxyphosphoryl)acetic acid for next step. Analogously to General Method E, the title compound was prepared from the intermediate compound (III) 5.91 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 7.16 (s, 2H), 5.60 (s, 2H), 5.04 (s, 2H), 4.07 (m, 4H), 4.00 (s, 1H), 3.65 (s, 2H), 3.50 (d, J=11.5 Hz, 1H), 3.48 (d, J=7.0 Hz, 4H), 3.01 (m, J=7.0 Hz, 1H), 1.29 (t, 6H).

### Example 129. Preparation of N-(5-(2-amino-2-oxoethyl)-3-(((1,3-dihydroxypropan-2-yl) oxy)methyl)-4-oxo-4,5-dihydro-3H-pyrazolo[4,3-d][1,2,3]triazin-7-yl)-3-(dimethylamino) propanamide

Analogously to General Method C, the intermediate compound (I) 2-((4-cyano-5,6-dioxo-5,6-dihydro-1,2,3-triazin-1(4H)-yl)methoxy)propane-1,3-diyl diacetate was prepared from 5,6-dioxo-1,4,5,6-tetrahydro-1,2,3-triazine-4-carbonitrile and 2-(acetylperoxy)propane-1,3-diyl diacetate for next step. Analogously to General Method J, the intermediate compound (II) 2-((7-amino-5-(2-amino-2-oxoethyl)-4-oxo-4,5-dihydro-3H-pyrazolo[4,3-d][1,2,3]triazin-3-yl)methoxy)propane-1,3-diyl diacetate was prepared from the intermediate compound (I) and 2-hydrazinylacetamide for next step. Analogously to General Method K, the intermediate compound (III) 2-((5-(2-amino-2-oxoethyl)-7-(3-(dimethylamino)propanamido)-4-oxo-4,5-dihydro-3H-pyrazolo[4,3-d][1,2,3]triazin-3-yl)methoxy) propane-1,3-diyl diacetate was prepared from above intermediate compound (II) and 3-(dimethylamino)propanoylchloride for next step. Analogously to General Method E, the title compound was prepared from the intermediate compound (III) 4.96 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 9.15 (s, 1H), 7.16 (s, 2H), 5.60 (s, 2H), 5.04 (s, 2H), 3.65 (t, J=7.1 Hz, 2H), 3.60 (s, 2H), 3.48 (d, J=7.0 Hz, 4H), 3.01 (m, J=7.0 Hz, 1H), 2.84 (s, 6H), 2.50 (t, J=7.1 Hz, 2H).

### Example 130. Preparation of 3-(((1,3-dihydroxypropan-2-yl)oxy)methyl)-4-oxo-7-propyl-3H-pyrazolo[4,3-d][1,2,3]triazine-5(4H)-carboximidamide

Analogously to General Method C, the intermediate compound (I) 2-((4-butyryl-5,6-dioxo-5,6-dihydro-1,2,3-triazin-1(4H)-yl)methoxy)propane-1,3-diyl diacetate was prepared from 6-butyryl-1,2,3-triazine-4,5(3H, 6H)-dione and 2-(acetoxymethoxy)propane-1,3-diyl diacetate for next step. Analogously to General Method J, the intermediate compound (II) 2-((5-carbamimidoyl-4-oxo-7-propyl-4,5-dihydro-3H-pyrazolo[4,3-d][1,2,3]triazin-3-yl)methoxy)propane-1,3-diyl diacetate was prepared from above intermediate compound (I) and hydrazinecarboximidamide for next step. Analogously to General Method E, the title compound was prepared from the intermediate compound (II) 4.09 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 8.56 (s, 2H), 8.03 (s, 1H), 5.04 (s, 2H), 3.65 (s, 2H), 3.48 (d, J=7.0 Hz, 4H), 3.01 (m, J=7.0 Hz, 1H), 2.44 (t, J=7.1 Hz, 2H), 1.65 (m, 2H), 0.90 (t, J=8.0 Hz, 3H).

### Example 131. Preparation of isopropyl 2-((((5-(5-carbamimidoyl-4-oxo-7-propyl-4,5-dihydro-3H-pyrazolo[4,3-d][1,2,3]triazin-3-yl)tetrahydrofuran-2-yl)methoxy)(phenoxy) phosphorothioyl)amino)propanoate

Analogously to General Method C, the intermediate compound (I) isopropyl 2-((((5-(4-butyryl-5,6-dioxo-5,6-dihydro-1,2,3-triazin-1(4H)-yl)tetrahydrofuran-2-yl)methoxy)(phenoxy)phosphorothioyl) amino)propanoate was prepared from 6-butyryl-1,2,3-triazine-4,5(3H,6H)-dione and isopropyl 2-((((5-acetoxytetrahydro-furan-2-yl)methoxy)(phenoxy)phosphorothioyl)amino)propanoate for next step. Analogously to General Method J, the title compound was prepared from above intermediate compound (I) 5.68 g (10 mmol) and hydrazinecarboximidamide 0.74 g (10 mmol). ¹H NMR (600 MHZ, CDCl₃-d₆): δ 8.56 (s, 2H), 8.03 (s, 1H), 7.28 (t, 2H), 7.21 (t, 2H), 7.18 (m, 1H), 5.85 (t, J=7.0 Hz, 1H), 4.93 (m, J=6.8 Hz, 1H), 3.83 (m, J=7.0 Hz, 1H), 3.65 (m, 2H), 3.63 (m, J=6.8 Hz, 1H), 2.44 (t, J=7.1 Hz, 2H), 2.07 (m, 2H), 2.00 (s, 1H), 1.81 (m, 2H), 1.65 (m, 2H), 1.32 (d, J=6.8 Hz, 6H), 1.28 (d, J=6.8 Hz, 3H), 0.90 (t, J=8.0 Hz, 3H).

### Example 132. Preparation of ethyl ethyl 3-(3,4-dihydroxy-5-(((hydroxy((hydroxy (phosphonooxy)phosphoryl)oxy)phosphoryl)oxy)methyl)tetrahydrofuran-2-yl)-5-hydroxy-4-oxo-4,5-dihydro-3H-pyrazolo [4,3-d][1,2,3]triazine-7-carboxylate

Analogously to General Method C, the intermediate compound (I) 2-(4-(2-ethoxy-2-oxoacetyl)-5,6-dioxo-5,6-dihydro-1,2,3-triazin-1(4H)-yl)-5-(((hydroxy((hydroxy(phosphonooxy)phosphoryl)oxy) phosphoryl)oxy)methyl)tetrahydrofuran-3,4-diyl diacetate was prepared from ethyl 2-(5,6-dioxo-1,4,5,6-tetrahydro-1,2,3-triazin-4-yl)-2-oxoacetate and 5-(((hydroxy((hydroxy(phosphonooxy)phosphoryl)oxy) phosphoryl)oxy)methyl)tetrahydrofuran-2,3,4-triyl triacetate for next step. Analogously to General Method J, the intermediate compound (II) 2-(7-(ethoxycarbonyl)-5-hydroxy-4-oxo-4,5-dihydro-3H-pyrazolo[4,3-d][1,2,3]triazin-3-yl)-5-(((hydroxy((hydroxy(phosphonooxy)phosphoryl)oxy)phosphoryl) oxy)methyl)tetrahydrofuran-3,4-diyl diacetate was prepared from above intermediate compound (I) and hydroxyhydrazine for next step. Analogously to General Method E, the title compound was prepared from the intermediate compound (II) 4.09 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.30 (m, J=8.0 Hz, 2H), 4.28 (m, J=7.0 Hz, 1H), 4.15 (m, 2H), 3.58 (s, 2H), 2.00 (s, 1H), 1.29 (t, J=8.0 Hz, 3H).

### Example 133. Preparation of (3,4-dihydroxy-5-(5-hydroxy-4-oxo-7-propyl-4,5-dihydro-3H-pyrazolo[4,3-d][1,2,3]triazin-3-yl)tetrahydrofuran-2-yl)methyl tetrahydrogen triphosphate

Analogously to General Method C, the intermediate compound (I) 2-(4-butyryl-5,6-dioxo-5,6-dihydro-1,2,3-triazin-1(4H)-yl)-5-(((hydroxy((hydroxy(phosphonooxy)phosphoryl)oxy)phosphoryl)oxy) methyl)tetrahydro-furan-3,4-diyl diacetate was prepared from 6-butyryl-1,2,3-triazine-4,5(3H,6H)-dione and 5-(((hydroxy((hydroxy(phosphonooxy)phosphoryl)oxy)phosphoryl)oxy)methyl) tetrahydrofuran-2,3,4-triyl triacetate for next step. Analogously to General Method J, the intermediate compound (II) 2-(((hydroxy((hydroxyl(phosphonooxy)phosphoryl)oxy)phosphoryl)oxy) methyl)-5-(5-hydroxy-4-oxo-7-propyl-4,5-dihydro-3H-pyrazolo[4,3-d][1,2,3]triazin-3-yl)tetrahydrofuran-3,4-diyl diacetate was prepared from above intermediate com-pound (I) and hydroxyhydrazine for next step. Analogously to General Method E, the title compound was prepared from the intermediate compound (II) 6.51 g (10 mmol) and the solution of methanol/NaOCH₃ 20 mL (1 mol/L). The mixture was reacted for 5 hand added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.58 (s, 2H), 2.44 (t, J=7.1 Hz, 2H), 2.00 (s, 1H), 1.65 (m, 2H), 0.90 (t, J=8.0 Hz, 3H).

### Example 134. Preparation of (5-(7-cyano-5-hydroxy-4-oxo-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(4-(cyanocarbonyl)-5,6-dioxo-5,6-dihydro-1,2,3-triazin-1(4H)-yl)-5-(((hydroxy((hydroxy(phosphonooxy)phosphoryl)oxy)phosphoryl) oxy)methyl)tetrahydrofuran-3,4-diyl diacetate was prepared from 5,6-dioxo-1,4,5,6-tetrahydro-1,2,3-triazine-4-carbonyl cyanide and 5-(((hydroxy((hydroxy(phosphonooxy)phosphoryl)oxy)phosphoryl) oxy)methyl)tetrahydrofuran-2,3,4-triyl triacetate for next step. Analogously to General Method J, the intermediate compound (II) 2-(7-cyano-5-hydroxy-4-oxo-4,5-dihydro-3H-pyrazolo[4,3-d][1,2,3]triazin-3-yl)-5-(((hydroxy((hydroxy(phosphonooxy)phosphoryl)oxy)phosphoryl)oxy)methyl) tetrahydrofuran-3,4-diyl diacetate was prepared from above intermediate compound (I) and hydroxyhydrazine for next step. Analogously to General Method E, the title compound was prepared from the intermediate compound (II) 6.34 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.58 (s, 2H), 2.00 (s, 1H).

### Example 135. Preparation of (3,4-dihydroxy-5-(5-methyl-4-oxo-7-propyl-4,5-dihydro-3H-pyrazolo[4,3-d][1,2,3]triazin-3-yl)tetrahydrofuran-2-yl)methyl tetrahydrogen triphosphate

Analogously to General Method C, the intermediate compound (I) 2-(((hydroxy((hydroxyl(phosphonooxy)phosphoryl)oxy)phosphoryl)oxy)methyl)-5-(5-methyl-4-oxo-7-propyl-4,5-dihydro-3H-pyrazolo[4,3-d][1,2,3]triazin-3-yl)tetrahydrofuran-3,4-diyl diacetate was prepared from 5-methyl-7-propyl-3H-pyrazolo[4,3-d][1,2,3]triazin-4(5H)-one and 5-(((hydroxy((hydroxy(phosphonooxy) phosphoryl)oxy)phosphoryl)oxy)methyl)tetrahydrofuran-2,3,4-triyl triacetate for next step. Analogously to General Method E, the title compound was prepared from the intermediate compound (I) 6.49 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.95 (s, 3H), 3.58 (s, 2H), 2.44 (t, J=7.1 Hz, 2H), 1.65 (m, 2H), 0.90 (t, J=8.0 Hz, 3H).

### Example 136. Preparation of (5-(7-amino-5-methyl-4-oxo-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(4-carbamoyl-5,6-dioxo-5,6-dihydro-1,2,3-triazin-1(4H)-yl)-5-(((hydroxy((hydroxy(phosphonooxy)phosphoryl)oxy)phosphoryl)oxy) methyl)tetrahydro-furan-3,4-diyl diacetate was prepared from 5,6-dioxo-1,4,5,6-tetrahydro-1,2,3-triazine-4-carboxamide and 5-(((hydroxy((hydroxy(phosphonooxy) phosphoryl)oxy)phosphoryl)oxy) methyl)tetrahydrofuran-2,3,4-triyl triacetate for next step. Analogously to General Method J, the intermediate compound (II) 2-(7-amino-5-methyl-4-oxo-4,5-dihydro-3H-pyrazolo[4,3-d][1,2,3]triazin-3-yl)-5-(((hydroxy((hydroxy(phosphonooxy)phosphoryl)oxy)phosphoryl)oxy)methyl)tetrahydrofuran-3,4-diyl diacetate was prepared from above intermediate compound (I) and hydroxyhydrazine for next step. Analogously to General Method E, the title compound was prepared from the intermediate compound (II) 6.22 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 7.74 (s, 2H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.95 (s, 3H), 3.58 (s, 2H).

### Example 137. Preparation of (5-(7-cyano-5-methyl-4-oxo-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(7-cyano-5-methyl-4-oxo-4,5-dihydro-3H-pyrazolo[4,3-d][1,2,3]triazin-3-yl)-5-(((hydroxy((hydroxy(phosphonooxy)phosphoryl)oxy) phosphoryl)oxy)methyl)tetrahydrofuran-3,4-diyl diacetate was prepared from 5-methyl-4-oxo-4,5-dihydro-3H-pyrazolo[4,3-d][1,2,3]triazine-7-carbonitrile and 5-(((hydroxy((hydroxy(phosphonooxy) phosphoryl)oxy)phosphoryl)oxy)methyl)tetrahydrofuran-2,3,4-triyl triacetate for next step. Analogously to General Method E, the title compound was prepared from the intermediate compound (I) 6.32 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.95 (s, 3H), 3.58 (s, 2H).

### Example 138. Preparation of (3,4-dihydroxy-5-(5-methyl-7-nitro-4-oxo-4,5-dihydro-3H-pyrazolo[4,3-d][1,2,3]triazin-3-yl)tetrahydrofuran-2-yl)methyl tetrahydrogen triphosphate

Analogously to General Method C, the intermediate compound (I) 2-(((hydroxy((hydroxyl(phosphonooxy)phosphoryl)oxy)phosphoryl)oxy)methyl)-5-(5-methyl-7-nitro-4-oxo-4,5-dihydro-3H-pyrazolo[4,3-d][1,2,3]triazin-3-yl)tetrahydrofuran-3,4-diyl diacetate was prepared from 5-methyl-7-nitro-3H-pyrazolo[4,3-d][1,2,3]triazin-4(5H)-one and 5-(((hydroxy((hydroxy(phosphonooxy)phosphoryl) oxy)phosphoryl)oxy)methyl)tetrahydrofuran-2,3,4-triyl triacetate for next step. Analogously to General Method E, the title compound was prepared from the intermediate compound (I) 6.52 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 11.98 (s, 4H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.95 (s, 3H), 3.58 (s, 2H).

### Example 139. Preparation of (5-(5-carbamoyl-4-oxo-7-propyl-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(4-butyryl-5,6-dioxo-5,6-dihydro-1,2,3-triazin-1(4H)-yl)-5-(((hydroxy((hydroxy(phosphonooxy)phosphoryl)oxy)phosphoryl)oxy) methyl)tetrahydro-furan-3,4-diyl diacetate was prepared from 6-butyryl-1,2,3-triazine-4,5(3H,6H)-dione and 5-(((hydroxy((hydroxy(phosphonooxy) phosphoryl)oxy)phosphoryl)oxy)methyl) tetrahydrofuran-2,3,4-triyl triacetate for next step. Analogously to General Method J, the intermediate compound (II) 2-(5-carbamoyl-4-oxo-7-propyl-4,5-dihydro-3H-pyrazolo[4,3-d][1,2,3]triazin-3-yl)-5-(((hydroxy((hydroxyl(phosphonooxy)phosphoryl)oxy)phosphoryl)oxy)methyl) tetrahydrofuran-3,4-diyl diacetate was prepared from above intermediate compound (I) and hydrazinecarboxamide for next step. Analogously to General Method E, the title compound was prepared from the intermediate compound (II) 6.78 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 7.68 (s, 2H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.58 (s, 2H), 2.44 (t, J=7.1 Hz, 2H), 1.65 (m, 2H), 0.90 (t, J=8.0 Hz, 3H).

### Example 140. Preparation of (5-(5-carbamimidoyl-4-oxo-7-propyl-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(4-butyryl-5,6-dioxo-5,6-dihydro-1,2,3-triazin-1(4H)-yl)-5-(((hydroxy((hydroxy(phosphonooxy)phosphoryl)oxy)phosphoryl)oxy) methyl)tetrahydro-furan-3,4-diyl diacetate was prepared from 6-butyryl-1,2,3-triazine-4,5(3H,6H)-dione and 5-(((hydroxy((hydroxy(phosphonooxy) phosphoryl)oxy)phosphoryl)oxy)methyl) tetrahydrofuran-2,3,4-triyl triacetate for next step. Analogously to General Method J, the intermediate compound (II) 2-(5-carbamimidoyl-4-oxo-7-propyl-4,5-dihydro-3H-pyrazolo[4,3-d][1,2,3]triazin-3-yl)-5-(((hydroxy((hydroxy(phosphonooxy)phosphoryl)oxy)phosphoryl)oxy)methyl) tetrahydrofuran-3,4-diyl diacetate was prepared from above intermediate compound (I) and hydrazinecarboximidamide for next step. Analogously to General Method E, the title compound was prepared from the intermediate compound (II) 6.77 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 8.56 (s, 2H), 8.03 (s, 1H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.58 (s, 2H), 2.44 (t, J=7.1 Hz, 2H), 1.65 (m, 2H), 0.90 (t, J=8.0 Hz, 3H).

### Example 141. Preparation of (5-(5-carbamothioyl-4-oxo-7-propyl-4,5-dihydro-3H-pyrazolo[4,3-d][1,2,3]triazin-3-yl)-3,4-dihydroxytetrahydrofuran-2-yl)methyl tetrahydrogen triphosphate

Analogously to General Method C, the intermediate compound (I) 2-(4-butyryl-5,6-dioxo-5,6-dihydro-1,2,3-triazin-1(4H)-yl)-5-(((hydroxy((hydroxy(phosphonooxy)phosphoryl)oxy)phosphoryl)oxy) methyl)tetrahydro-furan-3,4-diyl diacetate was prepared from 6-butyryl-1,2,3-triazine-4,5(3H,6H)-dione and 5-(((hydroxy((hydroxy(phosphonooxy) phosphoryl)oxy)phosphoryl)oxy)methyl) tetrahydrofuran-2,3,4-triyl triacetate for next step. Analogously to General Method J, the intermediate compound (II) 2-(5-carbamothioyl-4-oxo-7-propyl-4,5-dihydro-3H-pyrazolo[4,3-d][1,2,3]triazin-3-yl)-5-(((hydroxy((hydroxy(phosphonooxy)phosphoryl)oxy)phosphoryl)oxy)methyl) tetrahydrofuran-3,4-diyl diacetate was prepared from above intermediate compound (I) and hydrazinecarbothioamide for next step. Analogously to General Method E, the title compound was prepared from the intermediate compound (II) 6.94 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.98 (s, 4H), 8.56 (s, 2H), 5.93 (d, J=7.0 Hz, 1H), 4.51 (t, J=7.0 Hz, 1H), 4.40 (m, J=7.0 Hz, 1H), 4.28 (m, J=7.0 Hz, 1H), 4.15 (m, 2H), 3.58 (s, 2H), 2.44 (t, J=7.1 Hz, 2H), 1.65 (m, 2H), 0.90 (t, J=8.0 Hz, 3H).

### Example 142. Preparation of ethyl 3-(4-(((2-cyanoethoxy)(diisopropylamino) phosphino)oxy)-5-(hydroxymethyl)tetrahydrofuran-2-yl)-5-hydroxy-4-oxo-4,5-dihydro-3H-pyrazolo[4,3-d][1,2,3]triazine-7-carboxylate

Analogously to General Method C, the intermediate compound (I) ethyl 2-(1-(5-(acetoxymethyl)-4-(((2-cyanoethoxy)(diisopropylamino)phosphino)oxy)tetrahydrofuran-2-yl)-5,6-dioxo-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and (5-acetoxy-3-(((2-cyanoethoxy)(diisopropylamino) phosphino)oxy)tetrahydrofuran-2-yl)methyl acetate for next step. Analogously to General Method J, the intermediate compound (II) ethyl 3-(5-(acetoxymethyl)-4-(((2-cyanoethoxy) (diisopropylamino)phosphino)oxy)tetrahydrofuran-2-yl)-5-hydroxy-4-oxo-4,5-dihydro-3H-pyrazolo[4,3-d][1,2,3]triazine-7-carboxylate was prepared from above intermediate compound (I) and hydroxyhydrazine for next step. Analogously to General Method E, the title compound was prepared from the intermediate compound (II) 5.83 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.85 (t, J=7.0 Hz, 1H), 4.40 (m, J=7.0 Hz, 1H), 4.30 (m, J=8.0 Hz, 2H), 3.90 (m, 2H), 3.66 (m, 2H), 3.65 (s, 1H), 3.57 (m, 1H), 2.97 (m, J=6.8 Hz, 2H), 2.60 (t, J=7.1 Hz, 2H), 2.22 (m, 2H), 2.00 (s, 1H), 1.29 (t, J=8.0 Hz, 3H), 1.07 (d, J=6.8 Hz, 12H).

### Example 143. Preparation of 5-(7-amino-5-hydroxy-4-oxo-4,5-dihydro-3H-pyrazolo[4,3-d][1,2,3]triazin-3-yl)-2-(hydroxymethyl)tetrahydrofuran-3-yl (2-cyanoethyl) diisopropylphosphoramidite

Analogously to General Method C, the intermediate compound (I) ethyl (5-(4-cyano-5,6-dioxo-5,6-dihydro-1,2,3-triazin-1(4H)-yl)-3-(((2-cyanoethoxy)(diisopropylamino)phosphino)oxy) tetrahydrofuran-2-yl)methyl acetate was prepared from 5,6-dioxo-1,4,5,6-tetrahydro-1,2,3-triazine-4-carbonitrile and (5-acetoxy-3-(((2-cyanoethoxy)(diisopropylamino)phosphino)oxy)tetrahydrofuran-2-yl) methyl acetate for next step. Analogously to General Method J, the intermediate compound (II) (5-(7-amino-5-hydroxy-4-oxo-4,5-dihydro-3H-pyrazolo[4,3-d][1,2,3]triazin-3-yl)-3-(((2-cyanoethoxy)(diisopropylamino)phosphino)oxy)tetrahydrofuran-2-yl)methyl acetate was prepared from above intermediate compound (I) and hydroxy-hydrazine for next step. Analogously to General Method E, the title compound was prepared from the intermediate compound (II) 5.26 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 7.74 (s, 2H), 5.85 (t, J=7.0 Hz, 1H), 4.40 (m, J=7.0 Hz, 1H), 3.90 (m, 2H), 3.66 (m, 2H), 3.65 (s, 1H), 3.57 (m, 1H), 2.97 (m, J=6.8 Hz, 2H), 2.60 (t, J=7.1 Hz, 2H), 2.22 (m, 2H), 2.00 (s, 1H), 1.07 (d, J=6.8 Hz, 12H).

### Example 144. Preparation of 2-cyanoethyl (2-(hydroxymethyl)-5-(5-methyl-4-oxo-7-propyl-4,5-dihydro-3H-pyrazolo[4,3-d][1,2,3]triazin-3-yl)tetrahydrofuran-3-yl) diisopropylphosphoramidite

Analogously to General Method C, the intermediate compound (I) (3-(((2-cyanoethoxy) (diisopropylamino)phosphino)oxy)-5-(5-methyl-4-oxo-7-propyl-4,5-dihydro-3H-pyrazolo[4,3-d][1,2,3]triazin-3-yl)tetrahydro-furan-2-yl)methyl acetate was prepared from 5-methyl-7-propyl-3H-pyrazolo[4,3-d][1,2,3]triazin-4(5H)-one and (5-acetoxy-3-(((2-cyanoethoxy)(diisopropylamino)phosphino)oxy) tetrahydrofuran-2-yl)methyl acetate for next step. Analogously to General Method E, the title compound was prepared from the intermediate compound (I) 5.51 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.85 (t, J=7.0 Hz, 1H), 4.40 (m, J=7.0 Hz, 1H), 3.95 (s, 3H), 3.90 (m, 2H), 3.66 (m, 2H), 3.65 (s, 1H), 3.57 (m, 1H), 2.97 (m, J=6.8 Hz, 2H), 2.60 (t, J=7.1 Hz, 2H), 2.44 (t, J=7.1 Hz, 2H), 2.22 (m, 2H), 1.65 (m, 2H), 1.07 (d, J=6.8 Hz, 12H), 0.90 (t, J=8.0 Hz, 3H).

### Example 145. Preparation of 2-cyanoethyl (2-(hydroxymethyl)-5-(5-(hydroxymethyl)-4-oxo-7-propyl-4,5-dihydro-3H-pyrazolo [4,3-d][1,2,3]triazin-3-yl)tetrahydrofuran-3-yl) diisopropylphosphoramidite

Analogously to General Method C, the intermediate compound (I) ethyl (5-(4-butyryl-5,6-dioxo-5,6-dihydro-1,2,3-triazin-1(4H)-yl)-3-(((2-cyanoethoxy)(diisopropylamino)phosphino)oxy) tetrahydrofuran-2-yl)methyl acetate was prepared from 6-butyryl-1,2,3-triazine-4,5(3H,6H)-dione and (5-acetoxy-3-(((2-cyanoethoxy)(diisopropylamino)phosphino)oxy)tetrahydrofuran-2-yl)methylacetate for next step. Analogously to General Method J, the intermediate compound (II) (3-(((2-cyanoethoxy) (diisopropylamino)phosphino)oxy)-5-(5-(hydroxymethyl)-4-oxo-7-propyl-4,5-dihydro-3H-pyrazolo[4,3-d][1,2,3]triazin-3-yl)tetrahydrofuran-2-yl)methyl acetate was prepared from above intermediate compound (I) and hydrazinylmethanol for next step. Analogously to General Method E, the title compound was prepared from the intermediate compound (II) 5.67 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.85 (t, J=7.0 Hz, 1H), 4.40 (m, J=7.0 Hz, 1H), 3.90 (m, 2H), 3.66 (m, 2H), 3.65 (s, 2H), 3.57 (m, 1H), 2.97 (m, J=6.8 Hz, 2H), 2.60 (t, J=7.1 Hz, 2H), 2.44 (t, J=7.1 Hz, 2H), 2.22 (m, 2H), 1.65 (m, 2H), 1.07 (d, J=6.8 Hz, 12H), 0.90 (t, J=8.0 Hz, 3H).

### Example 146. Preparation of 2-cyanoethyl (2-(hydroxymethyl)-5-(5-(hydroxymethyl)-7-nitro-4-oxo-4,5-dihydro-3H-pyrazolo [4,3-d][1,2,3]triazin-3-yl)tetrahydrofuran-3-yl) diisopropylphosphoramidite

Analogously to General Method C, the intermediate compound (I) ethyl (3-(((2-cyanoethoxy) (diisopropyl-amino)phosphino)oxy)-5-(4-(nitrocarbonyl)-5,6-dioxo-5,6-dihydro-1,2,3-triazin-1(4H)-yl) tetrahydrofuran-2-yl)methyl acetate was prepared from 6-(nitrocarbonyl)-1,2,3-triazine-4,5(3H,6H)-dione and (5-acetoxy-3-(((2-cyanoethoxy)(diisopropylamino)phosphino)oxy)tetrahydrofuran-2-yl) methylacetate for next step. Analogously to General Method J, the intermediate compound (II) (3-(((2-cyanoethoxy)(diisopropylamino)phosphino)oxy)-5-(5-(hydroxymethyl)-7-nitro-4-oxo-4,5-dihydro-3H-pyrazolo[4,3-d][1,2,3]triazin-3-yl)tetrahydrofuran-2-yl)methyl acetate was prepared from above intermediate compound (I) and hydrazinylmethanol for next step. Analogously to General Method E, the title compound was prepared from the intermediate compound (II) 5.70 g (10 mm ol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.85 (t, J=7.0 Hz, 1H), 4.40 (m, J=7.0 Hz, 1H), 3.90 (m, 2H), 3.66 (m, 2H), 3.65 (s, 2H), 3.57 (m, 1H), 2.97 (m, J=6.8 Hz, 2H), 2.60 (t, J=7.1 Hz, 2H), 2.22 (m, 2H), 1.07 (d, J=6.8 Hz, 12H).

### Example 147. Preparation of 6-(3,4-dihydroxy-5-(hydroxymethyl)tetrahydrofuran-2-yl)-1-propyl-1H-[1,2,3]triazolo[4,5-d][1,2,3]triazin-7(6H)-one

Analogously to General Method C, the intermediate compound (I) 2-(acetoxymethyl)-5-(7-oxo-1-propyl-1H-[1,2,3]triazolo[4,5-d][1,2,3]triazin-6(7H)-yl)tetrahydrofuran-3,4-diyl diacetate was prepared from 1-propyl-1H-[1,2,3]triazolo[4,5-d][1,2,3]triazin-7(6H)-one and -(acetoxymethyl)tetrahydrofuran-2,3,4-triyl triacetate for next step. Analogously to General Method E, the title compound was prepared from the intermediate compound (I) 4.38 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.46 (t, J=7.1 Hz, 2H), 4.40 (m, J=7.0 Hz, 1H), 4.28 (t, J=7.0 Hz, 1H), 3.66 (m, 2H), 3.65 (s, 1H), 3.58 (s, 2H), 1.70 (m, 2H), 0.90 (t, J=8.0 Hz, 3H).

### Example 148. Preparation of 6-(3,4-dihydroxy-5-(hydroxymethyl)tetrahydrofuran-2-yl)-1-(hydroxymethyl)-1H-[1,2,3]triazolo[4,5-d][1,2,3]triazin-7(6H)-one

Analogously to General Method C, the intermediate compound (I) 2-(acetoxymethyl)-5-(1-(acetoxymethyl)-7-oxo-1H-[1,2,3]triazolo[4,5-d][1,2,3]triazin-6(7H)-yl)tetrahydrofuran-3,4-diyl diacetate was prepared from (7-oxo-6,7-dihydro-1H-[1,2,3]triazolo[4,5-d][1,2,3]triazin-1-yl)methyl acetate and 5-(acetoxymethyl)tetrahydrofuran-2,3,4-triyl triacetate for next step. Analogously to General Method E, the title compound was prepared from the intermediate compound (I) 4.68 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 2H), 3.58 (s, 2H).

### Example 149. Preparation of ethyl 2-(6-(3,4-dihydroxy-5-(hydroxymethyl) tetrahydrofuran-2-yl)-7-oxo-6,7-dihydro-1H-[1,2,3]triazolo[4,5-d][1,2,3]triazin-1-yl)acetate

Analogously to General Method C, the intermediate compound (I) 2-(acetoxymethyl)-5-(1-(2-ethoxy-2-oxoethyl)-7-oxo-1H-[1,2,3]triazolo[4,5-d][1,2,3]triazin-6(7H)-yl)tetrahydrofuran-3,4-diyl diacetate was prepared from ethyl 2-(7-oxo-6,7-dihydro-1H-[1,2,3]triazolo[4,5-d][1,2,3]triazin-1-yl) acetate and 5-(acetoxymethyl)tetrahydrofuran-2,3,4-triyl triacetate for next step. Analogously to General Method E, the title compound was prepared from the intermediate compound (I) 4.82 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.93 (d, J=7.0 Hz, 1H), 5.12 (s, 2H), 4.51 (t, J=7.0 Hz, 1H), 4.40 (m, J=7.0 Hz, 1H), 4.28 (t, J=7.0 Hz, 1H), 4.13 (d, J=8.0 Hz, 2H), 3.66 (m, 2H), 3.65 (s, 1H), 3.58 (s, 2H), 1.29 (t, J=8.0 Hz, 3H).

### Example 150. Preparation of 2-(bis(isopropylamino)phosphoryl)-2-((5-(7-oxo-1-propyl-1H-[1,2,3]triazolo[4,5-d][1,2,3]triazin-6(7H)-yl)tetrahydrofuran-2-yl)methoxy)acetic acid

Analogously to General Method C, the intermediate compound (I) 2-(bis(isopropylamino) phosphoryl)-2-((5-(7-oxo-1-propyl-1H-[1,2,3]triazolo[4,5-d][1,2,3]triazin-6(7H)-yl)tetrahydrofuran-2-yl) methoxy)acetic acid was prepared from 1-propyl-1H-[1,2,3]triazolo[4,5-d][1,2,3]triazin-7(6H)-one an d2-((5-acetoxy-tetrahydrofuran-2-yl)methoxy)-2-(bis(isopropylamino)phosphoryl)acetic acid for next step. Analogously to General Method E, the title compound was prepared from the intermediate compound (I) 5.0 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 11.00 (s, 1H), 5.85 (d, J=7.0 Hz, 1H), 4.46 (t, J=7.1 Hz, 2H), 4.07 (m, J=7.0 Hz, 1H), 3.70 (d, J=11.5 Hz, 1H), 3.50 (m, 2H), 2.97 (m, J=6.8 Hz, 2H), 2.07 (m, 2H), 2.00 (s, 2H), 1.81 (m, 2H), 1.70 (m, 2H), 1.07 (d, J=6.8 Hz, 12H), 0.90 (t, J=8.0 Hz, 3H).

### Example 151. Preparation of 6-(3,4-dihydroxy-5-(hydroxymethyl)tetrahydrofuran-2-yl)-3-propyl-3H-[1,2,3]triazolo[4,5-d][1,2,3]triazin-7(6H)-one

Analogously to General Method C, the intermediate compound (I) 2-(acetoxymethyl)-5-(7-oxo-3-propyl-3H-[1,2,3]triazolo[4,5-d][1,2,3]triazin-6(7H)-yl)tetrahydrofuran-3,4-diyl diacetate was prepared from 3-propyl-3H-[1,2,3]triazolo[4,5-d][1,2,3]triazin-7(6H)-one and -(acetoxymethyl)tetrahydrofuran-2,3,4-triyl triacetate for next step. Analogously to General Method E, the title compound was prepared from the intermediate compound (I) 4.38 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.93 (d, J=7.0 Hz, 1H), 4.51 (t, J=7.0 Hz, 1H), 4.46 (t, J=7.1 Hz, 2H), 4.40 (m, J=7.0 Hz, 1H), 4.28 (t, J=7.0 Hz, 1H), 3.66 (m, 2H), 3.65 (s, 1H), 3.58 (s, 2H), 1.70 (m, 2H), 0.90 (t, J=8.0 Hz, 3H).

### Example 152. Preparation of 6-(3,4-dihydroxy-5-(hydroxymethyl)tetrahydrofuran-2-yl)-3-(hydroxymethyl)-3H-[1,2,3]triazolo[4,5-d][1,2,3]triazin-7(6H)-one

Analogously to General Method C, the intermediate compound (I) 2-(acetoxymethyl)-5-(3-(acetoxymethyl)-7-oxo-3H-[1,2,3]triazolo[4,5-d][1,2,3]triazin-6(7H)-yl)tetrahydrofuran-3,4-diyl diacetate was prepared from 3-(hydroxymethyl)-3H-[1,2,3]triazolo[4,5-d][1,2,3]triazin-7(6H)-one and 5-(acetoxymethyl)tetrahydro-furan-2,3,4-triyl triacetate for next step. Analogously to General Method E, the title compound was prepared from the intermediate compound (I) 4.68 g (10 mmol) and the solution of methanol/NaOCH₃20 mL (1 mol/L). The mixture was reacted for 5 h and added acetic acid to pH˜7. The reaction solution was extracted by EtOAc. The crude was separated by silica gel column chromatography to give the title compound. ¹H NMR (600 MHZ, CDCl₃-d₆): δ 5.97 (s, 2H), 5.93 (d, J=7.0 Hz, 1H), 4.51 (t, J=7.0 Hz, 1H), 4.40 (m, J=7.0 Hz, 1H), 4.28 (t, J=7.0 Hz, 1H), 3.66 (m, 2H), 3.65 (s, 2H), 3.58 (s, 2H).

### Example 153. Preparation of ethyl 2-(6-(3,4-dihydroxy-5-(hydroxymethyl) tetrahydrofuran-2-yl)-7-oxo-6,7-dihydro-3H-[1,2,3]triazolo[4,5-d][1,2,3]triazin-3-yl)acetate

Analogously to General Method C, the intermediate compound (I) 2-(acetoxymethyl)-5-(3-(2-ethoxy-2-oxoethyl)-7-oxo-3H-[1,2,3]triazolo[4,5-d][1,2,3]triazin-6(7H)-yl)tetrahydrofuran-3,4-diyl diacetate was prepared from ethyl 2-(7-oxo-6,7-dihydro-3H-[1,2,3]triazolo[4,5-d][1,2,3]triazin-3-yl) acetate and 5-(acetoxymethyl)tetrahydrofuran-2,3,4-triyl triacetate for next step. Analogously to General Method E, the title compound was prepared from the intermediate compound (I) 4.82 g (10 mmol) and the solution of methanol/NaOCH₃ 20 mL (1 mol/L). The mixture was reacted for 5 h and added acetic acid to pH˜7. The reaction solution was extracted by EtOAc. The crude was separated by silica gel column chromato-graphy to give the title compound. ¹H NMR (600 MHZ, CDCl₃-d₆): δ 5.93 (d, J=7.0 Hz, 1H), 5.12 (s, 2H), 4.51 (t, J=7.0 Hz, 1H), 4.40 (m, J=7.0 Hz, 1H), 4.28 (t, J=7.0 Hz, 1H), 4.13 (d, J=8.0 Hz, 2H), 3.66 (m, 2H), 3.65 (s, 1H), 3.58 (s, 2H), 1.29 (t, J=8.0 Hz, 3H).

### Example 154 Preparation of 2-(bis(isopropylamino)phosphoryl)-2-((5-(7-oxo-3-propyl-3H-[1,2,3]triazolo[4,5-d][1,2,3]triazin-6(7H)-yl)tetrahydrofuran-2-yl)methoxy)acetic acid

Analogously to General Method C, the title compound was prepared from 3-propyl-3H-[1,2,3]triazolo[4,5-d][1,2,3]triazin-7(6H)-one 1.80 g (10 mmol) and 2-((5-acetoxytetrahydrofuran-2-yl) methoxy)-2-(bis(isopropylamino)phosphoryl)acetic acid 3.80 g (10 mmol). ¹H NMR (600 MHZ, CDCl₃-d₆): δ 11.00 (s, 1H), 5.85 (d, J=7.0 Hz, 1H), 4.46 (t, J=7.1 Hz, 2H), 4.07 (m, J=7.0 Hz, 1H), 3.70 (d, J=11.5 Hz, 1H), 3.50 (m, 2H), 2.97 (m, J=6.8 Hz, 2H), 2.07 (m, 2H), 2.00 (s, 2H), 1.81 (m, 2H), 1.70 (m, 2H), 1.07 (d, J=6.8 Hz, 12H), 0.90 (t, J=8.0 Hz, 3H).

### Example 155. Preparation of 7-cyclopropyl-3-(3-hydroxy-4-(hydroxymethyl) oxetan-2-yl)-3H-pyrazolo[3,4-d][1,2,3]triazin-4(7H)-one

To a mixture of 2-formylmalonamide 1.30 g (10 mmol) in DMF 25 mL was added cyclopropylhydrazine 0.92 g (11 mmol) in DMF 10 mL, and the mixture was stirred at 120° C. for overnight. The crude product was purified by flash chromatography to give the cyclized pyrazolecarboxamide compound for next step. Analogously to General Method B, the compound of 7-cyclopropyl-3H-pyrazolo[3,4-d][1,2,3]triazin-4(7H)-one was prepared from above 5-amino-1-cyclopropyl-1H-pyrazole-4-carboxamide and NaNO₂. Analogously to General Method C, the title compound was prepared from above 7-cyclopropyl-3H-pyrazolo[3,4-d][1,2,3]triazin-4(7H)-one and 4-(acetoxymethyl)oxetane-2,3-diyl diacetate. ¹H NMR (600 MHZ, CDCl₃-d₆): δ 8.30 (s, 1H), 6.91 (d, J=7.0 Hz, 1H), 5.06 (t, J=7.0 Hz, 1H), 4.89 (m, J=7.0 Hz, 1H), 3.66 (m, 2H), 3.65 (s, 1H), 3.58 (s, 1H), 2.42 (m, J=7.0 Hz, 1H), 0.66 (m, 4H).

### Example 156. Preparation of 7-cyclopropyl-3-(3-(hydroxymethyl)oxiran-2-yl)-3H-pyrazolo [3,4-d][1,2,3]triazin-4(7H)-one

To a mixture of 2-formylmalonamide 1.30 g (10 mmol) in DMF 25 mL was added cyclopropylhydrazine 0.92 g (11 mmol) in DMF 10 mL, and the mixture was stirred at 120° C. for overnight. The crude product was purified by flash chromatography to give the cyclized pyrazolecarboxamide compound for next step. Analogously to General Method B, the compound of 7-cyclopropyl-3H-pyrazolo[3,4-d][1,2,3]triazin-4(7H)-one was prepared from above 5-amino-1-cyclopropyl-1H-pyrazole-4-carboxamide and NaNO2. Analogously to General Method C, the title compound was prepared from above 7-cyclopropyl-3H-pyrazolo[3,4-d][1,2,3]triazin-4(7H)-one and 3-(acetoxymethyl)oxiran-2-yl acetate. ¹H NMR (600 MHZ, CDCl₃-d₆): δ 8.30 (s, 1H), 4.63 (d, J=7.0 Hz, 1H), 3.66 (m, 2H), 3.65 (s, 1H), 3.24 (m, J=7.0 Hz, 1H), 2.42 (m, J=7.0 Hz, 1H), 0.66 (m, 4H).

### Example 157. Preparation of 2-(diethoxyphosphoryl)-2-((5-(hydroxymethyl)-4-oxo-3-(3,4,5-trihydroxy-6-(hydroxymethyl)tetrahydro-2H-pyran-2-yl)-4,5-dihydro-3H-pyrazolo [4,3-d][1,2,3]triazin-7-yl)amino)acetic acid

Analogously to General Method C, 2-(diethoxyphosphoryl)-2-(5,6-dioxo-1-(3,4,5-trihydroxy-6-(hydroxymethyl)tetrahydro-2H-pyran-2-yl)-1,4,5,6-tetrahydro-1,2,3-triazine-4-carboxamido)acetic acid was prepared from 2-(diethoxyphosphoryl)-2-(5,6-dioxo-1,4,5,6-tetrahydro-1,2,3-triazine-4-carboxamido)acetic acid and 6-(acetoxymethyl)tetrahydro-2H-pyran-2,3,4,5-tetrayl tetraacetate; Analogously to General Method J and General Method E the title compound was prepared from 2-(diethoxyphosphoryl)-2-(5,6-dioxo-1-(3,4,5-trihydroxy-6-(hydroxymethyl)tetrahydro-2H-pyran-2-yl)-1,4,5,6-tetrahydro-1,2,3-triazine-4-carboxamido)acetic acid and hydrazinylmethanol. ¹H NMR (600 MHZ, CDCl₃-d₆): δ 11.00 (s, 1H), 6.02 (d, J=9.0 Hz, 1H), 5.97 (s, 2H), 5.58 (s, 1H), 5.24 (s, 1H), 5.08 (s, 1H), 4.42 (t, J=8.7 Hz, 1H), 4.07 (m, 4H), 4.00 (s, 1H), 3.97 (s, 1H), 3.40-3.84 (m, 4H), 3.65 (s, 1H), 3.63 (d, J=8.4 Hz, 1H), 3.50 (d, J=11.5 Hz, 1H), 1.29 (t, 6H).

### Example 158. Preparation of 3-(hydroxymethyl)-5-(3,4,5-trihydroxy-6-(hydroxymethyl)tetrahydro-2H-pyran-2-yl)-3H-[1,2,3]triazolo[4,5-d][1,2,3]triazin-4(5H)-one

Analogously to General Method B, 3-(hydroxymethyl)-3H-[1,2,3]triazolo[4,5-d][1,2,3]triazin-4(5H)-one was prepared from 4-amino-1-(hydroxymethyl)-1H-1,2,3-triazole-5-carboxamide and Na₂NO₂. Analogously to General Method C and E, the title compound was prepared from the 3-(hydroxymethyl)-3H-[1,2,3]triazolo[4,5-d][1,2,3]triazin-4(5H)-one and 6-(acetoxymethyl)tetrahydro-2H-pyran-2,3,4,5-tetrayl tetraacetate. ¹H NMR (600 MHZ, CDCl₃-d₆): δ 6.02 (d, J=9.0 Hz, 1H), 5.97 (s, 2H), 5.58 (s, 1H), 5.24 (s, 1H), 5.08 (s, 1H), 4.42 (t, J=8.7 Hz, 1H), 3.97 (s, 1H), 3.40-3.84 (m, 4H), 3.65 (s, 1H), 3.63 (d, J=8.4 Hz, 1H).

### Example 159. Preparation of 2-(diethoxyphosphoryl)-2-(4-oxo-7-propyl-3-(3,4,5-trihydroxy-6-(hydroxymethyl)tetrahydro-2H-pyran-2-yl)-3H-pyrazolo[4,3-d][1,2,3]triazin-5(4H)-yl)acetic acid

Analogously to General Method C, 6-butyryl-3-(3,4,5-trihydroxy-6-(hydroxymethyl)tetrahydro-2H-pyran-2-yl)-1,2,3-triazine-4,5(3H,6H)-dione was prepared from 6-butyryl-1,2,3-triazine-4,5(3H, 6H)-dione and 6-(acetoxymethyl)tetrahydro-2H-pyran-2,3,4,5-tetrayl tetraacetate; Analogously to General Method J and General Method E the title compound was prepared from 6-butyryl-3-(3,4,5-trihydroxy-6-(hydroxymethyl)tetrahydro-2H-pyran-2-yl)-1,2,3-triazine-4,5(3H,6H)-dione and 2-(diethoxyphosphoryl)-2-hydrazinylacetic acid. ¹H NMR (600 MHZ, CDCl₃-d₆): 611.00 (s, 1H), 6.02 (d, J=9.0 Hz, 1H), 5.58 (s, 1H), 5.24 (s, 1H), 5.08 (s, 1H), 4.60 (d, J=11.5 Hz, 1H), 4.42 (t, J=8.7 Hz, 1H), 4.07 (m, 4H), 3.97 (s, 1H), 3.40-3.84 (m, 4H), 3.63 (d, J=8.4 Hz, 1H), 2.44 (t, J=7.1 Hz, 2H), 1.65 (m, 2H), 1.29 (t, 6H), 0.90 (t, J=8.0 Hz, 3H).

### Example 160. Preparation of 5-(hydroxymethyl)-7-propyl-3-(3,4,5-trihydroxy-6-(hydroxymethyl)tetrahydro-2H-pyran-2-yl)-3H-pyrazolo[4,3-d][1,2,3]triazin-4(5H)-one

Analogously to General Method C, 6-butyryl-3-(3,4,5-trihydroxy-6-(hydroxymethyl)tetrahydro-2H-pyran-2-yl)-1,2,3-triazine-4,5(3H,6H)-dione was prepared from 6-butyryl-1,2,3-triazine-4,5(3H, 6H)-dione and 6-(acetoxymethyl)tetrahydro-2H-pyran-2,3,4,5-tetrayl tetraacetate; Analogously to General Method J and General Method E the title compound was prepared from 6-butyryl-3-(3,4,5-trihydroxy-6-(hydroxymethyl)tetrahydro-2H-pyran-2-yl)-1,2,3-triazine-4,5(3H,6H)-dione and hydrazinylmethanol. ¹H NMR (600 MHZ, CDCl₃-d₆): δ 6.02 (d, J=9.0 Hz, 1H), 5.97 (s, 2H), 5.58 (s, 1H), 5.24 (s, 1H), 5.08 (s, 1H), 4.42 (t, J=8.7 Hz, 1H), 3.97 (s, 1H), 3.40-3.84 (m, 4H), 3.65 (s, 1H), 3.63 (d, J=8.4 Hz, 1H), 2.44 (t, J=7.1 Hz, 2H), 1.65 (m, 2H), 0.90 (t, J=8.0 Hz, 3H).

### Example 161. Preparation of 1-methyl-3-propyl-5-(3,4,5-trihydroxy-6-(hydroxymethyl)tetrahydro-2H-pyran-2-yl)-1H-pyrazolo[3,4-d][1,2,3]triazin-4(5H)-one

Analogously to General Method B, 1-methyl-3-propyl-1H-pyrazolo[3,4-d][1,2,3]triazin-4(5H)-one was prepared from 5-amino-1-methyl-3-propyl-1H-pyrazole-4-carboxamide and Na₂NO₂. Analogously to General Method C and E, the title compound was prepared from the 1-methyl-3-propyl-JH-pyrazolo[3,4-d][1,2,3]triazin-4(5H)-one and 6-(acetoxymethyl)tetrahydro-2H-pyran-2,3,4,5-tetrayl tetraacetate. ¹H NMR (600 MHZ, CDCl₃-d₆): δ 6.02 (d, J=9.0 Hz, 1H), 5.58 (s, 1H), 5.24 (s, 1H), 5.08 (s, 1H), 4.42 (t, J=8.7 Hz, 1H), 4.07 (s, 3H), 3.97 (s, 1H), 3.40-3.84 (m, 4H), 3.63 (d, J=8.4 Hz, 1H), 2.44 (t, J=7.1 Hz, 2H), 1.65 (m, 2H), 0.90 (t, J=8.0 Hz, 3H).

### Example 162. Preparation of 1-(hydroxymethyl)-3-propyl-5-(3,4,5-trihydroxy-6-(hydroxymethyl)tetrahydro-2H-pyran-2-yl)-1H-pyrazolo[3,4-d][1,2,3]triazin-4(5H)-one

Analogously to General Method B, 1-(hydroxymethyl)-3-propyl-1H-pyrazolo[3,4-d][1,2,3]triazin-4(5H)-one was prepared from 5-amino-1-(hydroxymethyl)-3-propyl-1H-pyrazole-4-Carboxamide and Na₂NO₂. Analogously to General Method C and E, the title compound was prepared from the 1-(hydroxymethyl)-3-propyl-1H-pyrazolo[3,4-d][1,2,3]triazin-4(5H)-one and 6-(acetoxymethyl) tetrahydro-2H-pyran-2,3,4,5-tetrayl tetraacetate. ¹H NMR (600 MHZ, CDCl₃-d₆): δ 6.02 (d, J=9.0 Hz, 1H), 5.97 (s, 2H), 5.58 (s, 1H), 5.24 (s, 1H), 5.08 (s, 1H), 4.42 (t, J=8.7 Hz, 1H), 3.97 (s, 1H), 3.40-3.84 (m, 4H), 3.65 (s, 1H), 3.63 (d, J=8.4 Hz, 1H), 2.44 (t, J=7.1 Hz, 2H), 1.65 (m, 2H), 0.90 (t, J=8.0 Hz, 3H).

### Example 163. Preparation of 4-methoxy-N-(5-methyl-4-oxo-3-(3,4,5-trihydroxy-6-(hydroxymethyl)tetrahydro-2H-pyran-2-yl)-4,5-dihydro-3H-pyrazolo[4,3-d][1,2,3]triazin-7-yl) benzamide

Analogously to General Method C, N-(4-methoxybenzoyl)-5,6-dioxo-1-(3,4,5-trihydroxy-6-(hydroxymethyl)tetrahydro-2H-pyran-2-yl)-1,4,5,6-tetrahydro-1,2,3-triazine-4-carboxamide was prepared from N-(4-methoxybenzoyl)-5,6-dioxo-1,4,5,6-tetrahydro-1,2,3-triazine-4-carboxamide and 6-(acetoxymethyl)tetrahydro-2H-pyran-2,3,4,5-tetrayl tetraacetate; Analogously to General Method J and General Method E the title compound was prepared from N-(4-methoxybenzoyl)-5,6-dioxo-1-(3,4,5-trihydroxy-6-(hydroxymethyl)tetrahydro-2H-pyran-2-yl)-1,4,5,6-tetrahydro-1,2,3-triazine-4-carboxamide and methylhydrazine. ¹H NMR (600 MHZ, CDCl₃-d₆): δ 9.15 (s, 1H), 7.92 (d, 2H), 7.17 (d, 2H), 6.02 (d, J=9.0 Hz, 1H), 5.58 (s, 1H), 5.24 (s, 1H), 5.08 (s, 1H), 4.42 (t, J=8.7 Hz, 1H), 3.97 (s, 1H), 3.95 (s, 3H), 3.40-3.84 (m, 4H), 3.83 (s, 3H), 3.63 (d, J=8.4 Hz, 1H).

### Example 164. Preparation of 3-(dimethylamino)-N-(5-methyl-4-oxo-3-(3,4,5-trihydroxy-6-(hydroxymethyl)tetrahydro-2H-pyran-2-yl)-4,5-dihydro-3H-pyrazolo[4,3-d][1,2,3]triazin-7-yl)propanamide

Analogously to General Method C, N-(3-(dimethylamino)propanoyl)-5,6-dioxo-1-(3,4,5-trihydroxy-6-(hydroxymethyl)tetrahydro-2H-pyran-2-yl)-1,4,5,6-tetrahydro-1,2,3-triazine-4-carboxamide was prepared from N-(3-(dimethylamino)propanoyl)-5,6-dioxo-1,4,5,6-tetrahydro-1,2,3-triazine-4-carboxamide and 6-(acetoxymethyl)tetrahydro-2H-pyran-2,3,4,5-tetrayl tetraacetate; Analogously to General Method J and General Method E the title compound was prepared from N-(3-(dimethylamino)propanoyl)-5,6-dioxo-1-(3,4,5-trihydroxy-6-(hydroxymethyl)tetrahydro-2H-pyran-2-yl)-1,4,5,6-tetrahydro-1,2,3-triazine-4-carboxamide and methylhydrazine. ¹H NMR (600 MHZ, CDCl₃-d₆): δ 9.15 (s, 1H), 6.02 (d, J=9.0 Hz, 1H), 5.58 (s, 1H), 5.24 (s, 1H), 5.08 (s, 1H), 4.42 (t, J=8.7 Hz, 1H), 3.97 (s, 1H), 3.95 (s, 3H), 3.40-3.84 (m, 4H), 3.65 (t, J=7.1 Hz, 2H), 3.63 (d, J=8.4 Hz, 1H), 2.84 (s, 6H), 2.50 (t, J=7.1 Hz, 2H).

### Example 165. Preparation of 1-(hydroxymethyl)-5-(3,4,5-trihydroxy-6-(hydroxymethyl)tetrahydro-2H-pyran-2-yl)-1H-[1,2,3]triazolo[4,5-d][1,2,3]triazin-4(5H)-one

Analogously to General Method B, 1-(hydroxymethyl)-1H-[1,2,3]triazolo[4,5-d][1,2,3]triazin-4(5H)-one was prepared from 5-amino-1-(hydroxymethyl)-1H-1,2,3-triazole-4-carboxamide and Na₂NO₂. Analogously to General Method C and E, the title compound was prepared from the 1-(hydroxymethyl)-1H-[1,2,3]triazolo[4,5-d][1,2,3]triazin-4(5H)-one and 6-(acetoxymethyl)tetrahydro-2H-pyran-2,3,4,5-tetrayl tetraacetate. ¹H NMR (600 MHZ, CDCl₃-d₆): δ 6.02 (d, J=9.0 Hz, 1H), 5.97 (s, 2H), 5.58 (s, 1H), 5.24 (s, 1H), 5.08 (s, 1H), 4.42 (t, J=8.7 Hz, 1H), 3.97 (s, 1H), 3.40-3.84 (m, 4H), 3.65 (s, 1H), 3.63 (d, J=8.4 Hz, 1H).

### Example 166. Preparation of ethyl 7-carbamoyl-1-oxo-2-(3,4,5-trihydroxy-6-(hydroxymethyl)tetrahydro-2H-pyran-2-yl)-2,7-dihydro-1H-pyrazolo[4,3-d][1,2,3]triazine-5-carboxylate

Analogously to General Method C, ethyl 2-(5,6-dioxo-1-(3,4,5-trihydroxy-6-(hydroxymethyl) tetrahydro-2H-pyran-2-yl)-1,4,5,6-tetrahydro-1,2,3-triazin-4-yl)-2-oxoacetate was prepared from ethyl 2-(5,6-dioxo-1,4,5,6-tetrahydro-1,2,3-triazin-4-yl)-2-oxoacetate and 6-(acetoxymethyl) tetrahydro-2H-pyran-2,3,4,5-tetrayl tetraacetate; Analogously to General Method J and General Method E the title compound was prepared from ethyl 2-(5,6-dioxo-1-(3,4,5-trihydroxy-6-(hydroxymethyl)tetrahydro-2H-pyran-2-yl)-1,4,5,6-tetrahydro-1,2,3-triazin-4-yl)-2-oxoacetate and hydrazinecarboxamide. ¹H NMR (600 MHZ, CDCl₃-d₆): δ 7.68 (s, 2H), 6.02 (d, J=9.0 Hz, 1H), 5.58 (s, 1H), 5.24 (s, 1H), 5.08 (s, 1H), 4.42 (t, J=8.7 Hz, 1H), 4.30 (m, J=8.0 Hz, 2H), 3.97 (s, 1H), 3.40-3.84 (m, 4H), 3.63 (d, J=8.4 Hz, 1H), 1.29 (t, J=8.0 Hz, 3H).

### Example 167. Preparation of 5-methyl-7-propyl-3-(3,4,5-trihydroxy-6-(hydroxymethyl)tetrahydro-2H-pyran-2-yl)-3H-pyrazolo[4,3-d][1,2,3]triazin-4(5H)-one

Analogously to General Method C, 6-butyryl-3-(3,4,5-trihydroxy-6-(hydroxymethyl)tetrahydro-2H-pyran-2-yl)-1,2,3-triazine-4,5(3H,6H)-dione was prepared from 6-butyryl-1,2,3-triazine-4,5(3H, 6H)-dione and 6-(acetoxymethyl)tetrahydro-2H-pyran-2,3,4,5-tetrayl tetraacetate; Analogously to General Method J and General Method E the title compound was prepared from 6-butyryl-3-(3,4,5-trihydroxy-6-(hydroxymethyl)tetrahydro-2H-pyran-2-yl)-1,2,3-triazine-4,5(3H,6H)-dione and methylhydrazine. ¹H NMR (600 MHZ, CDCl₃-d₆): δ 6.02 (d, J=9.0 Hz, 1H), 5.58 (s, 1H), 5.24 (s, 1H), 5.08 (s, 1H), 4.42 (t, J=8.7 Hz, 1H), 3.97 (s, 1H), 3.95 (s, 3H), 3.40-3.84 (m, 4H), 3.63 (d, J=8.4 Hz, 1H), 2.44 (t, J=7.1 Hz, 2H), 1.65 (m, 2H), 0.90 (t, J=8.0 Hz, 3H).

TABLE 1
Example 1-167

| | | | Purity |
|---|---|---|---|
| Example | Chemical Structure | M⁺/e | % |
| 1 | 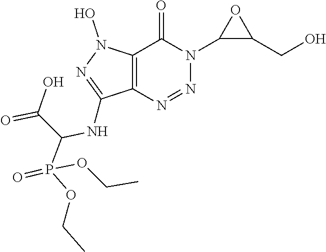 [SMILES: CCOP(=O)(OCC)C(Nc1nn(O)c2c(=O)n(C3OC3CO)nnc12)C(=O)O] | 434.10 | 95.6 |
| 2 | 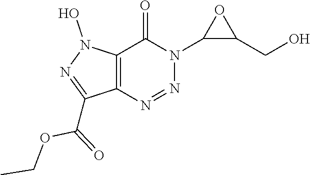 [SMILES: CCOC(=O)c1nn(O)c2c(=O)n(C3OC3CO)nnc12] | 297.07 | 97.2 |
| 3 | 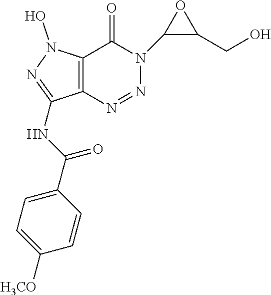 [SMILES: COc1ccc(C(=O)Nc2nn(O)c3c(=O)n(C4OC4CO)nnc23)cc1] | 374.10 | 95.8 |
| 4 | 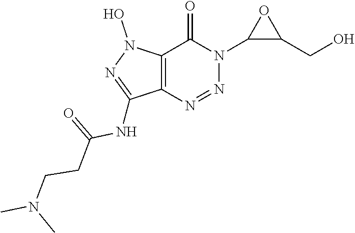 [SMILES: CN(C)CCC(=O)Nc1nn(O)c2c(=O)n(C3OC3CO)nnc12] | 339.13 | 96.9 |
| 5 | 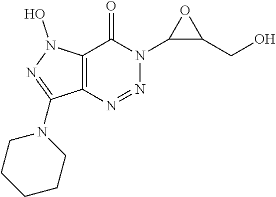 [SMILES: O=c1c2c(nnn1C1OC1CO)c(N1CCCCC1)nn2O] | 308.12 | 95.5 |
| 6 | 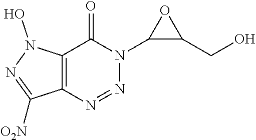 [SMILES: O=c1c2c(nnn1C1OC1CO)c([N+](=O)[O-])nn2O] | 270.03 | 96.1 |
| 7 | 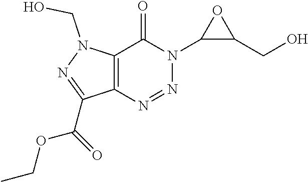 [SMILES: CCOC(=O)c1nn(CO)c2c(=O)n(C3OC3CO)nnc12] | 311.09 | 97.3 |
| 8 | 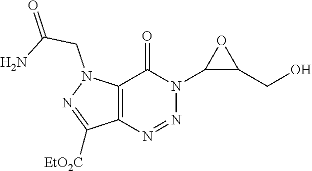 [SMILES: CCOC(=O)c1nn(CC(N)=O)c2c(=O)n(C3OC3CO)nnc12] | 338.10 | 95.3 |
| 9 | 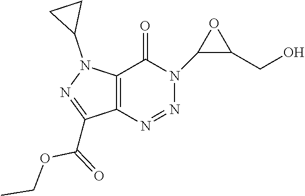 [SMILES: CCOC(=O)c1nn(C2CC2)c2c(=O)n(C3OC3CO)nnc12] | 321.11 | 96.9 |
| 10 | 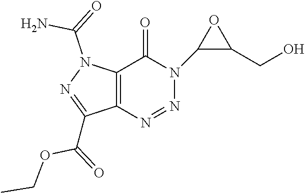 [SMILES: CCOC(=O)c1nn(C(N)=O)c2c(=O)n(C3OC3CO)nnc12] | 324.08 | 95.8 |
| 11 | 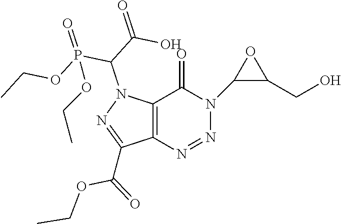 [SMILES: CCOC(=O)c1nn(C(C(=O)O)P(=O)(OCC)OCC)c2c(=O)n(C3OC3CO)nnc12] | 475.11 | 92.6 |
| 12 | 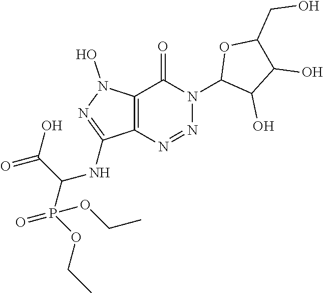 [SMILES: CCOP(=O)(OCC)C(Nc1nn(O)c2c(=O)n(C3OC(CO)C(O)C3O)nnc12)C(=O)O] | 494.12 | 99.3 |
| 13 | 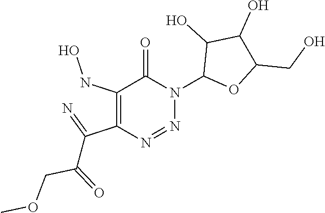 [SMILES: COCC(=O)C(=N)c1nnn(C2OC(CO)C(O)C2O)c(=O)c1NO] | 357.09 | 95.8 |
| 14 | 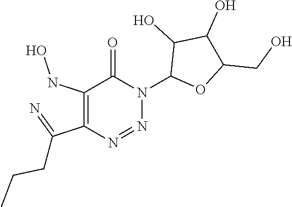 [SMILES: CCCC(=N)c1nnn(C2OC(CO)C(O)C2O)c(=O)c1NO] | 327.12 | 97.6 |
| 15 | 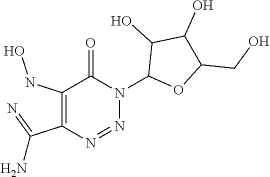 [SMILES: N=C(N)c1nnn(C2OC(CO)C(O)C2O)c(=O)c1NO] | 300.08 | 98.2 |
| 16 | 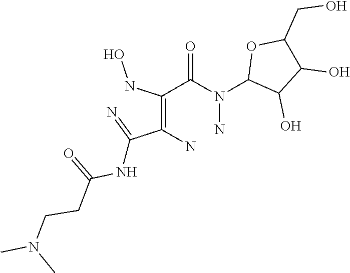 [SMILES: CN(C)CCC(=O)NC(=N)/C(N)=C(\NO)C(=O)N(N)C1OC(CO)C(O)C1O] | 399.15 | 98.2 |
| 17 | 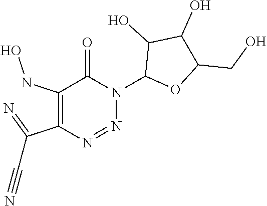 [SMILES: N#CC(=N)c1nnn(C2OC(CO)C(O)C2O)c(=O)c1NO] | 310.07 | 97.8 |
| 18 | 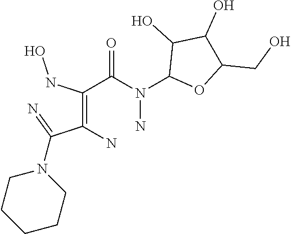 [SMILES: N=C(/C(N)=C(\NO)C(=O)N(N)C1OC(CO)C(O)C1O)N1CCCCC1] | 368.14 | 97.6 |
| 19 | 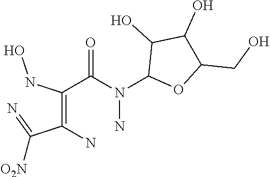 [SMILES: N=C(/C(N)=C(\NO)C(=O)N(N)C1OC(CO)C(O)C1O)[N+](=O)[O-]] | 330.06 | 96.9 |
| 20 | 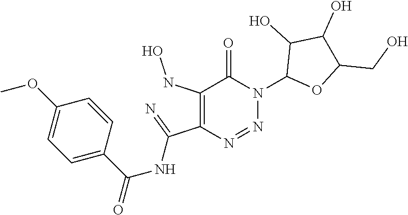 [SMILES: COc1ccc(C(=O)NC(=N)c2nnn(C3OC(CO)C(O)C3O)c(=O)c2NO)cc1] | 434.12 | 98.2 |
| 21 | 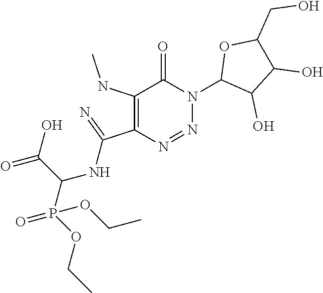 [SMILES: CCOP(=O)(OCC)C(NC(=N)c1nnn(C2OC(CO)C(O)C2O)c(=O)c1NC)C(=O)O] | 492.14 | 95.6 |
| 22 | 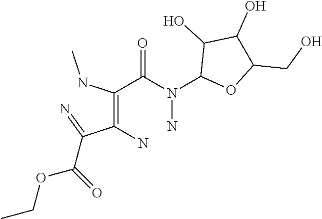 [SMILES: CCOC(=O)C(=N)/C(N)=C(\NC)C(=O)N(N)C1OC(CO)C(O)C1O] | 355.11 | 96.7 |
| 23 | 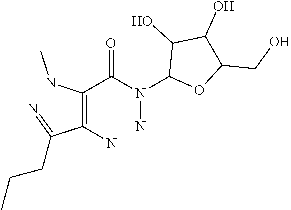 [SMILES: CCCC(=N)/C(N)=C(\NC)C(=O)N(N)C1OC(CO)C(O)C1O] | 325.14 | 97.6 |
| 24 | 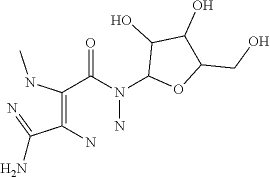 [SMILES: CN/C(C(=O)N(N)C1OC(CO)C(O)C1O)=C(/N)C(=N)N] | 298.10 | 96.9 |
| 25 | 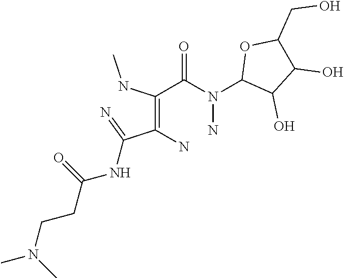 [SMILES: CN/C(C(=O)N(N)C1OC(CO)C(O)C1O)=C(/N)C(=N)NC(=O)CCN(C)C] | 397.17 | 98.1 |
| 26 | 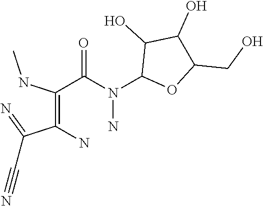 [SMILES: CN/C(C(=O)N(N)C1OC(CO)C(O)C1O)=C(/N)C(=N)C#N] | 308.09 | 99.1 |
| 27 | 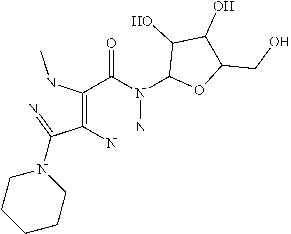 [SMILES: CN/C(C(=O)N(N)C1OC(CO)C(O)C1O)=C(/N)C(=N)N1CCCCC1] | 366.17 | 96.2 |
| 28 | 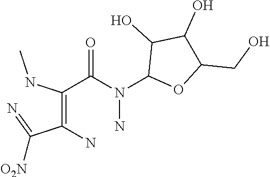 [SMILES: CN/C(C(=O)N(N)C1OC(CO)C(O)C1O)=C(/N)C(=N)[N+](=O)[O-]] | 328.08 | 98.1 |
| 29 | 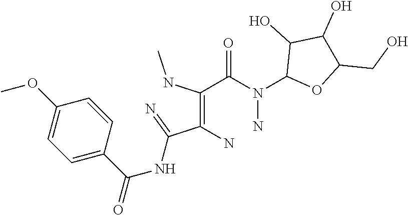 [SMILES: CN/C(C(=O)N(N)C1OC(CO)C(O)C1O)=C(/N)C(=N)NC(=O)c1ccc(OC)cc1] | 432.14 | 97.1 |
| 30 | 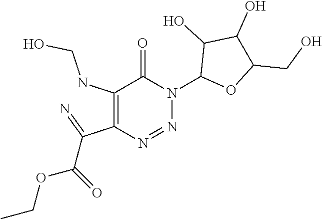 [SMILES: CCOC(=O)C(=N)c1nnn(C2OC(CO)C(O)C2O)c(=O)c1NCO] | 371.11 | 98.6 |
| 31 | 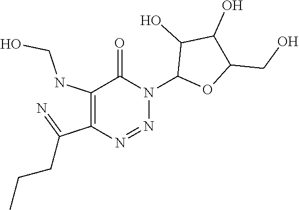 [SMILES: CCCC(=N)c1nnn(C2OC(CO)C(O)C2O)c(=O)c1NCO] | 341.13 | 98.8 |
| 32 | 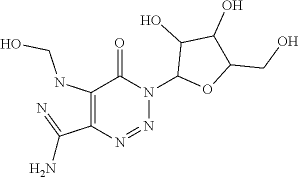 [SMILES: N=C(N)c1nnn(C2OC(CO)C(O)C2O)c(=O)c1NCO] | 314.10 | 98.1 |
| 33 | 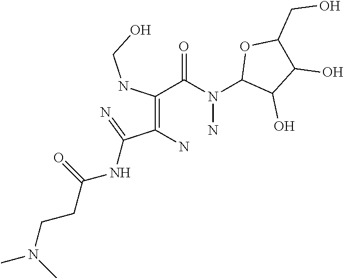 [SMILES: CN(C)CCC(=O)NC(=N)/C(N)=C(\NCO)C(=O)N(N)C1OC(CO)C(O)C1O] | 413.17 | 95.9 |
| 34 | 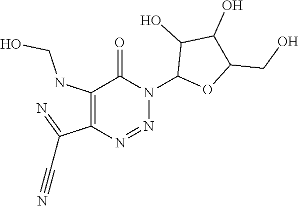 [SMILES: N#CC(=N)c1nnn(C2OC(CO)C(O)C2O)c(=O)c1NCO] | 324.08 | 96.3 |
| 35 | 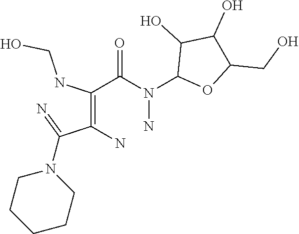 [SMILES: N=C(/C(N)=C(\NCO)C(=O)N(N)C1OC(CO)C(O)C1O)N1CCCCC1] | 382.16 | 96.9 |
| 36 | 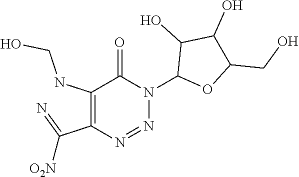 [SMILES: N=C(c1nnn(C2OC(CO)C(O)C2O)c(=O)c1NCO)[N+](=O)[O-]] | 344.07 | 96.7 |
| 37 | 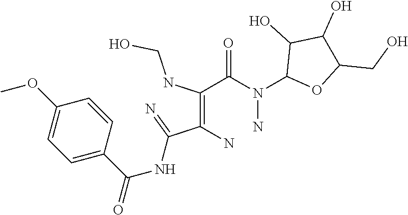 [SMILES: COc1ccc(C(=O)NC(=N)/C(N)=C(\NCO)C(=O)N(N)C2OC(CO)C(O)C2O)cc1] | 448.13 | 95.6 |
| 38 | 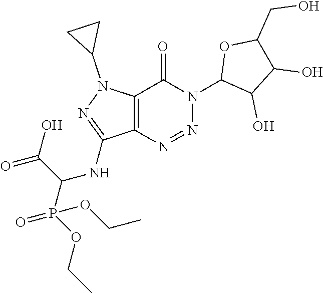 [SMILES: CCOP(=O)(OCC)C(Nc1nn(C2CC2)c2c(=O)n(C3OC(CO)C(O)C3O)nnc12)C(=O)O] | 518.15 | 96.9 |
| 39 | 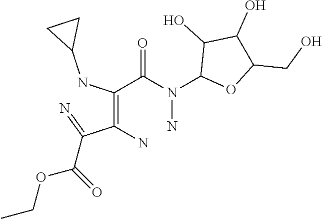 [SMILES: CCOC(=O)C(=N)/C(N)=C(\NC1CC1)C(=O)N(N)C1OC(CO)C(O)C1O] | 381.13 | 95.6 |
| 40 | 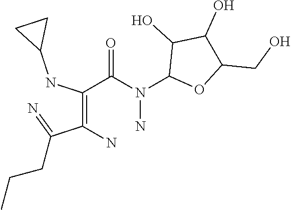 [SMILES: CCCC(=N)/C(N)=C(\NC1CC1)C(=O)N(N)C1OC(CO)C(O)C1O] | 351.15 | 98.0 |
| 41 | 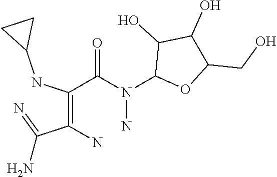 [SMILES: N=C(N)/C(N)=C(\NC1CC1)C(=O)N(N)C1OC(CO)C(O)C1O] | 324.12 | 95.9 |
| 42 | 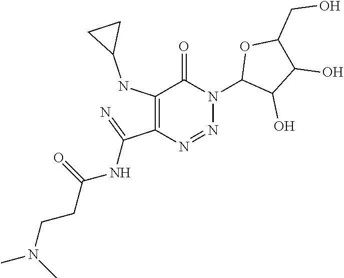 [SMILES: CN(C)CCC(=O)NC(=N)c1nnn(C2OC(CO)C(O)C2O)c(=O)c1NC1CC1] | 423.19 | 96.2 |
| 43 | 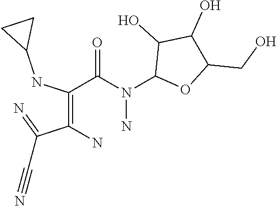 [SMILES: N#CC(=N)/C(N)=C(\NC1CC1)C(=O)N(N)C1OC(CO)C(O)C1O] | 334.10 | 95.9 |
| 44 | 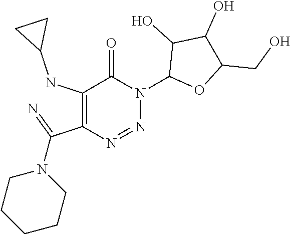 [SMILES: N=C(c1nnn(C2OC(CO)C(O)C2O)c(=O)c1NC1CC1)N1CCCCC1] | 392.18 | 95.6 |
| 45 | 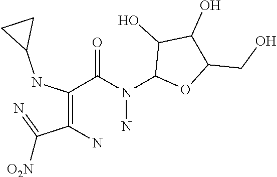 [SMILES: N=C(/C(N)=C(\NC1CC1)C(=O)N(N)C1OC(CO)C(O)C1O)[N+](=O)[O-]] | 354.09 | 96.0 |
| 46 | 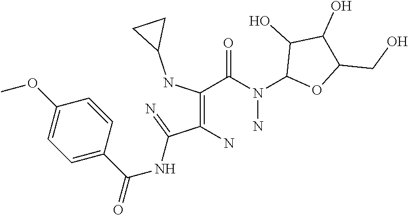 [SMILES: COc1ccc(C(=O)NC(=N)/C(N)=C(\NC2CC2)C(=O)N(N)C2OC(CO)C(O)C2O)cc1] | 458.15 | 96.5 |
| 47 | 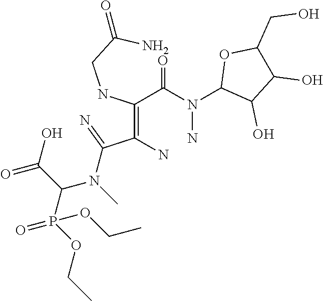 [SMILES: CCOP(=O)(OCC)C(C(=O)O)N(C)C(=N)/C(N)=C(\NCC(N)=O)C(=O)N(N)C1OC(CO)C(O)C1O] | 549.16 | 95.9 |
| 48 | 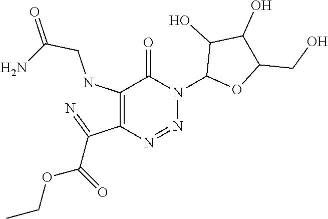 [SMILES: CCOC(=O)C(=N)c1nnn(C2OC(CO)C(O)C2O)c(=O)c1NCC(N)=O] | 398.12 | 98.8 |
| 49 | 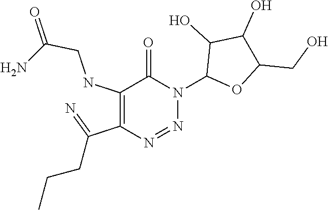 [SMILES: CCCC(=N)c1nnn(C2OC(CO)C(O)C2O)c(=O)c1NCC(N)=O] | 368.14 | 97.4 |
| 50 | 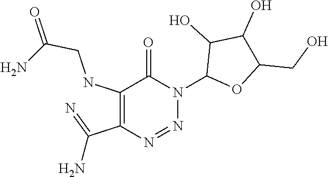 [SMILES: N=C(N)c1nnn(C2OC(CO)C(O)C2O)c(=O)c1NCC(N)=O] | 341.11 | 97.5 |
| 51 | 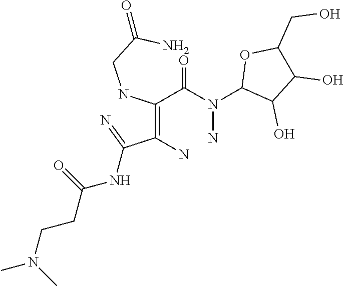 [SMILES: CN(C)CCC(=O)NC(=N)/C(N)=C(\NCC(N)=O)C(=O)N(N)C1OC(CO)C(O)C1O] | 440.18 | 95.2 |
| 52 | 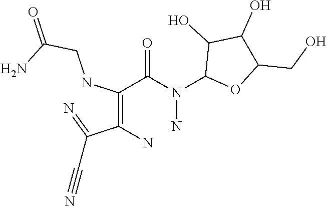 [SMILES: N#CC(=N)/C(N)=C(\NCC(N)=O)C(=O)N(N)C1OC(CO)C(O)C1O] | 351.09 | 95.6 |
| 53 | 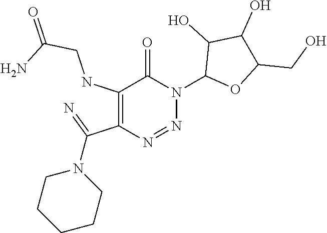 [SMILES: N=C(c1nnn(C2OC(CO)C(O)C2O)c(=O)c1NCC(N)=O)N1CCCCC1] | 409.17 | 96.6 |
| 54 | 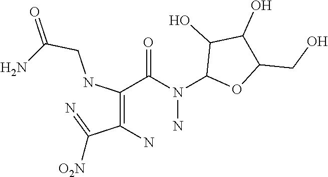 [SMILES: N=C(/C(N)=C(\NCC(N)=O)C(=O)N(N)C1OC(CO)C(O)C1O)[N+](=O)[O-]] | 371.08 | 96.9 |
| 55 | 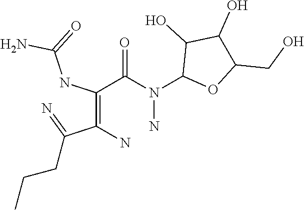 [SMILES: CCCC(=N)/C(N)=C(\NC(N)=O)C(=O)N(N)C1OC(CO)C(O)C1O] | 354.13 | 97.1 |
| 56 | 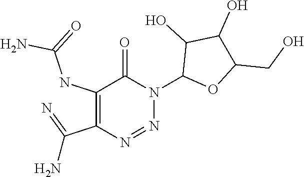 [SMILES: N=C(N)c1nnn(C2OC(CO)C(O)C2O)c(=O)c1NC(N)=O] | 327.09 | 97.9 |
| 57 | 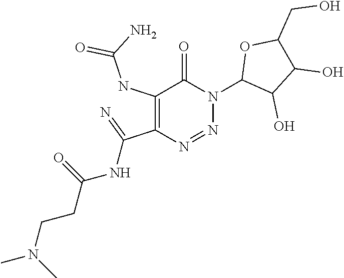 [SMILES: CN(C)CCC(=O)NC(=N)c1nnn(C2OC(CO)C(O)C2O)c(=O)c1NC(N)=O] | 426.16 | 95.9 |
| 58 | 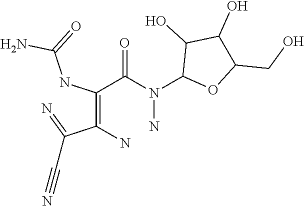 [SMILES: N#CC(=N)/C(N)=C(\NC(N)=O)C(=O)N(N)C1OC(CO)C(O)C1O] | 337.08 | 98.8 |
| 59 | 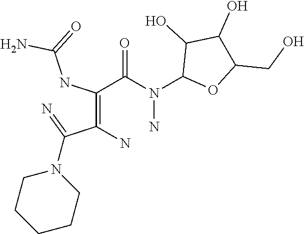 [SMILES: N=C(/C(N)=C(\NC(N)=O)C(=O)N(N)C1OC(CO)C(O)C1O)N1CCCCC1] | 395.16 | 96.9 |
| 60 | 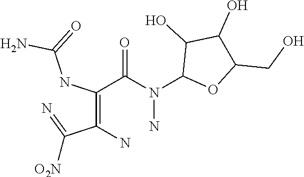 [SMILES: N=C(/C(N)=C(\NC(N)=O)C(=O)N(N)C1OC(CO)C(O)C1O)[N+](=O)[O-]] | 357.07 | 97.3 |
| 61 | 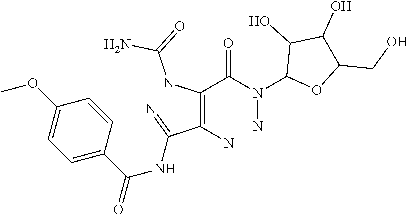 [SMILES: COc1ccc(C(=O)NC(=N)/C(N)=C(\NC(N)=O)C(=O)N(N)C2OC(CO)C(O)C2O)cc1] | 461.13 | 97.4 |
| 62 | 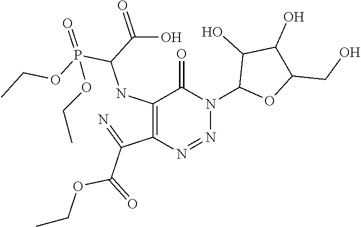 [SMILES: CCOC(=O)C(=N)c1nnn(C2OC(CO)C(O)C2O)c(=O)c1NC(C(=O)O)P(=O)(OCC)OCC] | 535.13 | 96.9 |
| 63 | 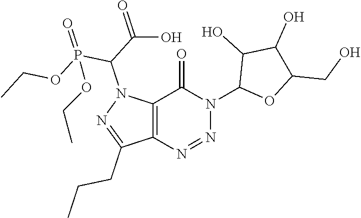 [SMILES: CCCc1nn(C(C(=O)O)P(=O)(OCC)OCC)c2c(=O)n(C3OC(CO)C(O)C3O)nnc12] | 505.16 | 97.0 |
| 64 | 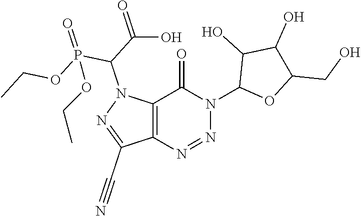 [SMILES: CCOP(=O)(OCC)C(C(=O)O)n1nc(C#N)c2nnn(C3OC(CO)C(O)C3O)c(=O)c21] | 488.11 | 95.9 |
| 65 | 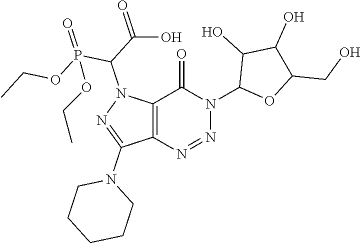 [SMILES: CCOP(=O)(OCC)C(C(=O)O)n1nc(N2CCCCC2)c2nnn(C3OC(CO)C(O)C3O)c(=O)c21] | 546.18 | 96.7 |
| 66 | 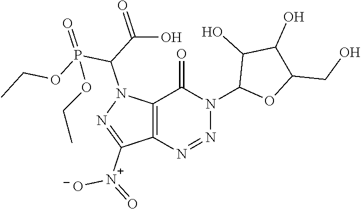 [SMILES: CCOP(=O)(OCC)C(C(=O)O)n1nc([N+](=O)[O-])c2nnn(C3OC(CO)C(O)C3O)c(=O)c21] | 508.10 | 95.6 |
| 67 | 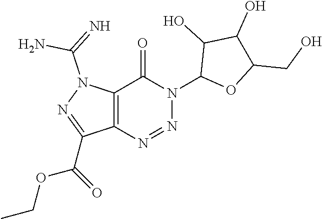 [SMILES: CCOC(=O)c1nn(C(=N)N)c2c(=O)n(C3OC(CO)C(O)C3O)nnc12] | 383.12 | 95.9 |
| 68 | 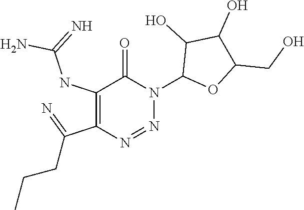 [SMILES: CCCC(=N)c1nnn(C2OC(CO)C(O)C2O)c(=O)c1NC(=N)N] | 353.14 | 99.3 |
| 69 | 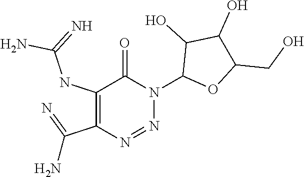 [SMILES: N=C(N)Nc1c(C(=N)N)nnn(C2OC(CO)C(O)C2O)c1=O] | 326.11 | 95.2 |
| 70 | 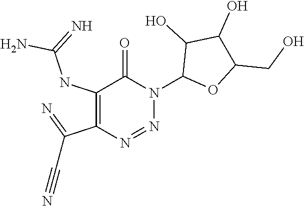 [SMILES: N#CC(=N)c1nnn(C2OC(CO)C(O)C2O)c(=O)c1NC(=N)N] | 336.09 | 98.2 |
| 71 | 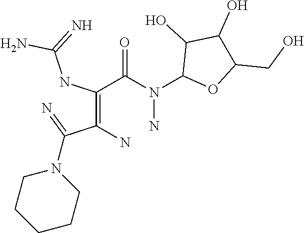 [SMILES: N=C(N)N/C(C(=O)N(N)C1OC(CO)C(O)C1O)=C(/N)C(=N)N1CCCCC1] | 394.17 | 96.9 |
| 72 | 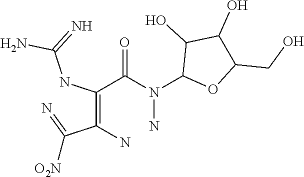 [SMILES: N=C(N)N/C(C(=O)N(N)C1OC(CO)C(O)C1O)=C(/N)C(=N)[N+](=O)[O-]] | 356.08 | 97.6 |
| 73 | 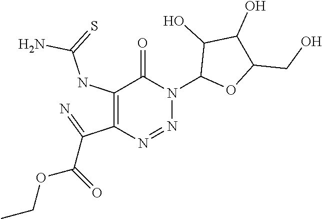 [SMILES: CCOC(=O)C(=N)c1nnn(C2OC(CO)C(O)C2O)c(=O)c1NC(N)=S] | 400.08 | 96.9 |
| 74 | 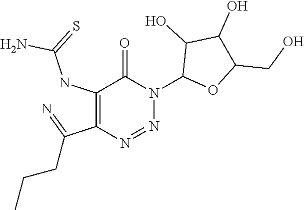 [SMILES: CCCC(=N)c1nnn(C2OC(CO)C(O)C2O)c(=O)c1NC(N)=S] | 370.11 | 98.5 |
| 75 | 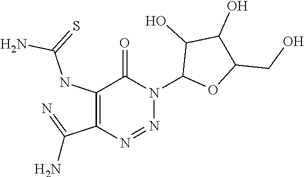 [SMILES: N=C(N)c1nnn(C2OC(CO)C(O)C2O)c(=O)c1NC(N)=S] | 343.07 | 97.2 |
| 76 | 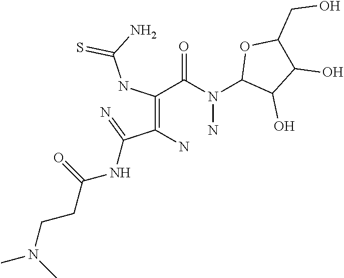 [SMILES: CN(C)CCC(=O)NC(=N)/C(N)=C(\NC(N)=S)C(=O)N(N)C1OC(CO)C(O)C1O] | 442.14 | 99.2 |
| 77 | 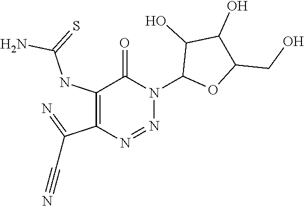 [SMILES: N#CC(=N)c1nnn(C2OC(CO)C(O)C2O)c(=O)c1NC(N)=S] | 353.05 | 98.2 |
| 78 | 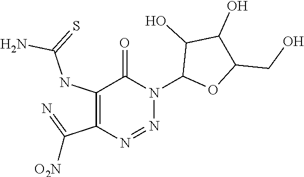 [SMILES: N=C(c1nnn(C2OC(CO)C(O)C2O)c(=O)c1NC(N)=S)[N+](=O)[O-]] | 373.04 | 95.6 |
| 79 | 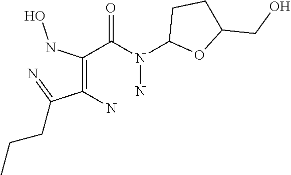 [SMILES: CCCC(=N)/C(N)=C(\NO)C(=O)N(N)C1CCC(CO)O1] | 295.13 | 96.8 |
| 80 | 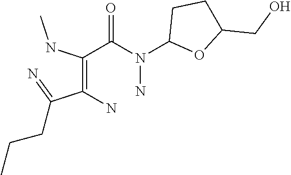 [SMILES: CCCC(=N)/C(N)=C(\NC)C(=O)N(N)C1CCC(CO)O1] | 293.15 | 97.3 |
| 81 | 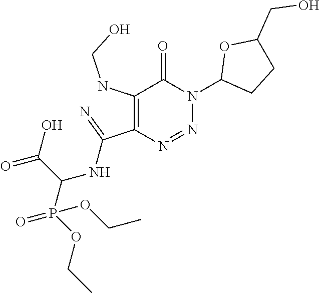 [SMILES: CCOP(=O)(OCC)C(NC(=N)c1nnn(C2CCC(CO)O2)c(=O)c1NCO)C(=O)O] | 476.14 | 96.6 |
| 82 | 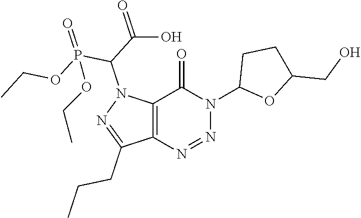 [SMILES: CCCc1nn(C(C(=O)O)P(=O)(OCC)OCC)c2c(=O)n(C3CCC(CO)O3)nnc12] | 473.17 | 98.2 |
| 83 | 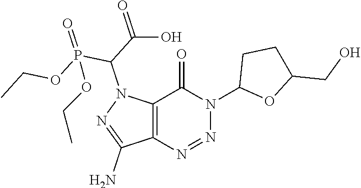 [SMILES: CCOP(=O)(OCC)C(C(=O)O)n1nc(N)c2nnn(C3CCC(CO)O3)c(=O)c21] | 446.13 | 96.7 |
| 84 | 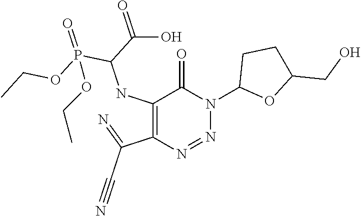 [SMILES: CCOP(=O)(OCC)C(Nc1c(C(=N)C#N)nnn(C2CCC(CO)O2)c1=O)C(=O)O] | 456.12 | 96.9 |
| 85 | 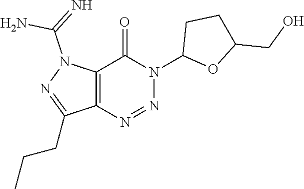 [SMILES: CCCc1nn(C(=N)N)c2c(=O)n(C3CCC(CO)O3)nnc12] | 321.15 | 98.5 |
| 86 | 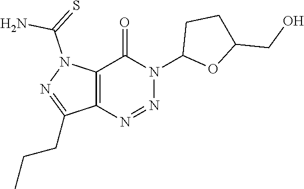 [SMILES: CCCc1nn(C(N)=S)c2c(=O)n(C3CCC(CO)O3)nnc12] | 338.12 | 98.5 |
| 87 | 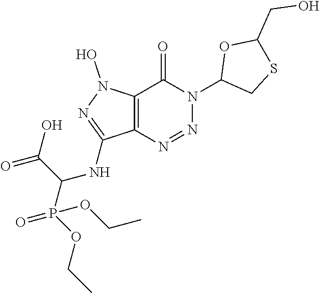 [SMILES: CCOP(=O)(OCC)C(Nc1nn(O)c2c(=O)n(C3CSC(CO)O3)nnc12)C(=O)O] | 480.08 | 95.2 |
| 88 | 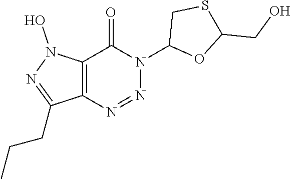 [SMILES: CCCc1nn(O)c2c(=O)n(C3CSC(CO)O3)nnc12] | 313.08 | 96.6 |
| 89 | 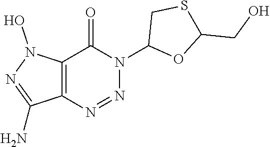 [SMILES: Nc1nn(O)c2c(=O)n(C3CSC(CO)O3)nnc12] | 286.05 | 98.1 |
| 90 | 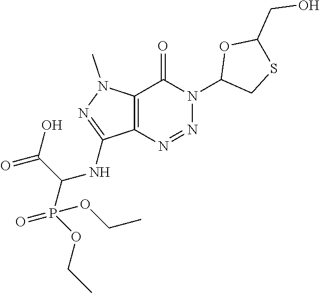 [SMILES: CCOP(=O)(OCC)C(Nc1nn(C)c2c(=O)n(C3CSC(CO)O3)nnc12)C(=O)O] | 478.10 | 97.7 |
| 91 | 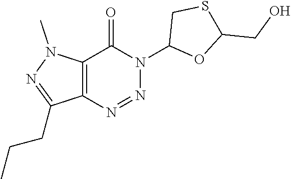 [SMILES: CCCc1nn(C)c2c(=O)n(C3CSC(CO)O3)nnc12] | 311.11 | 96.9 |
| 92 | 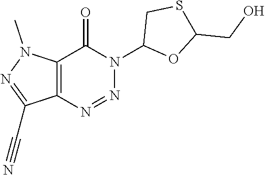 [SMILES: Cn1nc(C#N)c2nnn(C3CSC(CO)O3)c(=O)c21] | 294.05 | 95.3 |
| 93 | 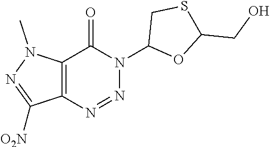 [SMILES: Cn1nc([N+](=O)[O-])c2nnn(C3CSC(CO)O3)c(=O)c21] | 314.04 | 95.9 |
| 94 | 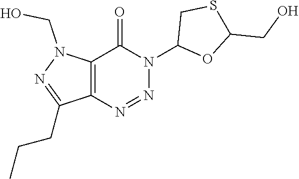 [SMILES: CCCc1nn(CO)c2c(=O)n(C3CSC(CO)O3)nnc12] | 327.10 | 97.6 |
| 95 | 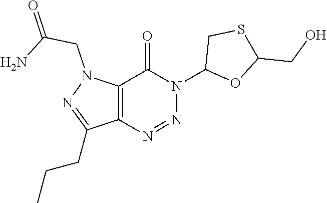 [SMILES: CCCc1nn(CC(N)=O)c2c(=O)n(C3CSC(CO)O3)nnc12] | 354.11 | 99.4 |
| 96 | 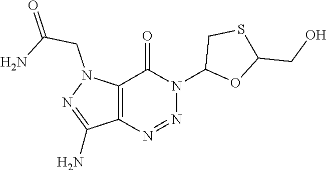 [SMILES: NC(=O)Cn1nc(N)c2nnn(C3CSC(CO)O3)c(=O)c21] | 327.07 | 95.5 |
| 97 | 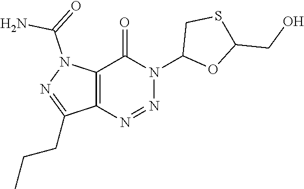 [SMILES: CCCc1nn(C(N)=O)c2c(=O)n(C3CSC(CO)O3)nnc12] | 340.10 | 98.6 |
| 98 | 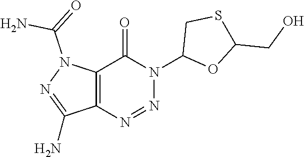 [SMILES: NC(=O)n1nc(N)c2nnn(C3CSC(CO)O3)c(=O)c21] | 313.06 | 97.5 |
| 99 | 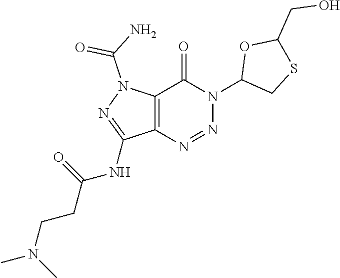 [SMILES: CN(C)CCC(=O)Nc1nn(C(N)=O)c2c(=O)n(C3CSC(CO)O3)nnc12] | 412.13 | 98.1 |
| 100 | 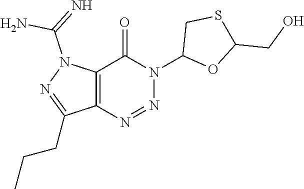 [SMILES: CCCc1nn(C(=N)N)c2c(=O)n(C3CSC(CO)O3)nnc12] | 339.11 | 97.1 |
| 101 | 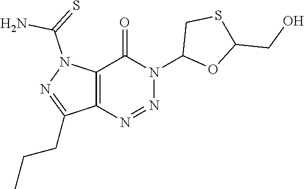 [SMILES: CCCc1nn(C(N)=S)c2c(=O)n(C3CSC(CO)O3)nnc12] | 356.07 | 96.6 |
| 102 | 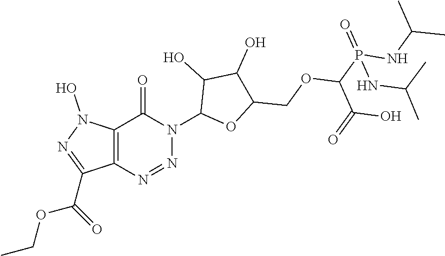 [SMILES: CCOC(=O)c1nn(O)c2c(=O)n(C3OC(COC(C(=O)O)P(=O)(NC(C)C)NC(C)C)C(O)C3O)nnc12] | 577.19 | 95.6 |
| 103 | 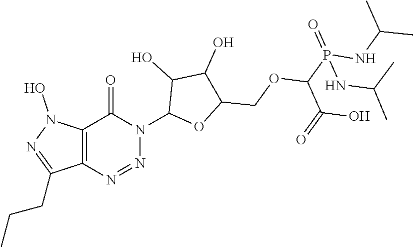 [SMILES: CCCc1nn(O)c2c(=O)n(C3OC(COC(C(=O)O)P(=O)(NC(C)C)NC(C)C)C(O)C3O)nnc12] | 547.22 | 98.2 |
| 104 | 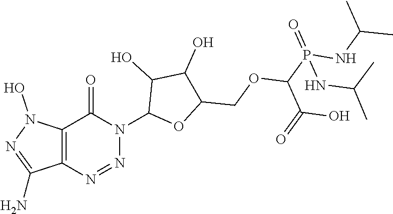 [SMILES: CC(C)NP(=O)(NC(C)C)C(OCC1OC(n2nnc3c(N)nn(O)c3c2=O)C(O)C1O)C(=O)O] | 520.18 | 97.1 |
| 105 | 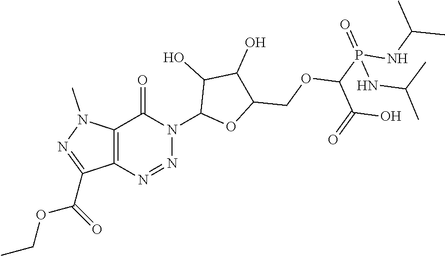 [SMILES: CCOC(=O)c1nn(C)c2c(=O)n(C3OC(COC(C(=O)O)P(=O)(NC(C)C)NC(C)C)C(O)C3O)nnc12] | 575.21 | 95.4 |
| 106 | 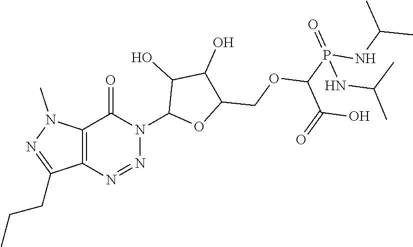 [SMILES: CCCc1nn(C)c2c(=O)n(C3OC(COC(C(=O)O)P(=O)(NC(C)C)NC(C)C)C(O)C3O)nnc12] | 545.24 | 98.9 |
| 107 | 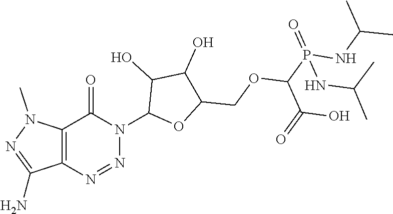 [SMILES: CC(C)NP(=O)(NC(C)C)C(OCC1OC(n2nnc3c(N)nn(C)c3c2=O)C(O)C1O)C(=O)O] | 518.20 | 95.7 |
| 108 | 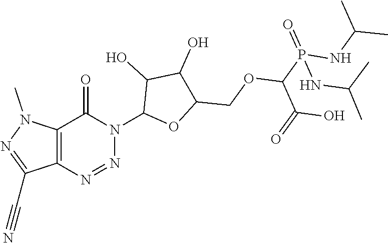 [SMILES: CC(C)NP(=O)(NC(C)C)C(OCC1OC(n2nnc3c(C#N)nn(C)c3c2=O)C(O)C1O)C(=O)O] | 528.18 | 95.9 |
| 109 | 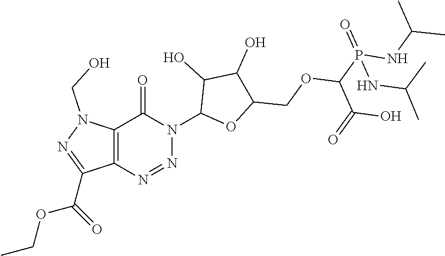 [SMILES: CCOC(=O)c1nn(CO)c2c(=O)n(C3OC(COC(C(=O)O)P(=O)(NC(C)C)NC(C)C)C(O)C3O)nnc12] | 591.21 | 96.7 |
| 110 | 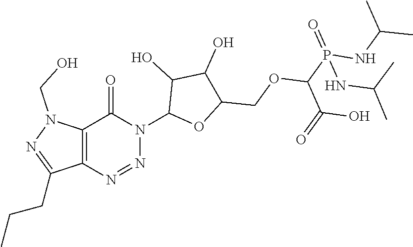 [SMILES: CCCc1nn(CO)c2c(=O)n(C3OC(COC(C(=O)O)P(=O)(NC(C)C)NC(C)C)C(O)C3O)nnc12] | 561.23 | 95.5 |
| 111 | 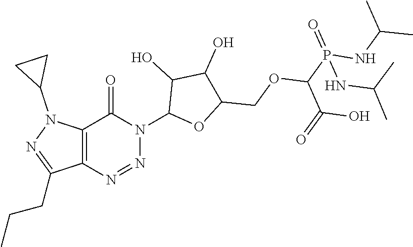 [SMILES: CCCc1nn(C2CC2)c2c(=O)n(C3OC(COC(C(=O)O)P(=O)(NC(C)C)NC(C)C)C(O)C3O)nnc12] | 571.25 | 96.2 |
| 112 | 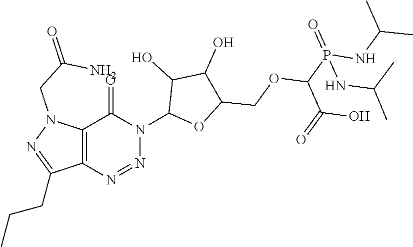 [SMILES: CCCc1nn(CC(N)=O)c2c(=O)n(C3OC(COC(C(=O)O)P(=O)(NC(C)C)NC(C)C)C(O)C3O)nnc12] | 588.24 | 95.8 |
| 113 | 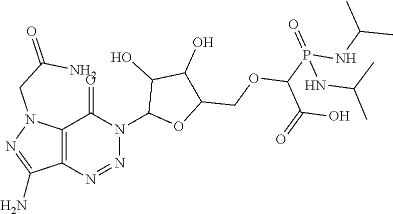 [SMILES: CC(C)NP(=O)(NC(C)C)C(OCC1OC(n2nnc3c(N)nn(CC(N)=O)c3c2=O)C(O)C1O)C(=O)O] | 561.21 | 96.9 |
| 114 | 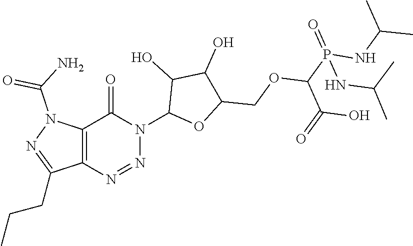 [SMILES: CCCc1nn(C(N)=O)c2c(=O)n(C3OC(COC(C(=O)O)P(=O)(NC(C)C)NC(C)C)C(O)C3O)nnc12] | 574.23 | 95.1 |
| 115 | 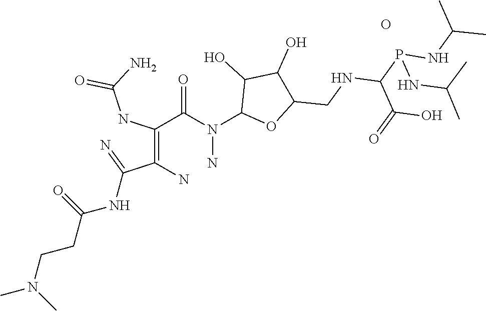 | 645.27 | 95.0 |
| 116 | 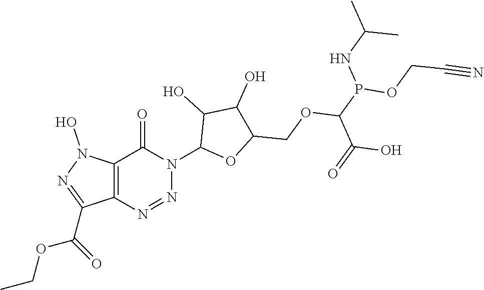 [SMILES: CCOC(=O)c1nn(O)c2c(=O)n(C3OC(COC(C(=O)O)P(NC(C)C)OCC#N)C(O)C3O)nnc12] | 559.14 | 98.6 |
| 117 | 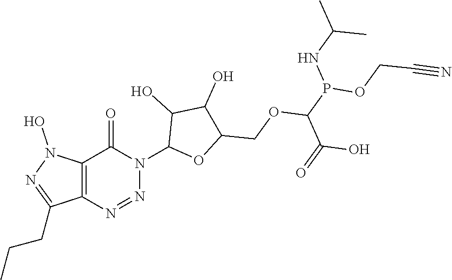 [SMILES: CCCc1nn(O)c2c(=O)n(C3OC(COC(C(=O)O)P(NC(C)C)OCC#N)C(O)C3O)nnc12] | 529.17 | 97.4 |
| 118 | 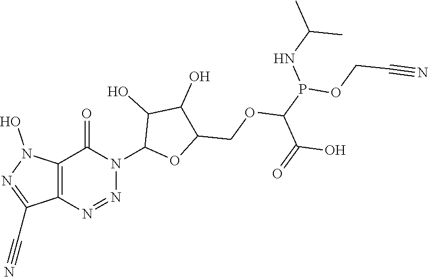 [SMILES: CC(C)NP(OCC#N)C(OCC1OC(n2nnc3c(C#N)nn(O)c3c2=O)C(O)C1O)C(=O)O] | 512.12 | 95.4 |
| 119 | 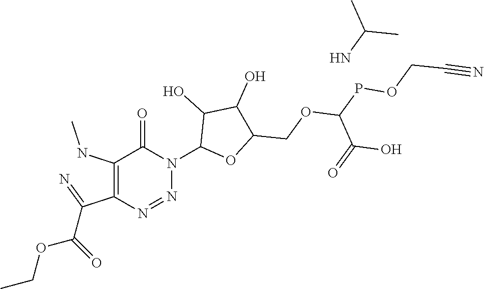 | 557.16 | 96.9 |
| 120 | 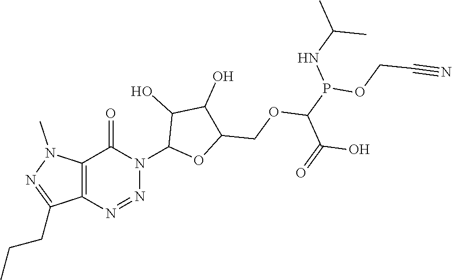 [SMILES: CCCc1nn(C)c2c(=O)n(C3OC(COC(C(=O)O)P(NC(C)C)OCC#N)C(O)C3O)nnc12] | 527.19 | 97.0 |
| 121 | 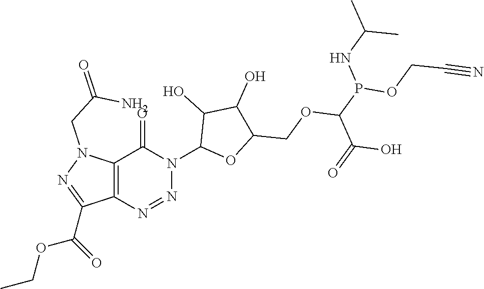 [SMILES: CCOC(=O)c1nn(CC(N)=O)c2c(=O)n(C3OC(COC(C(=O)O)P(NC(C)C)OCC#N)C(O)C3O)nnc12] | 600.17 | 95.0 |
| 122 | 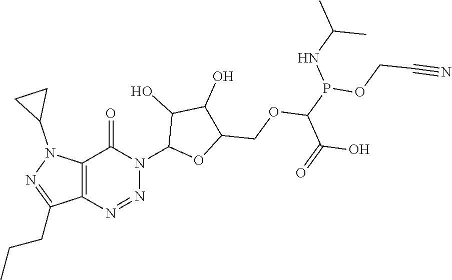 [SMILES: CCCc1nn(C2CC2)c2c(=O)n(C3OC(COC(C(=O)O)P(NC(C)C)OCC#N)C(O)C3O)nnc12] | 553.20 | 96.9 |
| 123 | 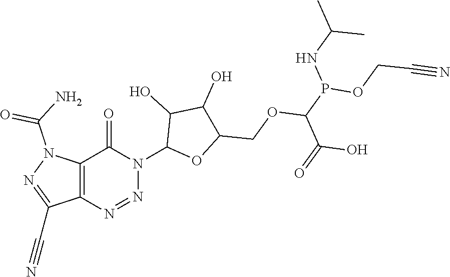 [SMILES: CC(C)NP(OCC#N)C(OCC1OC(n2nnc3c(C#N)nn(C(N)=O)c3c2=O)C(O)C1O)C(=O)O] | 539.13 | 95.6 |
| 124 | 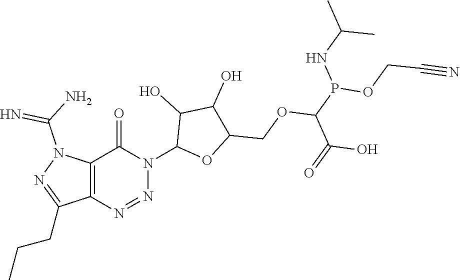 [SMILES: CCCc1nn(C(=N)N)c2c(=O)n(C3OC(COC(C(=O)O)P(NC(C)C)OCC#N)C(O)C3O)nnc12] | 555.20 | 96.0 |
| 125 | 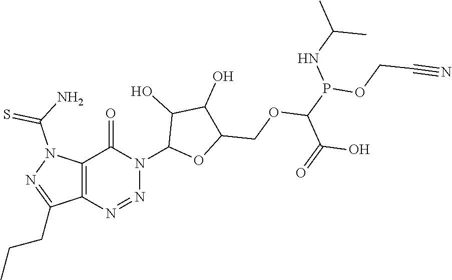 [SMILES: CCCc1nn(C(N)=S)c2c(=O)n(C3OC(COC(C(=O)O)P(NC(C)C)OCC#N)C(O)C3O)nnc12] | 572.16 | 95.9 |
| 126 | 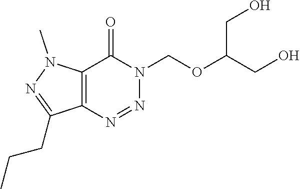 [SMILES: CCCc1nn(C)c2c(=O)n(COC(CO)CO)nnc12] | 297.14 | 98.3 |
| 127 | 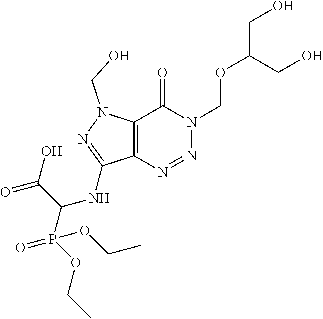 [SMILES: CCOP(=O)(OCC)C(Nc1nn(CO)c2c(=O)n(COC(CO)CO)nnc12)C(=O)O] | 480.14 | 96.0 |
| 128 | 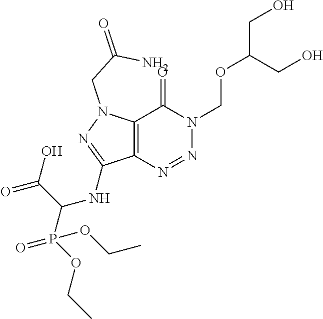 [SMILES: CCOP(=O)(OCC)C(Nc1nn(CC(N)=O)c2c(=O)n(COC(CO)CO)nnc12)C(=O)O] | 507.15 | 96.9 |
| 129 | 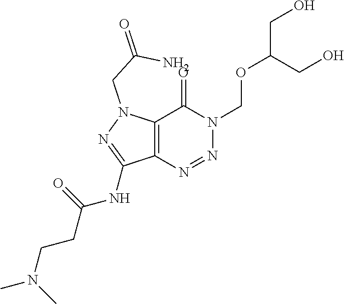 [SMILES: CN(C)CCC(=O)Nc1nn(CC(N)=O)c2c(=O)n(COC(CO)CO)nnc12] | 412.18 | 97.6 |
| 130 | 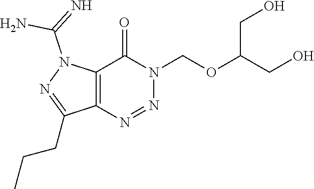 [SMILES: CCCc1nn(C(=N)N)c2c(=O)n(COC(CO)CO)nnc12] | 325.15 | 95.5 |
| 131 | 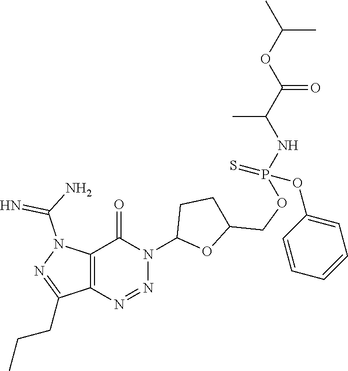 [SMILES: CCCc1nn(C(=N)N)c2c(=O)n(C3CCC(COP(=S)(NC(C)C(=O)OC(C)C)Oc4ccccc4)O3)nnc12] | 606.21 | 95.0 |
| 132 | 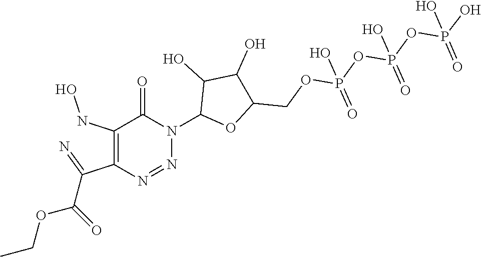 [SMILES: CCOC(=O)C(=N)c1nnn(C2OC(COP(=O)(O)OP(=O)(O)OP(=O)(O)O)C(O)C2O)c(=O)c1NO] | 596.99 | 96.6 |
| 133 | 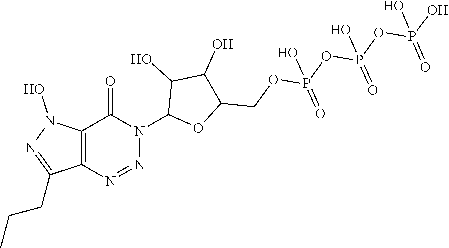 [SMILES: CCCc1nn(O)c2c(=O)n(C3OC(COP(=O)(O)OP(=O)(O)OP(=O)(O)O)C(O)C3O)nnc12] | 567.02 | 95.6 |
| 134 | 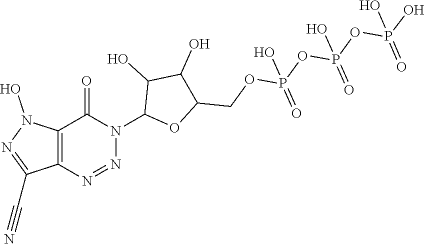 [SMILES: N#Cc1nn(O)c2c(=O)n(C3OC(COP(=O)(O)OP(=O)(O)OP(=O)(O)O)C(O)C3O)nnc12] | 549.97 | 95.2 |
| 135 | 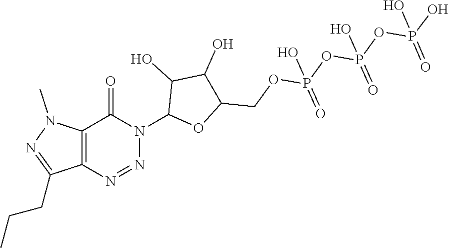 [SMILES: CCCc1nn(C)c2c(=O)n(C3OC(COP(=O)(O)OP(=O)(O)OP(=O)(O)O)C(O)C3O)nnc12] | 565.04 | 99.1 |
| 136 | 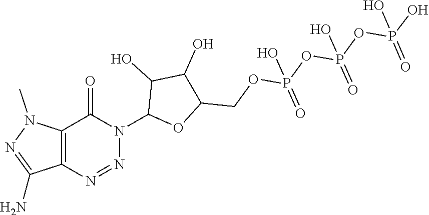 [SMILES: Cn1nc(N)c2nnn(C3OC(COP(=O)(O)OP(=O)(O)OP(=O)(O)O)C(O)C3O)c(=O)c21] | 538.00 | 95.4 |
| 137 | 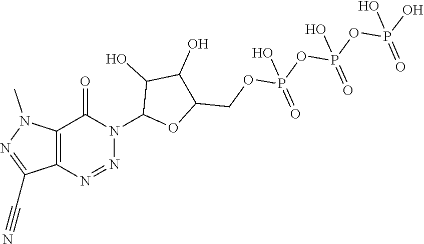 [SMILES: Cn1nc(C#N)c2nnn(C3OC(COP(=O)(O)OP(=O)(O)OP(=O)(O)O)C(O)C3O)c(=O)c21] | 547.99 | 95.1 |
| 138 | 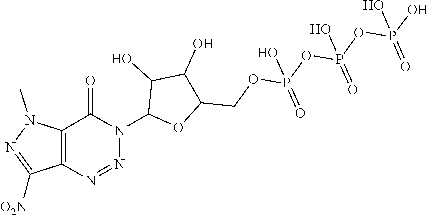 [SMILES: Cn1nc([N+](=O)[O-])c2nnn(C3OC(COP(=O)(O)OP(=O)(O)OP(=O)(O)O)C(O)C3O)c(=O)c21] | 567.98 | 96.9 |
| 139 | 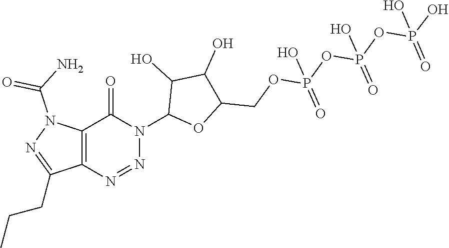 [SMILES: CCCc1nn(C(N)=O)c2c(=O)n(C3OC(COP(=O)(O)OP(=O)(O)OP(=O)(O)O)C(O)C3O)nnc12] | 594.03 | 96.6 |
| 140 | 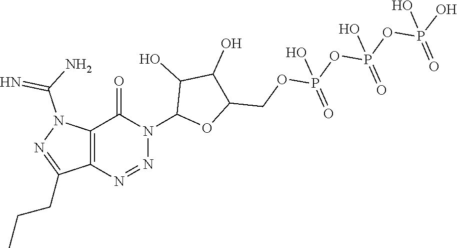 [SMILES: CCCc1nn(C(=N)N)c2c(=O)n(C3OC(COP(=O)(O)OP(=O)(O)OP(=O)(O)O)C(O)C3O)nnc12] | 593.04 | 97.9 |
| 141 | 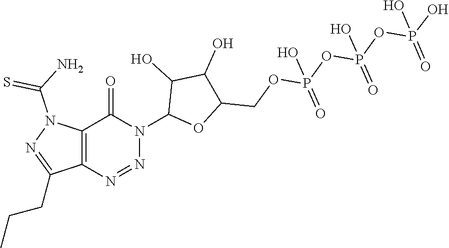 [SMILES: CCCc1nn(C(N)=S)c2c(=O)n(C3OC(COP(=O)(O)OP(=O)(O)OP(=O)(O)O)C(O)C3O)nnc12] | 610.00 | 95.2 |
| 142 | 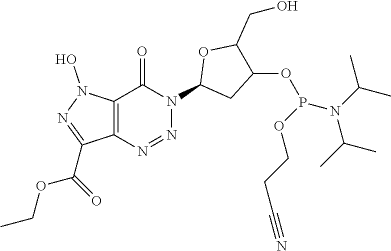 [SMILES: CCOC(=O)c1nn(O)c2c(=O)n([C@@H]3CC(OP(OCCC#N)N(C(C)C)C(C)C)C(CO)O3)nnc12] | 541.20 | 96.4 |
| 143 | 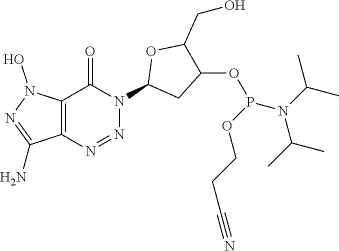 [SMILES: CC(C)N(C(C)C)P(OCCC#N)OC1C[C@@H](n2nnc3c(N)nn(O)c3c2=O)OC1CO] | 484.19 | 95.9 |
| 144 | 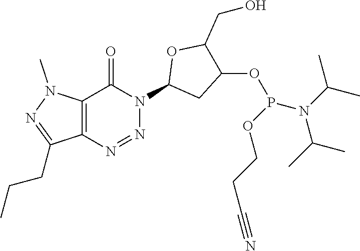 [SMILES: CCCc1nn(C)c2c(=O)n([C@@H]3CC(OP(OCCC#N)N(C(C)C)C(C)C)C(CO)O3)nnc12] | 509.25 | 96.9 |
| 145 | 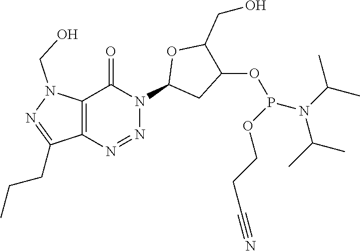 [SMILES: CCCc1nn(CO)c2c(=O)n([C@@H]3CC(OP(OCCC#N)N(C(C)C)C(C)C)C(CO)O3)nnc12] | 525.25 | 95.6 |
| 146 | 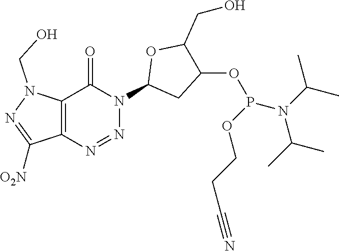 [SMILES: CC(C)N(C(C)C)P(OCCC#N)OC1C[C@@H](n2nnc3c([N+](=O)[O-])nn(CO)c3c2=O)OC1CO] | 528.18 | 95.1 |
| 147 | 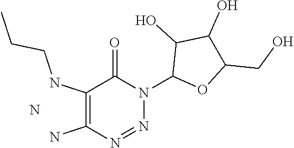 | 312.12 | 98.6 |
| 148 | 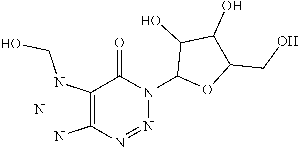 | 300.08 | 97.3 |
| 149 | 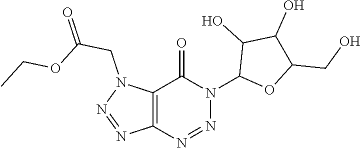 [SMILES: CCOC(=O)Cn1nnc2nnn(C3OC(CO)C(O)C3O)c(=O)c21] | 356.11 | 96.2 |
| 150 | 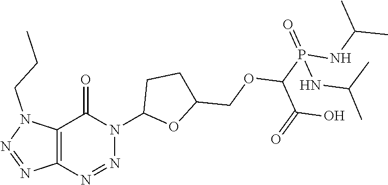 [SMILES: CCCn1nnc2nnn(C3CCC(COC(C(=O)O)P(=O)(NC(C)C)NC(C)C)O3)c(=O)c21] | 500.23 | 95.9 |
| 151 | 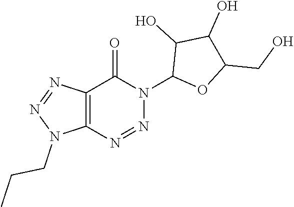 [SMILES: CCCn1nnc2c(=O)n(C3OC(CO)C(O)C3O)nnc21] | 312.12 | 97.0 |
| 152 | 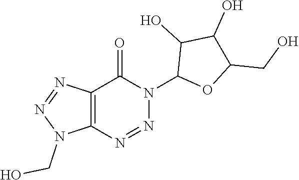 [SMILES: O=c1c2nnn(CO)c2nnn1C1OC(CO)C(O)C1O] | 300.08 | 96.8 |
| 153 | 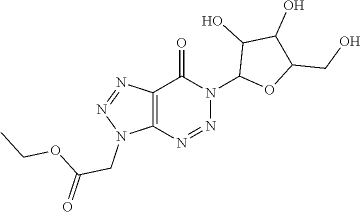 [SMILES: CCOC(=O)Cn1nnc2c(=O)n(C3OC(CO)C(O)C3O)nnc21] | 356.11 | 95.6 |
| 154 | 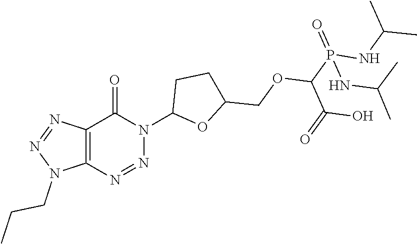 [SMILES: CCCn1nnc2c(=O)n(C3CCC(COC(C(=O)O)P(=O)(NC(C)C)NC(C)C)O3)nnc21] | 500.23 | 95.0 |
| 155 | 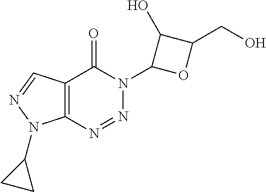 [SMILES: O=c1c2cnn(C3CC3)c2nnn1C1OC(CO)C1O] | 279.10 | 96.2 |
| 156 | 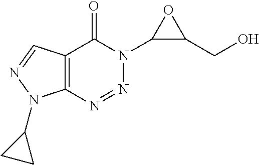 [SMILES: O=c1c2cnn(C3CC3)c2nnn1C1OC1CO] | 249.09 | 95.9 |
| 157 | 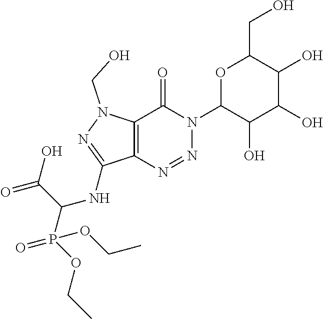 [SMILES: CCOP(=O)(OCC)C(Nc1nn(CO)c2c(=O)n(C3OC(CO)C(O)C(O)C3O)nnc12)C(=O)O] | 538.14 | 96.2 |
| 158 | 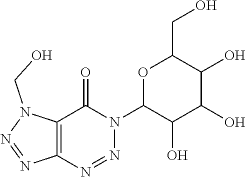 [SMILES: O=c1c2c(nnn1C1OC(CO)C(O)C(O)C1O)nnn2CO] | 330.09 | 96.5 |
| 159 | 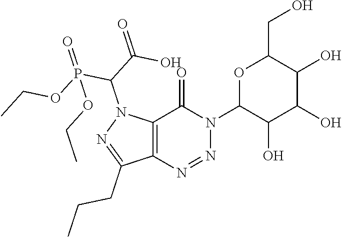 [SMILES: CCCc1nn(C(C(=O)O)P(=O)(OCC)OCC)c2c(=O)n(C3OC(CO)C(O)C(O)C3O)nnc12] | 535.17 | 95.0 |
| 160 | 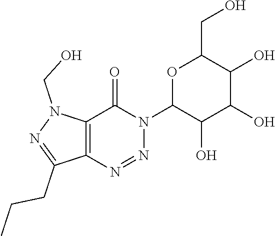 [SMILES: CCCc1nn(CO)c2c(=O)n(C3OC(CO)C(O)C(O)C3O)nnc12] | 371.14 | 96.1 |
| 161 | 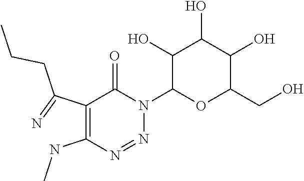 [SMILES: CCCC(=N)c1c(NC)nnn(C2OC(CO)C(O)C(O)C2O)c1=O] | 355.15 | 95.9 |
| 162 | 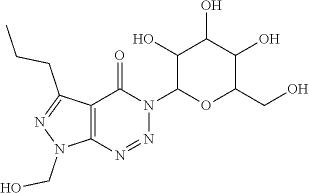 [SMILES: CCCc1nn(CO)c2nnn(C3OC(CO)C(O)C(O)C3O)c(=O)c12] | 371.14 | 96.2 |
| 163 | 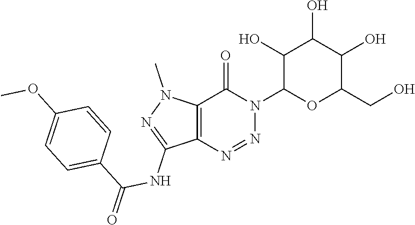 [SMILES: COc1ccc(C(=O)Nc2nn(C)c3c(=O)n(C4OC(CO)C(O)C(O)C4O)nnc23)cc1] | 462.15 | 96.9 |
| 164 | 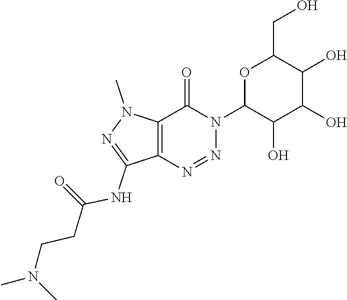 [SMILES: CN(C)CCC(=O)Nc1nn(C)c2c(=O)n(C3OC(CO)C(O)C(O)C3O)nnc12] | 427.18 | 95.7 |
| 165 | 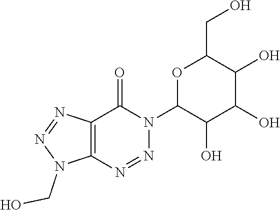 [SMILES: O=c1c2nnn(CO)c2nnn1C1OC(CO)C(O)C(O)C1O] | 330.09 | 95.8 |
| 166 | 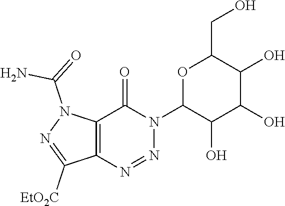 [SMILES: CCOC(=O)c1nn(C(N)=O)c2c(=O)n(C3OC(CO)C(O)C(O)C3O)nnc12] | 414.11 | 95.4 |
| 167 | 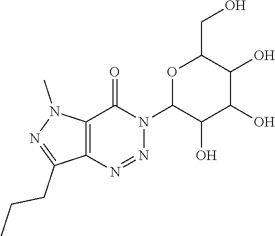 [SMILES: CCCc1nn(C)c2c(=O)n(C3OC(CO)C(O)C(O)C3O)nnc12] | 355.15 | 97.1 |

### General Method of Injection Preparation

### Example 168 Preparation of Injection I

The example compound 14 5.0 g, ethanol 600 mL (95%), 1, 2-propanediol 600 mL and Tween (80) 100 mL were dissolved and the injection water was added up to total volume of 5000 mL. The solution was filtered with 0.22 μm membrane filter and sterilized for 30 min at 100° C. to obtain 1000 preparation of injection 5 mg/5 mL.

### Example 169 Preparation of Injection II

The example compound 15 8.0 g, DMSO 50 mL, 1, 2-propanediol 100 mL and Tween 80 100 mL were dissolved and the injection water was added up to total volume of 5000 mL. The solution was filtered with 0.22 μm membrane filter and sterilized 30 min at 100° C. to obtain 1000 preparation of injection 8 mg/5 mL.

### Biological Activity

### Example 170 Efficacy Studies of Anticancer In Vitro

Cell lines: Human pancreatic cancer cell line Panc-1, human colorectal cancer cell line HT₂₉, human lung cancer cell line NCI-H₄₆₀ and human breast cancer cell line MCF7; the medium: s DMEM (Gibco BRL), containing 10% fetal calf serum (Gibco BRL) and 2 mM L-glutamine (Gibco BRL).

Test samples: example compounds 7, 15, 82, 89, 116 and 135. The samples were dissolved in dimethyl sulfoxide (DMSO, Sigma, United States) and medium was added to the final concentration of 0.5%. Cyclophosphamide ware as positive control (Aldrich, United States, purity>96%).

General Method: cells were digested with trypsin and dispersed into single cells in the medium containing penicillin (25 U/ml) and streptomycin (25 μg/mL). The cells were seeded in 96-well culture plates (Corning Incorporated), at 37° C., with 5% CO₂ present for 24 hours. The culture medium was removed, 1-100 μm test compounds were added, cultured for 48 hours. Culture medium was removed and thiophene Wow blue (MTT, USA Sigma products) was added. The result was assayed by SK601-based microplate reader (Japan Seikagaku company's products), 570 nm/630 nm optical density (OD). Cell viability determined by the formula: (Experimental group OD/control OD)×100%; CTX and the positive control subjects treated in the same material simultaneously.

### Results

Inhibition of colorectal cancer: as shown in table 2 six test compounds showed anti-proliferative effect on HT₂₉. Example compounds 15 showed close values to CTX of anti-proliferate on HT₂₉, IC₅₀ (the half maximal inhibitory concentration) values, and 95% confidence limit of 3.92 (1.99-5.46) μg/mL (P<0.05), CTX of the IC₅₀ 95% confidence limit of 2.26 (2.08-4.89) μg/mL (P<0.05); anti-proliferative effect of compound 7, 89, 82 and 135 is relatively weak, IC₅₀ and 95% confidence limits were 18.81 (15.7-21.38) μg/mL, 13.13 (9.54-15.31) μg/mL, 10.21 (8.01-11.74) μg/mL (P<0.05) and 17.96 (14.32-22.03) μg/mL, anti-proliferative effect of compound 116 is the strongest, IC₅₀ and 95% confidence limit of 2.68 (1.32-4.09) μg/mL (P<0.05) by twice experiment with good reproducibility.

Inhibition of pancreatic cancer: as shown in table 2 seven test compounds showed anti-proliferative effect on Panc-1. Example compound 15 and 116 showed anti-proliferative effect on Panc-1 at IC₅₀ (the half maximal inhibitory concentration) values 2.68 (1.25-3.51) μg/mL and 2.19 (0.89-3.45) μg/mL, IC₅₀ (P<0.05), compound 7, 82, 89 and 135 of Panc-1 half inhibitory concentration (IC₅₀) and 95% confidence limits were 51.32 (48.95-55.51) μg/mL (P<0.05), 52.97 (50.20-54.41) μg/mL, 10.01 (7.24-12.91) μg/mL (P<0.05) and 53.45 (45.32-58.67) μg/mL (P<0.05). Compound 116 a strong anti-proliferative effect, better than CTX IC₅₀ and 95% confidence limit of 2.23 (1.81-2.46) μg/mL by twice experiment with good reproducibility.

Inhibitory effect on lung cancer NCI-H460 cells: as shown in table 2. Positive control IC₅₀ of CTX and 95% confidence limit of 4.30 (3.04-5.13) μg/mL (P<0.05). Test compound 7 and 89 of the IC₅₀ and 95% confidence limits were 5.18 (4.42-7.03) (P<0.05) and 8.52 (6.66-9.14) (P<0.05). IC₅₀ value of test compound 15, 82, 116 and 135 are 22.01 (20.42-25.03) (P<0.05), 60.52 (58.66-62.14) (P<0.05), 4.62 (3.23-6.12) (P<0.05) and 14.01 (12.25-15.77) with 95% confidence limits. Compound 116 and compound 7 is more sensitive to NCI-H460 cells, by twice experiment with good reproducibility.

Inhibitory effect on breast cancer MCF7 cells: as shown in table 2, test compound 7, 15, 82, 89, 116 and 135 of the IC₅₀ were respectively 6.18 (3.42-8.03), 2.73 (1.21-4.02), 34.33 (30.54-40.78), 67.45 (58.89-79.51), 12.94 (9.78-15.13) and 5.70 (4.22-6.96) with 95% confidence limits. Positive control of IC₅₀ of CTX and 95% confidence limit of 0.92 (0.03-2.02) μg/mL. Compound 15 is more sensitive to NCI-H460 cells, IC₅₀ (P<0.05).

In this experiment twice, good reproducibility. Inhibitory effect on colorectal cancer cell lines HT-29, pancreatic cancer Panc-1, lung cancer NCI-H460 and breast MCF7 of test compound 7, 15, 82, 89 and 116 with cyclophosphamide as a control experiment showed that all compounds are sensitive to colorectal cancer, pancreatic cancer and breast cancer cells. Compound 15 and 116 showed higher inhibition activity on colorectal cancer and pancreatic cancer.

TABLE 2
Six compounds IC₅₀ (μM)

| | Colorectal | Pancreatic | Lung | Breast |
|---|---|---|---|---|
| | cancer | cancer | cancer | cancer |
| IC₅₀ | HT-29 | Panc-1 | NCI-H460 | MCF7 |
| Compound 7 | 18.81 (15.7-21.38) | 51.32 (48.95-55.51) | 5.18 (4.42-7.03) | 6.18 (3.42-8.03) |
| Compound 15 | 3.92 (1.99-5.46) | 2.68 (1.25-3.51) | 22.01 (20.42-25.03) | 2.73 (1.21-4.02) |
| Compound 82 | 13.13 (9.54-15.31) | 52.97 (50.20-54.41) | 60.52 (58.66-62.14) | 34.33 (30.54-40.78) |
| Compound 89 | 10.21 (8.01-11.74) | 10.01 (7.24-12.91) | 8.52 (6.66-9.14) | 67.45 (58.89-79.51) |
| Compound 116 | 2.68 (1.32-4.09) | 2.19 (0.89-3.45) | 4.62 (3.23-6.12) | 12.94 (9.78-15.13) |
| Compound 135 | 17.96 (14.32-22.03) | 53.45 (45.32-58.67) | 14.01 (12.25-15.77) | 5.70 (4.22-6.96) |
| CTX | 2.26 (2.08-4.89) | 2.23 (1.81-2.46) | 4.30 (3.04-5.13) | 0.92 (0.03-2.02 |

### Example 171 Efficacy Studies of Anticancer In Vivo

Test samples: Examples of 14, 15, 16, 17, 18, 20, 22, 23, 24, 25, 26, 27, 28, 29, 30, 31, 32, 36, 40, 48, 49, 50, 54, 55, 56, 58, 59, 60, 61, 68, 70, 72, 73, 74, 76, 77, 79, 80, 81, 82, 83, 85, 86, 88, 89, 90, 91, 94, 95, 97, 98, 99, 100, 103, 106, 116, 117, 120, 126, 129, 135, 140, 147, 148, 151, 152 and 154 (see Table 1).

Experimental animals: Kunming healthy mice provided by the Animal Center of Beijing Academy of Military Medical.

Tumor strains: mice sarcoma S₁₈₀ for ascites passaged from Beijing Academy of Military Medical Institute of Pharmacology.

General Method: Xenografts cultured S₁₈₀ tumor cells were implanted subcutaneously into the flank region of mice and tumors were allowed to grow to the desired average size of 100 mg. The mice were randomized into control and treatment groups with 10 mice per group. The control group was injected with the vehicle used to dissolve the drug. Other groups received the test compounds (example compound) and positive group, Cyclophosphamide (CTX) and Paclitaxel (Taxol) at the dose and schedule as indicated in Table VI. Injections were I.V. via the tail vein. Tumor measurements were taken every other day 20% tumor growth inhibition which was not statistically significant.

Results: The in-vitro anticancer screening result sees Table 3

TABLE 3
Growth Inhibition of S₁₈₀ sarcoma ( X̅ ± SD, n = 16)

| Example | | | Inhibition rate |
|---|---|---|---|
| Compound | Administrtion | mg/kg | (%) |
| Saline | | | − |
| CTX | iv | 15 | ++ |
| Taxol | ip | 20 | ++ |
| 14 | iv | 12.5 | ++ |
| 15 | iv | 50 | ++ |
| 16 | ip | 100 | + |
| 17 | iv | 25 | ++ |
| 18 | ip | 200 | + |
| 20 | ip | 200 | + |
| 22 | ip | 200 | ++ |
| 23 | ip | 100 | ++ |
| 24 | ip | 300 | ++ |
| 25 | ip | 200 | ++ |
| 26 | ip | 70 | ++ |
| 27 | iv | 15 | + |
| 28 | iv | 20 | ++ |
| 29 | iv | 15 | + |
| 30 | ip | 400 | ++ |
| 31 | ip | 100 | ++ |
| 32 | iv | 30 | ++ |
| 36 | ip | 200 | ++ |
| 40 | iv | 50 | + |
| 48 | iv | 50 | ++ |
| 49 | ip | 100 | ++ |
| 50 | iv | 15 | ++ |
| 54 | ip | 150 | ++ |
| 55 | ip | 100 | ++ |
| 56 | ip | 100 | ++ |
| 58 | ip | 100 | ++ |
| 59 | iv | 50 | + |
| 60 | iv | 15 | ++ |
| 61 | iv | 12.5 | + |
| 68 | iv | 20 | ++ |
| 70 | ip | 100 | ++ |
| 72 | iv | 25 | ++ |
| 73 | ip | 200 | ++ |
| 74 | ip | 200 | ++ |
| 76 | ip | 400 | ++ |
| 77 | ip | 100 | ++ |
| 79 | ip | 300 | + |
| 80 | ip | 200 | + |
| 81 | ip | 100 | ++ |
| 82 | iv | 15 | + |
| 83 | iv | 20 | + |
| 85 | ip | 400 | + |
| 86 | ip | 200 | + |
| 88 | ip | 400 | ++ |
| 89 | ip | 100 | ++ |
| 90 | iv | 30 | + |
| 91 | ip | 200 | ++ |
| 94 | iv | 30 | ++ |
| 95 | iv | 50 | ++ |
| 97 | ip | 100 | ++ |
| 98 | ip | 70 | + |
| 99 | ip | 150 | + |
| 100 | ip | 100 | ++ |
| 103 | ip | 100 | + |
| 106 | iv | 50 | + |
| 116 | iv | 50 | ++ |
| 117 | iv | 15 | ++ |
| 120 | iv | 12.5 | ++ |
| 126 | iv | 50 | + |
| 129 | ip | 100 | + |
| 135 | iv | 25 | ++ |
| 140 | ip | 200 | + |
| 147 | ip | 300 | + |
| 148 | ip | 200 | + |
| 151 | ip | 200 | + |
| 152 | ip | 400 | + |
| 154 | ip | 100 | + |

Note
All vehicle: DMSO; dose range: 4-100 mg/kg;
− represent of Inhibition rate % <10,
+ represent of Inhibition rate % <20,
++ represent of Inhibition rate % <40, Inhibition rate more than 40% of the sample was statistically significant better than control group.

### CONCLUSION

Table 3. showed the in-vivo experimental data of anti-tumor efficacy. It is especially noteworthy that the example compounds of 14, 15, 17, 22, 23, 24, 25, 26, 28, 30, 31, 32, 36, 48, 49, 50, 54, 55, 56, 58, 60, 68, 70, 72, 73, 74, 76, 77, 81, 88, 89, 91, 94, 95, 97, 100, 116, 117, 120 and 135 are statistically significant.

1. A compound of the formula I: 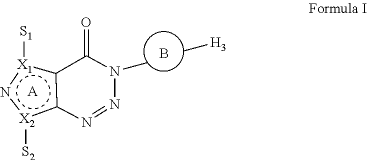
or stereoisomers, tautoers, prodrugs, pharmaceutically acceptable salts, complex salts or solvates thereof, wherein: the dotted lines [symbol] are absent or selected but is not limited from: —C—C—, —C═C—, —C-heterobond, —C=heterobond, 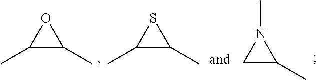
X₁ and X₂ is absent or independently selected but is not limited from: hydrogen, —C—, —S—, —O—, —NH—,
S₁ and S₂ is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, aryl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —C(O)OC₁₋₆alkyl, —C(O)NHC₁₋₆alkyl, P(O)(OH)₂, P(O)(O₁₋₆alkyl)₂, —(CH₂)₁₋₆P(O)(OH)₂, —(CH₂)₁₋₆P(O)(OC₁₋₆alkyl)₂, C₃₋₈amino acid, C₃₋₁₀(OH)₀₋₁₀polyhydroxide;
H₃ is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —(CH₂)₁₋₁₀(OH)₁₋₇, —CH((CH₂)₁₋₁₀(OH)₁₋₇)₁₋₃, —(CH₂)₁₋₁₀(OC(O)C₁₋₆alkyl)₁₋₇, —C(O)NHC₁₋₆alkyl, —P(O)(O₁₋₆alkyl)₂, —(CH₂)₀₋₆P(O)(OH)₂, —(CH₂)₀₋₆P(O)(OC₁₋₆alkyl)₂, —(CH₂)₀₋₆(P(O)(NC₁₋₆alkyl)₂)₁₋₃(OC₁₋₆alkyl)₂)₁₋₃, C₃₋₈amino acid.
A ring of 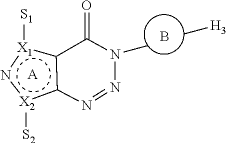
is selected but is not limited from: 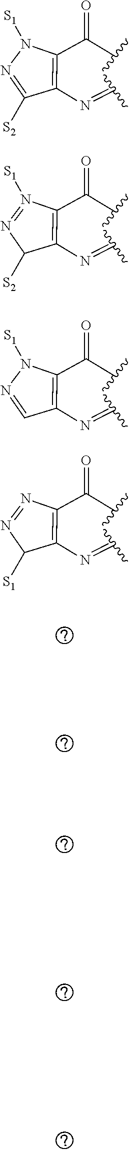
to form 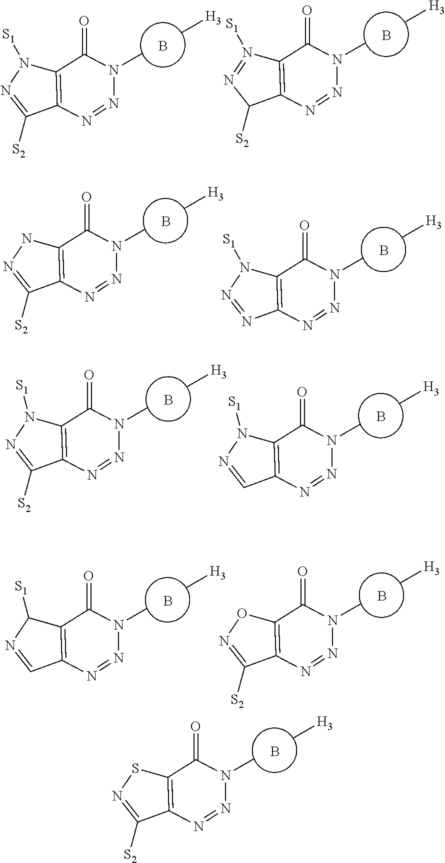
B ring of 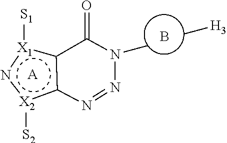
is absent or selected but is not limited from: 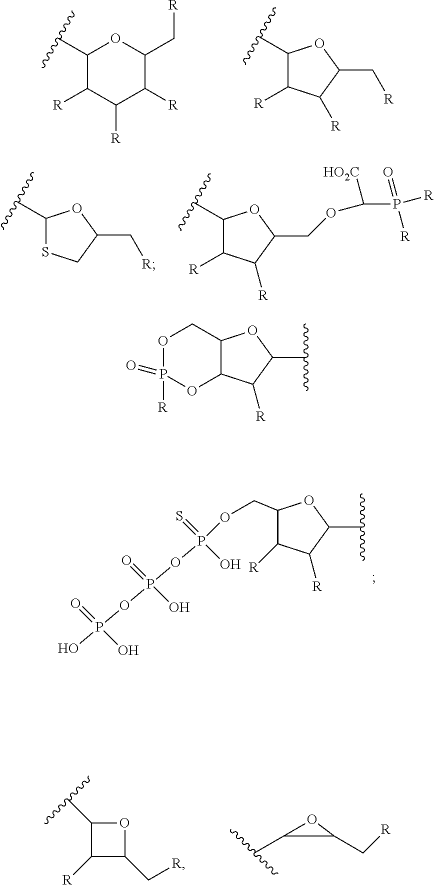 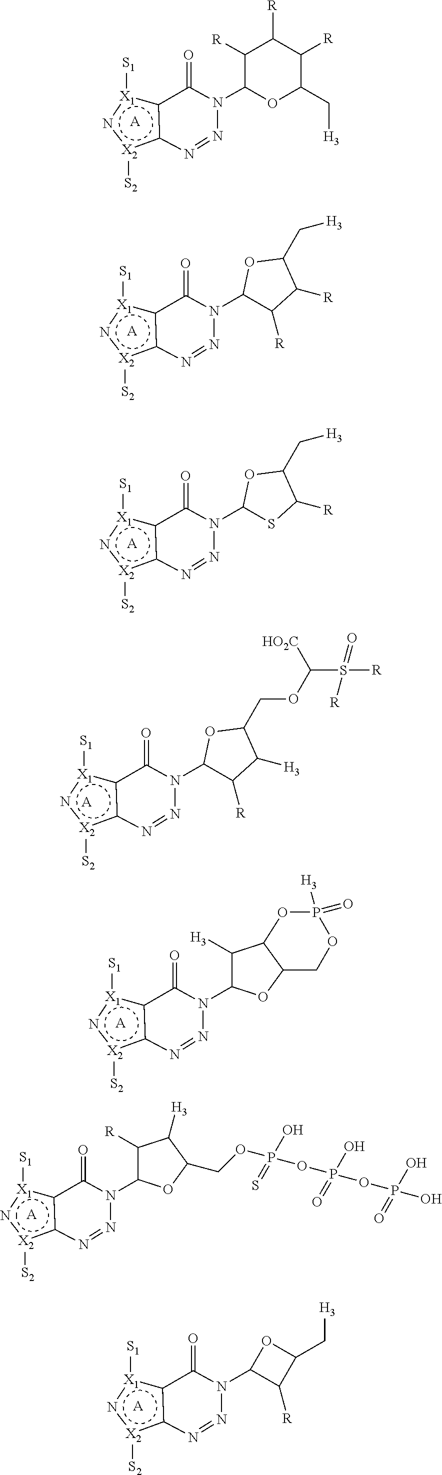 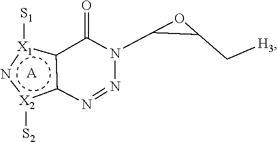
said R is selected but are not limited from hydrogen, halogen, —CN, —CF₃, —OH, —NH₂, guanidine, amidine, —SH, —P(O)(OH)₂, C₃₋₈amino acid, —OC₁₋₆alkyl, —OC₂₋₆alkenyl, —OC₂₋₆alkynyl, —OC₃₋₁₀cycloalkyl, —OC₃₋₁₀cycloalkenyl, O-aryl, —OC₃₋₁₀heterocycle, —OC₃₋₁₀heteroaryl, —NHC₁₋₆alkyl, —NHC₂₋₆alkenyl, —NHC₂₋₆alkynyl, —NHC₃₋₁₀cycloalkyl, —NHC₃₋₁₀cycloalkenyl, NH-aryl, —NHC₃₋₁₀heterocycle, —NH₃₋₁₀heteroaryl, —N(C₁₋₆alkyl)₂, —N(C₂₋₆alkenyl)₂, —N(C₂₋₆alkynyl)₂, —N(C₃₋₁₀cycloalkyl)₂, —N(C₃₋₁₀cycloalkenyl)₂, N(aryl)₂, —N(C₃₋₁₀heterocycle)₂, —N(C₃₋₁₀heteroaryl)₂, —NHSO₂C₁₋₆alkyl, —NHC(O)C₁₋₆alkyl, —SC₁₋₆alkyl, —SC₂₋₆alkenyl, —SC₂₋₆alkynyl, —SC₃₋₁₀cycloalkyl, —SC₃₋₁₀cycloalkenyl, S-aryl, —SC₃₋₁₀heterocycle, —SC₃₋₁₀heteroaryl, —SOC₁₋₆alkyl, —SO₂C₁₋₆alkyl, —SO₂NHC₁₋₆alkyl, —C₁₋₆alkyl, —C₂₋₆alkenyl, —C₂₋₆alkynyl, —C₃₋₁₀cycloalkyl, —C₃₋₁₀cycloalkenyl, -aryl, —C₃₋₁₀heterocycle, —C₃₋₁₀heteroaryl, —C(O)OH, —C(O)OC₁₋₆alkyl, —C(O)NHC₁₋₆alkyl, —OP(OC₁₋₆alkyl)₂, —OP(OC₂₋₆alkenyl)₂, —OP(OC₂₋₆alkynyl)₂, —OP(OC₃₋₁₀cycloalkyl)₂, —OP(OC₃₋₁₀cycloalkenyl)₂, OP-(Oaryl)₂, —OP(OC₃₋₁₀heterocycle)₂, —OP(OC₃₋₁₀heteroaryl)₂, —NP(O)(NC₁₋₆alkyl)(OC₁₋₆alkyl), —NP(O)(NC₂₋₆alkenyl)(OC₂₋₆alkenyl), —NP(O)(NC₂₋₆alkynyl)(OC₂₋₆alkynyl), —NP(O)(NC₃₋₁₀cycloalkyl)O (OC₃₋₁₀cycloalkyl), —NP(O)(NC₃₋₁₀cycloalkenyl)(OC₃₋₁₀cycloalkenyl), —NP(O)(N-aryl)(O-aryl), —NP(O)(NC₃₋₁₀heterocycle)(OC₃₋₁₀heterocycle), —NP(O)(NC₃₋₁₀heteroaryl)(OC₃₋₁₀heteroaryl), —NP(O)(NC₁₋₆alkyl)₂, —NP(O)(NC₂₋₆alkenyl)₂, —NP(O)(NC₂₋₆alkynyl)₂, —NP(O)(NC₃₋₁₀cycloalkyl)₂, —NP(O)(NC₃₋₁₀cycloalkenyl)2, —NP(O)(N-aryl)₂, —NP(O)(NC₃₋₁₀heterocycle)₂, —NP(O)(NC₃₋₁₀heteroaryl)₂.

2. According to the claim 1, wherein: a compound of novel triazinone nucleoside analogs is selected but is not limited from the exemplified examples or stereoisomers, tautomers, pharmaceutically acceptable salts, inorganic acid salt, organic acid salt, inorganic basic salt, organic basic salt, complex salt, prodrug or solvates thereof in association with a pharmaceutically acceptable excipient or carrier; in addition, an acid or a base may be incorporated into the composition to facilitate processing, to enhance stability, or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane and the like; bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, parabromophenylsulfonic acid, propionic acid, p-toluenesulfonic acid, salicylic acid, stearic acid, succinic acid, taimic acid, tartaric acid, thioglycolic acid, toluenesulfonic acid, uric acid, and the like.

3. A compound according to the claim 1, wherein: a process for the manufacture of a compound of formula I to form novel triazinone nucleoside analogs is obtained by modification of dicyclic triazinone compounds at 3-position with a bond of C—N, under catalysis at −78° C. to 90° C., by a solvent selected from THF, 1, 4-dioxane, N, N-dimethylformamide, N, N-dimethylacetamide, toluene, CH₃CN, CH₂Cl₂, C₆H₆ and a catalyst selected from SnCl₄, NH₃, SnCl₂, SiF₄ Bu₄NF, Hg(CN)₂, HgBr₂, Bu₄NI, Hg(OAc)₂, organic base, inorganic base, molecular sieves or alumina; in addition, the present invention is composed of the base to increases the solubility, to enhance the stability or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl) aminomethane.

4. According to claim 1, wherein: a compound of novel triazinone nucleoside analogs is independently at each occurrence, selected but is not limited from the example 1 to 167 and below list: diheterocyclic triazinone nucleosides.

5. A General Method according to the claim 1, wherein: the amount of one or more of the compounds of general formula I, or a pharmaceutically acceptable salt thereof, present in the composition for treating, preventing or slowing the progression of cancer, virus and other diseases, including inflammation, inflammatory diseases and immune system disease associated with cancer, alone or with the following drugs known to be used in conjunction dose in a range of about 0.001 mg-2.0 g/kg; means of various General Method s of treatment and therapy, where the cancers are selected but are not limited from the group consisting of busulfan, cisplatin, mitomycin C, carboplatin, colchichine, vinblastine, paclitaxel, docetaxel, camptochecin, topotecan, doxorubicin, etoposide, 5-azacytidine, 5-fluorouracil, methotrexate, 5-fluoro-2′-deoxy-uridine, ara-C, hydroxylurea, thioguanine, melphalan, chlorambucil, cyclo phosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin tamsulosin, CB-64D, CB-184, haloperidol, lovastatin, simvastatin, pravastatin, fluvastatin, atorvastatin, cerivastatin, amprenavir, abavavir, indinavir, nelfinavir, tipranavir, ritonavir, saquinavir, bexarotene, tretinoin, 13-cis-retinoic acid, 9-cis-retinoic acid, difluoromethylornithine, fenretinide, N-4-carboxyphenyl retinamide, lactacystin, genistein, flavopiridol, roscovitine, olomoucine, celecoxib, valecoxib, rofecoxib and alanosine, CGP-73547, CGP-61755, DMP-450, ABT-378, AG1776, BMS232,632, ILX23-7553, MG-132, PS341, Gleevec®, ZD1839, SH268, CEP2563, SU6668, SU11248, EMD121974, R115777, SCH66336, L-778,123, BAL9611, TAN-1813 or/and UCN-01.

6. A general method according to the claim 1, wherein: a compound of novel triazinone nucleoside analogs is selected for treating, preventing or slowing the progression of the group consisting of Hodgkin's disease, non-Hodgkin's, lymphoma, acute and chronic lymphocytic leukemias, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogentic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortical carcinoma, skin cancer and prostatic carcinoma.

7. A compound according to the claim 1, wherein: the administration of a compound of diheterocyclic triazinone nucleoside derivatives and analogs may be by oral route, parenteral, subcutaneous, intravenous, intramuscular, intra-peritoneal, transdermal, buccal, intrathecal, intracranial, intranasal or topical routes.

8. A compound according to the claim 1, wherein: a compound of diheterocyclic triazinone nucleoside derivatives and analogs is, independently at each occurrence, selected but is not limited from the example 1 to 167 and below list:
2-(diethoxyphosphoryl)-2-((5-hydroxy-3-(3-(hydroxymethyl)oxiran-2-yl)-4-oxo-4,5-dihydro-3H-pyrazolo[4,3-d][1,2,3]triazin-7-yl)amino)acetic acid, ethyl 7-hydroxy-2-(3-(hydroxymethyl)oxiran-2-yl)-1-oxo-2,7-dihydro-H-pyrazolo[4,3-d][1,2,3]triazine-5-carboxylate, N-(5-hydroxy-3-(3-(hydroxymethyl)oxiran-2-yl)-4-oxo-4,5-dihydro-3H-pyrazolo[4,3-d][1,2,3]triazin-7-yl)-4-methoxybenzamide, 3-(dimethylamino)-N-(5-hydroxy-3-(3-(hydroxymethyl)oxiran-2-yl)-4-oxo-4,5-dihydro-3H-pyrazolo[4,3-d][1,2,3]triazin-7-yl)propanamide, 7-hydroxy-2-(3-(hydroxy methyl)oxiran-2-yl)-5-(piperidin-1-yl)-2,7-dihydro-1H-pyrazolo[4,3-d][1,2,3]triazin-1-one, 7-hydroxy-2-(3-(hydroxymethyl)oxiran-2-yl)-5-nitro-2,7-dihydro-H-pyrazolo[4,3-d][1,2,3]triazin-1-one, ethyl 7-(hydroxymethyl)-2-(3-(hydroxymethyl)oxiran-2-yl)-1-oxo-2,7-dihydro-1H-pyrazolo[4, 3-d][1,2,3]triazine-5-carboxylate, ethyl 7-(2-amino-2-oxoethyl)-2-(3-(hydroxymethyl)oxiran-2-yl)-1-oxo-2,7-dihydro-1H-pyrazolo[4,3-d][1,2,3]triazine-5-carboxylate, ethyl 7-cyclopropyl-2-(3-(hydroxymethyl)oxiran-2-yl)-1-oxo-2,7-dihydro-H-pyrazolo[4,3-d][1,2,3]triazine-5-carboxylate, ethyl 7-carbamoyl-2-(3-(hydroxymethyl)oxiran-2-yl)-1-oxo-2,7-dihydro-H-pyrazolo[4,3-d][1,2,3]triazine-5-carboxylate, 2-(diethoxyphosphoryl)-2-(7-(ethoxycarbonyl)-3-(3-(hydroxymethyl)oxiran-2-yl)-4-oxo-3H-pyrazolo[4,3-d][1,2,3]triazin-5(4H)-yl)acetic acid, 2-(diethoxyphosphoryl)-2-((3-(3,4-dihydroxy-5-(hydroxymethyl)tetrahydrofuran-2-yl)-5-hydroxy-4-oxo-4,5-dihydro-3H-pyrazolo[4,3-d][1,2,3]triazin-7-yl)amino) acetic acid, ethyl 3-(3,4-dihydroxy-5-(hydroxymethyl)tetrahydrofuran-2-yl)-5-hydroxy-4-oxo-4,5-dihydro-3H-pyrazolo[4,3-d][1,2,3]triazine-7-carboxylate, 2-(3,4-dihydroxy-5-(hydroxymethyl)tetrahydrofuran-2-yl)-7-hydroxy-5-propyl-2,7-dihydro-H-pyrazolo[4,3-d][1,2,3]triazin-1-one, 7-amino-3-(3,4-dihydroxy-5-(hydroxymethyl)tetrahydrofuran-2-yl)-5-hydroxy-3H-pyrazolo[4,3-d][1,2,3]triazin-4(5H)-one, N-(3-(3,4-dihydroxy-5-(hydroxymethyl)tetrahydrofuran-2-yl)-5-hydroxy-4-oxo-4,5-dihydro-3H-pyrazolo[4,3-d][1,2,3]triazin-7-yl)-3-(dimethylamino)propanamide, 3-(3,4-dihydroxy-5-(hydroxymethyl)tetrahydrofuran-2-yl)-5-hydroxy-4-oxo-4,5-dihydro-3H-pyrazolo[4,3-d][1,2,3]triazine-7-carbonitrile, 3-(3,4-dihydroxy-5-(hydroxymethyl)tetrahydrofuran-2-yl)-5-hydroxy-7-(piperidin-1-yl)-3H-pyrazolo[4,3-d][1,2,3]triazin-4(5H)-one, 3-(3,4-dihydroxy-5-(hydroxymethyl)tetrahydrofuran-2-yl)-5-hydroxy-7-nitro-3H-pyrazolo[4,3-d][1,2,3]triazin-4(5H)-one, N-(3-(3,4-dihydroxy-5-(hydroxymethyl) tetrahydrofuran-2-yl)-5-hydroxy-4-oxo-4,5-dihydro-3H-pyrazolo[4,3-d][1,2,3]triazin-7-yl)-4-methoxybenzamide, 2-(diethoxyphosphoryl)-2-((3-(3,4-dihydroxy-5-(hydroxymethyl)tetrahydrofuran-2-yl)-5-methyl-4-oxo-4,5-dihydro-3H-pyrazolo[4,3-d][1,2,3]triazin-7-yl)amino)acetic acid, ethyl 3-(3,4-dihydroxy-5-(hydroxymethyl)tetrahydrofuran-2-yl)-5-methyl-4-oxo-4,5-dihydro-3H-pyrazolo[4,3-d][1,2,3]triazine-7-carboxylate, 3-(3,4-dihydroxy-5-(hydroxymethyl)tetrahydrofuran-2-yl)-5-methyl-7-propyl-3H-pyrazolo[4,3-d][1,2,3]triazin-4(5H)-one, 7-amino-3-(3,4-dihydroxy-5-(hydroxymethyl)tetrahydrofuran-2-yl)-5-methyl-3H-pyrazolo[4,3-d][1,2,3]triazin-4(5H)-one, N-(3-(3,4-dihydroxy-5-(hydroxymethyl) tetrahydrofuran-2-yl)-5-methyl-4-oxo-4,5-dihydro-3H-pyrazolo[4,3-d][1,2,3]triazin-7-yl)-3-(dimethylamino)propanamide, 3-(3,4-dihydroxy-5-(hydroxymethyl)tetrahydrofuran-2-yl)-5-methyl-4-oxo-4,5-dihydro-3H-pyrazolo[4,3-d][1,2,3]triazine-7-carbonitrile, 3-(3,4-dihydroxy-5-(hydroxymethyl) tetrahydrofuran-2-yl)-5-methyl-7-(piperidin-1-yl)-3H-pyrazolo[4,3-d][1,2,3]triazin-4(5H)-one, 3-(3,4-dihydroxy-5-(hydroxymethyl)tetrahydrofuran-2-yl)-5-methyl-7-nitro-3H-pyrazolo[4,3-d][1,2,3]triazin-4 (5H)-one, N-(3-(3,4-dihydroxy-5-(hydroxymethyl)tetrahydrofuran-2-yl)-5-methyl-4-oxo-4,5-dihydro-3H-pyrazolo[4,3-d][1,2,3]triazin-7-yl)-4-methoxybenzamide, ethyl 3-(3,4-dihydroxy-5-(hydroxymethyl) tetrahydrofuran-2-yl)-5-(hydroxymethyl)-4-oxo-4,5-dihydro-3H-pyrazolo[4,3-d][1,2,3]triazine-7-carboxylate, 3-(3,4-dihydroxy-5-(hydroxymethyl)tetrahydrofuran-2-yl)-5-(hydroxymethyl)-7-propyl-3H-pyrazolo[4,3-d][1,2,3]triazin-4(5H)-one, 7-amino-3-(3,4-dihydroxy-5-(hydroxymethyl)tetrahydrofuran-2-yl)-5-(hydroxymethyl)-3H-pyrazolo[4,3-d][1,2,3]triazin-4(5H)-one, N-(3-(3,4-dihydroxy-5-(hydroxymethyl)tetrahydrofuran-2-yl)-5-(hydroxymethyl)-4-oxo-4,5-dihydro-3H-pyrazolo[4,3-d][1,2,3]triazin-7-yl)-3-(dimethylamino)propanamide, 3-(3,4-dihydroxy-5-(hydroxymethyl)tetrahydrofuran-2-yl)-5-(hydroxymethyl)-4-oxo-4,5-dihydro-3H-pyrazolo[4,3-d][1,2,3]triazine-7-carbonitrile, 3-(3,4-dihydroxy-5-(hydroxymethyl)tetrahydrofuran-2-yl)-5-(hydroxymethyl)-7-(piperidin-1-yl)-3H-pyrazolo[4,3-d][1,2,3]triazin-4(5H)-one, 3-(3,4-dihydroxy-5-(hydroxymethyl)tetrahydrofuran-2-yl)-5-(hydroxymethyl)-7-nitro-3H-pyrazolo[4,3-d][1,2,3]triazin-4(5H)-one, N-(3-(3,4-dihydroxy-5-(hydroxymethyl)tetrahydrofuran-2-yl)-5-(hydroxymethyl)-4-oxo-4,5-dihydro-3H-pyrazolo[4,3-d][1,2,3]triazin-7-yl)-4-methoxybenzamide, 2-((5-cyclopropyl-3-(3,4-dihydroxy-5-(hydroxymethyl)tetrahydro-furan-2-yl)-4-oxo-4,5-dihydro-3H-pyrazolo[4,3-d][1,2,3]triazin-7-yl)amino)-2-(diethoxyphosphoryl)acetic acid, ethyl 5-cyclopropyl-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate, 5-cyclopropyl-3-(3,4-dihydroxy-5-(hydroxymethyl)tetrahydrofuran-2-yl)-7-propyl-3H-pyrazolo[4,3-d][1,2,3]triazin-4(5H)-one, 7-amino-5-cyclopropyl-3-(3,4-dihydroxy-5-(hydroxymethyl) tetrahydrofuran-2-yl)-3H-pyrazolo[4,3-d][1,2,3]triazin-4(5H)-one, N-(5-cyclopropyl-3-(3,4-dihydroxy-5-(hydroxymethyl)tetrahydrofuran-2-yl)-4-oxo-4,5-dihydro-3H-pyrazolo[4,3-d][1,2,3]triazin-7-yl)-3-(dimethylamino)propanamide, 5-cyclopropyl-3-(3,4-dihydroxy-5-(hydroxymethyl)tetrahydrofuran-2-yl)-4-oxo-4,5-dihydro-3H-pyrazolo[4,3-d][1,2,3]triazine-7-carbonitrile, 5-cyclopropyl-3-(3,4-dihydroxy-5-(hydroxymethyl)tetrahydrofuran-2-yl)-7-(piperidin-1-yl)-3H-pyrazolo[4,3-d][1,2,3]triazin-4(5H)-one, 5-cyclopropyl-3-(3,4-dihydroxy-5-(hydroxymethyl)tetrahydrofuran-2-yl)-7-nitro-3H-pyrazolo[4,3-d][1,2,3]triazin-4(5H)-one, N-(5-cyclopropyl-3-(3,4-dihydroxy-5-(hydroxymethyl)tetrahydrofuran-2-yl)-4-oxo-4,5-dihydro-3H-pyrazolo[4,3-d][1,2,3]triazin-7-yl)-4-methoxybenzamide, 2-((5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazin-7-yl)(methyl)amino)-2-(diethoxyphosphoryl)acetic acid, ethyl 5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate, 2-(3-(3,4-dihydroxy-5-(hydroxymethyl)tetrahydrofuran-2-yl)-4-oxo-7-propyl-3H-pyrazolo [4,3-d][1,2,3]triazin-5(4H)-yl)acetamide, 2-(7-amino-3-(3,4-dihydroxy-5-(hydroxymethyl) tetrahydrofuran-2-yl)-4-oxo-3H-pyrazolo[4,3-d][1,2,3]triazin-5(4H)-yl)acetamide, N-(5-(2-amino-2-oxoethyl)-3-(3,4-dihydroxy-5-(hydroxymethyl)tetrahydrofuran-2-yl)-4-oxo-4,5-dihydro-3H-pyrazolo[4,3-d][1,2,3]triazin-7-yl)-3-(dimethylamino)propanamide, 2-(7-cyano-3-(3,4-dihydroxy-5-(hydroxymethyl) tetrahydrofuran-2-yl)-4-oxo-3H-pyrazolo[4,3-d][1,2,3]triazin-5(4H)-yl)acetamide, 2-(3-(3,4-dihydroxy-5-(hydroxymethyl)tetrahydrofuran-2-yl)-4-oxo-7-(piperidin-1-yl)-3H-pyrazolo[4,3-d][1,2,3]triazin-5(4H)-yl) acetamide, 2-(3-(3,4-dihydroxy-5-(hydroxymethyl)tetrahydrofuran-2-yl)-7-nitro-4-oxo-3H-pyrazolo[4,3-d][1,2,3]triazin-5(4H)-yl)acetamide, 3-(3,4-dihydroxy-5-(hydroxymethyl)tetrahydrofuran-2-yl)-4-oxo-7-propyl-3H-pyrazolo[4,3-d][1,2,3]triazine-5(4H)-carboxamide, 7-amino-3-(3,4-dihydroxy-5-(hydroxymethyl)tetrahydrofuran-2-yl)-4-oxo-3H-pyrazolo[4,3-d][1,2,3]triazine-5(4H)-carboxamide, 3-(3,4-dihydroxy-5-(hydroxymethyl)tetrahydrofuran-2-yl)-7-(3-(dimethylamino)propanamido)-4-oxo-3H-pyrazolo[4,3-d][1,2,3]triazine-5(4H)-carboxamide, 7-cyano-3-(3,4-dihydroxy-5-(hydroxymethyl) tetrahydrofuran-2-yl)-4-oxo-3H-pyrazolo[4,3-d][1,2,3]triazine-5(4H)-carboxamide, 3-(3,4-dihydroxy-5-(hydroxymethyl)tetrahydrofuran-2-yl)-4-oxo-7-(piperidin-1-yl)-3H-pyrazolo[4,3-d][1,2,3]triazine-5(4H)-carboxamide, 3-(3,4-dihydroxy-5-(hydroxymethyl)tetrahydrofuran-2-yl)-7-nitro-4-oxo-3H-pyrazolo[4,3-d][1,2,3]triazine-5(4H)-carboxamide, 3-(3,4-dihydroxy-5-(hydroxymethyl)tetrahydrofuran-2-yl)-7-(4-methoxybenzamido)-4-oxo-3H-pyrazolo[4,3-d][1,2,3]triazine-5(4H)-carboxamide, 2-(diethoxyphosphoryl)-2-(3-(3,4-dihydroxy-5-(hydroxymethyl)tetrahydrofuran-2-yl)-7-(ethoxycarbonyl)-4-oxo-3H-pyrazolo[4,3-d][1,2,3]triazin-5(4H)-yl)acetic acid, 2-(diethoxyphosphoryl)-2-(3-(3,4-dihydroxy-5-(hydroxymethyl) tetrahydrofuran-2-yl)-4-oxo-7-propyl-3H-pyrazolo[4,3-d][1,2,3]triazin-5(4H)-yl)acetic acid, 2-(7-cyano-3-(3,4-dihydroxy-5-(hydroxymethyl)tetrahydrofuran-2-yl)-4-oxo-3H-pyrazolo[4,3-d][1,2,3]triazin-5(4H)-yl)-2-(diethoxyphosphoryl)acetic acid, 2-(diethoxyphosphoryl)-2-(3-(3,4-dihydroxy-5-(hydroxymethyl) tetrahydrofuran-2-yl)-4-oxo-7-(piperidin-1-yl)-3H-pyrazolo[4,3-d][1,2,3]triazin-5(4H)-yl)acetic acid, 2-(diethoxyphosphoryl)-2-(3-(3,4-dihydroxy-5-(hydroxymethyl)tetrahydrofuran-2-yl)-7-nitro-4-oxo-3H-pyrazolo[4,3-d][1,2,3]triazin-5(4H)-yl)acetic acid, ethyl 5-carbamimidoyl-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate, 3-(3,4-dihydroxy-5-(hydroxymethyl)tetrahydrofuran-2-yl)-4-oxo-7-propyl-3H-pyrazolo[4,3-d][1,2,3]triazine-5(4H)-carboximidamide, 7-amino-3-(3,4-dihydroxy-5-(hydroxymethyl)tetrahydrofuran-2-yl)-4-oxo-3H-pyrazolo[4,3-d][1,2,3]triazine-5(4H)-carboximidamide, 7-cyano-3-(3,4-dihydroxy-5-(hydroxymethyl)tetrahydrofuran-2-yl)-4-oxo-3H-pyrazolo[4,3-d][1,2,3]triazine-5(4H)-carboximidamide, 3-(3,4-dihydroxy-5-(hydroxymethyl)tetrahydrofuran-2-yl)-4-oxo-7-(piperidin-1-yl)-3H-pyrazolo[4,3-d][1,2,3]triazine-5(4H)-carboximidamide, 3-(3,4-dihydroxy-5-(hydroxymethyl)tetrahydrofuran-2-yl)-7-nitro-4-oxo-3H-pyrazolo[4,3-d][1,2,3]triazine-5(4H)-carboximidamide, ethyl 5-carbamothioyl-3-(3,4-dihydroxy-5-(hydroxymethyl)tetrahydrofuran-2-yl)-4-oxo-4,5-dihydro-3H-pyrazolo[4,3-d][1,2,3]triazine-7-carboxylate, 3-(3,4-dihydroxy-5-(hydroxymethyl)tetrahydrofuran-2-yl)-4-oxo-7-propyl-3H-pyrazolo[4,3-d][1,2,3]triazine-5(4H)-carbothioamide, 7-amino-3-(3,4-dihydroxy-5-(hydroxymethyl)tetrahydrofuran-2-yl)-4-oxo-3H-pyrazolo[4,3-d][1,2,3]triazine-5(4H)-carbothioamide, N-(5-carbamothioyl-3-(3,4-dihydroxy-5-(hydroxymethyl)tetrahydrofuran-2-yl)-4-oxo-4,5-dihydro-3H-pyrazolo[4,3-d][1,2,3]triazin-7-yl)-3-(dimethylamino)propanamide, 7-cyano-3-(3,4-dihydroxy-5-(hydroxymethyl)tetrahydrofuran-2-yl)-4-oxo-3H-pyrazolo[4,3-d][1,2,3]triazine-5(4H)-carbothioamide, 3-(3,4-dihydroxy-5-(hydroxymethyl) tetrahydrofuran-2-yl)-7-nitro-4-oxo-3H-pyrazolo[4,3-d][1,2,3]triazine-5(4H)-carbothioamide, 5-hydroxy-3-(5-(hydroxymethyl)tetrahydrofuran-2-yl)-7-propyl-3H-pyrazolo[4,3-d][1,2,3]triazin-4(5H)-one, 3-(5-(hydroxymethyl)tetrahydrofuran-2-yl)-5-methyl-7-propyl-3H-pyrazolo[4,3-d][1,2,3]triazin-4(5H)-one, 2-(diethoxyphosphoryl)-2-((5-(hydroxymethyl)-3-(5-(hydroxymethyl)tetrahydrofuran-2-yl)-4-oxo-4,5-dihydro-3H-pyrazolo[4,3-d][1,2,3]triazin-7-yl)amino)acetic acid, 2-(diethoxyphosphoryl)-2-(3-(5-(hydroxymethyl)tetrahydrofuran-2-yl)-4-oxo-7-propyl-3H-pyrazolo[4,3-d][1,2,3]triazin-5(4H)-yl) acetic acid, 2-(7-amino-3-(5-(hydroxymethyl)tetrahydrofuran-2-yl)-4-oxo-3H-pyrazolo[4,3-d][1,2,3]triazin-5(4H)-yl)-2-(diethoxyphosphoryl)acetic acid, 2-(7-cyano-3-(5-(hydroxymethyl)tetrahydrofuran-2-yl)-4-oxo-3H-pyrazolo[4,3-d][1,2,3]triazin-5(4H)-yl)-2-(diethoxyphosphoryl)acetic acid, 3-(5-(hydroxymethyl)tetrahydrofuran-2-yl)-4-oxo-7-propyl-3H-pyrazolo[4,3-d][1,2,3]triazine-5(4H)-carboximidamide, 3-(5-(hydroxymethyl)tetrahydrofuran-2-yl)-4-oxo-7-propyl-3H-pyrazolo[4,3-d][1,2,3]triazine-5(4H)-carbothioamide, 2-(diethoxyphosphoryl)-2-((5-hydroxy-3-(2-(hydroxymethyl)-1,3-oxathiolan-5-yl)-4-oxo-4,5-dihydro-3H-pyrazolo[4,3-d][1,2,3]triazin-7-yl)amino)acetic acid, 5-hydroxy-3-(2-(hydroxymethyl)-1,3-oxathiolan-5-yl)-7-propyl-3H-pyrazolo[4,3-d][1,2,3]triazin-4(5H)-one, 7-amino-5-hydroxy-3-(2-(hydroxymethyl)-1,3-oxathiolan-5-yl)-3H-pyrazolo[4,3-d][1,2,3]triazin-4(5H)-one, 2-(diethoxyphosphoryl)-2-((3-(2-(hydroxymethyl)-1,3-oxathiolan-5-yl)-5-methyl-4-oxo-4,5-dihydro-3H-pyrazolo[4,3-d][1,2,3]triazin-7-yl)amino)acetic acid, 3-(2-(hydroxymethyl)-1,3-oxathiolan-5-yl)-5-methyl-7-propyl-3H-pyrazolo[4,3-d][1,2,3]triazin-4(5H)-one, 3-(2-(hydroxymethyl)-1,3-oxathiolan-5-yl)-5-methyl-4-oxo-4,5-dihydro-3H-pyrazolo[4,3-d][1,2,3]triazine-7-carbonitrile, 3-(2-(hydroxymethyl)-1,3-oxathiolan-5-yl)-5-methyl-7-nitro-3H-pyrazolo[4,3-d][1,2,3]triazin-4(5H)-one, 5-(hydroxymethyl)-3-(2-(hydroxymethyl)-1,3-oxathiolan-5-yl)-7-propyl-3H-pyrazolo[4,3-d][1,2,3]triazin-4(5H)-one, 2-(3-(2-(hydroxymethyl)-1,3-oxathiolan-5-yl)-4-oxo-7-propyl-3H-pyrazolo[4,3-d][1,2,3]triazin-5(4H)-yl) acetamide, 2-(7-amino-3-(2-(hydroxymethyl)-1,3-oxathiolan-5-yl)-4-oxo-3H-pyrazolo[4,3-d][1,2,3]triazin-5(4H)-yl)acetamide, 3-(2-(hydroxymethyl)-1,3-oxathiolan-5-yl)-4-oxo-7-propyl-3H-pyrazolo[4,3-d][1,2,3]triazine-5(4H)-carboxamide, 7-amino-3-(2-(hydroxymethyl)-1,3-oxathiolan-5-yl)-4-oxo-3H-pyrazolo[4,3-d][1,2,3]triazine-5(4H)-carboxamide, 7-(3-(dimethylamino)propanamido)-3-(2-(hydroxymethyl)-1,3-oxathiolan-5-yl)-4-oxo-3H-pyrazolo[4,3-d][1,2,3]triazine-5(4H)-carboxamide, 3-(2-(hydroxymethyl)-1,3-oxathiolan-5-yl)-4-oxo-7-propyl-3H-pyrazolo[4,3-d][1,2,3]triazine-5(4H)-carboximidamide, 3-(2-(hydroxymethyl)-1,3-oxathiolan-5-yl)-4-oxo-7-propyl-3H-pyrazolo[4,3-d][1,2,3]triazine-5(4H)-carbothioamide, 2-(bis(isopropylamino)phosphoryl)-2-((5-(7-(ethoxycarbonyl)-5-hydroxy-4-oxo-4,5-dihydro-3H-pyrazolo[4,3-d][1,2,3]triazin-3-yl)-3,4-dihydroxytetrahydrofuran-2-yl)methoxy) acetic acid, 2-(bis(isopropylamino)phosphoryl)-2-((3,4-dihydroxy-5-(5-hydroxy-4-oxo-7-propyl-4,5-dihydro-3H-pyrazolo[4,3-d][1,2,3]triazin-3-yl)tetrahydrofuran-2-yl)methoxy)acetic acid, 2-((5-(7-amino-5-hydroxy-4-oxo-4,5-dihydro-3H-pyrazolo[4,3-d][1,2,3]triazin-3-yl)-3,4-dihydroxytetrahydrofuran-2-yl) methoxy)-2-(bis(isopropylamino)phosphoryl)acetic acid, 2-(bis(isopropylamino)phosphoryl)-2-((5-(7-(ethoxycarbonyl)-5-methyl-4-oxo-4,5-dihydro-3H-pyrazolo[4,3-d][1,2,3]triazin-3-yl)-3,4-dihydroxytetrahydrofuran-2-yl)methoxy)acetic acid, 2-(bis(isopropylamino)phosphoryl)-2-((3,4-dihydroxy-5-(5-methyl-4-oxo-7-propyl-4,5-dihydro-3H-pyrazolo[4,3-d][1,2,3]triazin-3-yl)tetrahydrofuran-2-yl)methoxy)acetic acid, 2-((5-(7-amino-5-methyl-4-oxo-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid, 2-(bis(isopropylamino)phosphoryl)-2-((5-(7-cyano-5-methyl-4-oxo-4,5-dihydro-3H-pyrazolo[4,3-d][1,2,3]triazin-3-yl)-3,4-dihydroxytetrahydrofuran-2-yl)methoxy)acetic acid, 2-(bis(isopropylamino)phosphoryl)-2-((5-(7-(ethoxycarbonyl)-5-(hydroxymethyl)-4-oxo-4,5-dihydro-3H-pyrazolo[4,3-d][1,2,3]triazin-3-yl)-3,4-dihydroxytetrahydrofuran-2-yl)methoxy)acetic acid, 2-(bis(isopropylamino)phosphoryl)-2-((3,4-dihydroxy-5-(5-(hydroxymethyl)-4-oxo-7-propyl-4,5-dihydro-3H-pyrazolo[4,3-d][1,2,3]triazin-3-yl) tetrahydrofuran-2-yl)methoxy)acetic acid, 2-(bis(isopropylamino)phosphoryl)-2-((5-(5-cyclopropyl-4-oxo-7-propyl-4,5-dihydro-3H-pyrazolo[4,3-d][1,2,3]triazin-3-yl)-3,4-dihydroxytetrahydrofuran-2-yl) methoxy)acetic acid, 2-((5-(5-(2-amino-2-oxoethyl)-4-oxo-7-propyl-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid, 2-((5-(7-amino-5-(2-amino-2-oxoethyl)-4-oxo-4,5-dihydro-3H-pyrazolo[4,3-d][1,2,3]triazin-3-yl)-3,4-dihydroxytetrahydrofuran-2-yl)methoxy)-2-(bis(isopropylamino)phosphoryl)acetic acid, 2-(bis(isopropylamino)phosphoryl)-2-((5-(5-carbamoyl-4-oxo-7-propyl-4,5-dihydro-3H-pyrazolo[4,3-d][1,2,3]triazin-3-yl)-3,4-dihydroxytetrahydrofuran-2-yl)methoxy)acetic acid, 2-(bis(isopropylamino)phosphoryl)-2-(((5-(5-carbamoyl-7-(3-(dimethylamino)propanamido)-4-oxo-4,5-dihydro-3H-pyrazolo[4,3-d][1,2,3]triazin-3-yl)-3,4-dihydroxytetrahydrofuran-2-yl)methyl)amino)acetic acid, 2-((cyanomethoxy) (isopropylamino)phosphino)-2-((5-(7-(ethoxycarbonyl)-5-hydroxy-4-oxo-4,5-dihydro-3H-pyrazolo[4,3-d][1,2,3]triazin-3-yl)-3,4-dihydroxytetrahydrofuran-2-yl)methoxy)acetic acid, 2-((cyanomethoxy) (isopropylamino)phosphino)-2-((3,4-dihydroxy-5-(5-hydroxy-4-oxo-7-propyl-4,5-dihydro-3H-pyrazolo[4,3-d][1,2,3]triazin-3-yl)tetrahydrofuran-2-yl)methoxy)acetic acid, 2-((5-(7-cyano-5-hydroxy-4-oxo-4,5-dihydro-3H-pyrazolo[4,3-d][1,2,3]triazin-3-yl)-3,4-dihydroxytetrahydrofuran-2-yl)methoxy)-2-((cyanomethoxy)(isopropylamino)phosphino)acetic acid, 2-((cyanomethoxy)(isopropylamino)phosphino)-2-((5-(7-(ethoxycarbonyl)-5-methyl-4-oxo-4,5-dihydro-3H-pyrazolo[4,3-d][1,2,3]triazin-3-yl)-3,4-dihydroxytetrahydrofuran-2-yl)methoxy)acetic acid, 2-((cyanomethoxy)(isopropylamino)phosphino)-2-((3,4-dihydroxy-5-(5-methyl-4-oxo-7-propyl-4,5-dihydro-3H-pyrazolo[4,3-d][1,2,3]triazin-3-yl) tetrahydrofuran-2-yl)methoxy)acetic acid, 2-((5-(5-(2-amino-2-oxoethyl)-7-(ethoxycarbonyl)-4-oxo-4,5-dihydro-3H-pyrazolo[4,3-d][1,2,3]triazin-3-yl)-3,4-dihydroxytetrahydrofuran-2-yl)methoxy)-2-((cyanomethoxy)(isopropylamino)phosphino)acetic acid, 2-((cyanomethoxy)(isopropylamino)phosphino)-2-((5-(5-cyclopropyl-4-oxo-7-propyl-4,5-dihydro-3H-pyrazolo[4,3-d][1,2,3]triazin-3-yl)-3,4-dihydroxytetrahydrofuran-2-yl)methoxy)acetic acid, 2-((5-(5-carbamoyl-7-cyano-4-oxo-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid, 2-((5-(5-carbamimidoyl-4-oxo-7-propyl-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid, 2-((5-(5-carbamothioyl-4-oxo-7-propyl-4,5-dihydro-3H-pyrazolo[4,3-d][1,2,3]triazin-3-yl)-3,4-dihydroxytetrahydrofuran-2-yl)methoxy)-2-((cyanomethoxy) (isopropylamino)phosphino)acetic acid, 3-(((1,3-dihydroxypropan-2-yl)oxy)methyl)-5-methyl-7-propyl-3H-pyrazolo[4,3-d][1,2,3]triazin-4(5H)-one, 2-(diethoxyphosphoryl)-2-((3-(((1,3-dihydroxypropan-2-yl) oxy)methyl)-5-(hydroxymethyl)-4-oxo-4,5-dihydro-3H-pyrazolo[4,3-d][1,2,3]triazin-7-yl)amino)acetic acid, 2-((5-(2-amino-2-oxoethyl)-3-(((1,3-dihydroxypropan-2-yl)oxy)methyl)-4-oxo-4,5-dihydro-3H-pyrazolo[4,3-d][1,2,3]triazin-7-yl)amino)-2-(diethoxyphosphoryl)acetic acid, N-(5-(2-amino-2-oxoethyl)-3-(((1,3-dihydroxypropan-2-yl)oxy)methyl)-4-oxo-4,5-dihydro-3H-pyrazolo[4,3-d][1,2,3]triazin-7-yl)-3-(dimethylamino)propanamide, 3-(((1,3-dihydroxypropan-2-yl)oxy)methyl)-4-oxo-7-propyl-3H-pyrazolo [4,3-d][1,2,3]triazine-5(4H)-carboximidamide, isopropyl 2-((((5-(5-carbamimidoyl-4-oxo-7-propyl-4,5-dihydro-3H-pyrazolo[4,3-d][1,2,3]triazin-3-yl)tetrahydrofuran-2-yl)methoxy)(phenoxy)phosphorothioyl) amino)propanoate, ethyl ethyl 3-(3,4-dihydroxy-5-(((hydroxy((hydroxy(phosphonooxy)phosphoryl)oxy) phosphoryl)oxy)methyl)tetrahydrofuran-2-yl)-5-hydroxy-4-oxo-4,5-dihydro-3H-pyrazolo[4,3-d][1,2,3]triazine-7-carboxylate, (3,4-dihydroxy-5-(5-hydroxy-4-oxo-7-propyl-4,5-dihydro-3H-pyrazolo[4,3-d][1,2,3]triazin-3-yl)tetrahydrofuran-2-yl)methyl tetrahydrogen triphosphate, (5-(7-cyano-5-hydroxy-4-oxo-4,5-dihydro-3H-pyrazolo[4,3-d][1,2,3]triazin-3-yl)-3,4-dihydroxytetrahydrofuran-2-yl)methyl tetrahydrogentriphosphate, (3,4-dihydroxy-5-(5-methyl-4-oxo-7-propyl-4,5-dihydro-3H-pyrazolo[4,3-d][1,2,3]triazin-3-yl)tetrahydrofuran-2-yl)methyl tetrahydrogen triphosphate, (5-(7-amino-5-methyl-4-oxo-4,5-dihydro-3H-pyrazolo[4,3-d][1,2,3]triazin-3-yl)-3,4-dihydroxytetrahydrofuran-2-yl)methyl tetrahydrogen triphosphate, (5-(7-cyano-5-methyl-4-oxo-4,5-dihydro-3H-pyrazolo[4,3-d][1,2,3]triazin-3-yl)-3,4-dihydroxytetrahydrofuran-2-yl)methyl tetrahydrogen triphosphate, (3,4-dihydroxy-5-(5-methyl-7-nitro-4-oxo-4,5-dihydro-3H-pyrazolo[4,3-d][1,2,3]triazin-3-yl)tetrahydrofuran-2-yl)methyl tetrahydrogen triphosphate, (5-(5-carbamoyl-4-oxo-7-propyl-4,5-dihydro-3H-pyrazolo[4,3-d][1,2,3]triazin-3-yl)-3,4-dihydroxytetrahydrofuran-2-yl)methyl tetrahydrogen triphosphate, (5-(5-carbamimidoyl-4-oxo-7-propyl-4,5-dihydro-3H-pyrazolo[4,3-d][1,2,3]triazin-3-yl)-3,4-dihydroxytetrahydrofuran-2-yl) methytetrahydrogen triphosphate, (5-(5-carbamothioyl-4-oxo-7-propyl-4,5-dihydro-3H-pyrazolo[4,3-d][1,2,3]triazin-3-yl)-3,4-dihydroxytetrahydrofuran-2-yl)methyl tetrahydrogen triphosphate, ethyl 3-(4-(((2-cyanoethoxy)(diisopropylamino)phosphino)oxy)-5-(hydroxymethyl)tetrahydrofuran-2-yl)-5-hydroxy-4-oxo-4,5-dihydro-3H-pyrazolo[4,3-d][1,2,3]triazine-7-carboxylate, 5-(7-amino-5-hydroxy-4-oxo-4,5-dihydro-3H-pyrazolo[4,3-d][1,2,3]triazin-3-yl)-2-(hydroxymethyl)tetrahydrofuran-3-yl (2-cyanoethyl) diisopropylphosphoramidite, 2-cyanoethyl (2-(hydroxymethyl)-5-(5-methyl-4-oxo-7-propyl-4,5-dihydro-3H-pyrazolo[4,3-d][1,2,3]triazin-3-yl)tetrahydrofuran-3-yl) diisopropylphosphoramidite, 2-cyanoethyl (2-(hydroxymethyl)-5-(5-(hydroxymethyl)-4-oxo-7-propyl-4,5-dihydro-3H-pyrazolo[4,3-d][1,2,3]triazin-3-yl)tetrahydrofuran-3-yl) diisopropylphosphoramidite, 2-cyanoethyl (2-(hydroxymethyl)-5-(5-(hydroxymethyl)-7-nitro-4-oxo-4,5-dihydro-3H-pyrazolo[4,3-d][1,2,3]triazin-3-yl) tetrahydrofuran-3-yl) diisopropylphosphoramidite, 6-(3,4-dihydroxy-5-(hydroxymethyl)tetrahydrofuran-2-yl)-1-propyl-1 H-[1,2,3]triazolo[4,5-d][1,2,3]triazin-7(6H)-one, 6-(3,4-dihydroxy-5-(hydroxymethyl) tetrahydrofuran-2-yl)-1-(hydroxymethyl)-1H-[1,2,3]triazolo[4,5-d][1,2,3]triazin-7(6H)-one, ethyl 2-(6-(3,4-dihydroxy-5-(hydroxymethyl)tetrahydrofuran-2-yl)-7-oxo-6,7-dihydro-H-[1,2,3]triazolo[4,5-d][1,2,3] triazin-1-yl)acetate, 2-(bis(isopropylamino)phosphoryl)-2-((5-(7-oxo-1-propyl-H-[1,2,3]triazolo[4,5-d][1,2,3]triazin-6(7H)-yl)tetrahydrofuran-2-yl)methoxy)acetic acid, 6-(3,4-dihydroxy-5-(hydroxymethyl) tetrahydrofuran-2-yl)-3-propyl-3H-[1,2,3]triazolo[4,5-d][1,2,3]triazin-7(6H)-one, 6-(3,4-dihydroxy-5-(hydroxymethyl)tetrahydrofuran-2-yl)-3-(hydroxymethyl)-3H-[1,2,3]triazolo[4,5-d][1,2,3]triazin-7(6H)-one, ethyl 2-(6-(3,4-dihydroxy-5-(hydroxymethyl)tetrahydrofuran-2-yl)-7-oxo-6,7-dihydro-3H-[1,2,3]triazolo[4,5-d][1,2,3]triazin-3-yl)acetate, 2-(bis(isopropylamino)phosphoryl)-2-((5-(7-oxo-3-propyl-3H-[1,2,3]triazolo[4,5-d][1,2,3]triazin-6(7H)-yl)tetrahydrofuran-2-yl)methoxy)acetic acid, 7-cyclopropyl-3-(3-hydroxy-4-(hydroxymethyl)oxetan-2-yl)-3H-pyrazolo[3,4-d][1,2,3]triazin-4(7H)-one, 7-cyclopropyl-3-(3-(hydroxymethyl)oxiran-2-yl)-3H-pyrazolo[3,4-d][1,2,3]triazin-4(7H)-one, 2-(diethoxyphosphoryl)-2-((5-(hydroxymethyl)-4-oxo-3-(3,4,5-trihydroxy-6-(hydroxymethyl) tetrahydro-2H-pyran-2-yl)-4,5-dihydro-3H-pyrazolo[4,3-d][1,2,3]triazin-7-yl)amino)acetic acid, 3-(hydroxymethyl)-5-(3,4,5-trihydroxy-6-(hydroxymethyl)tetrahydro-2H-pyran-2-yl)-3H-[1,2,3]triazolo[4,5-d][1,2,3]triazin-4(5H)-one, 2-(diethoxyphosphoryl)-2-(4-oxo-7-propyl-3-(3,4,5-trihydroxy-6-(hydroxymethyl)tetrahydro-2H-pyran-2-yl)-3H-pyrazolo[4,3-d][1,2,3]triazin-5(4H)-yl) acetic acid, 5-(hydroxymethyl)-7-propyl-3-(3,4,5-trihydroxy-6-(hydroxymethyl)tetrahydro-2H-pyran-2-yl)-3H-pyrazolo[4,3-d][1,2,3]triazin-4(5H)-one, 1-methyl-3-propyl-5-(3,4,5-trihydroxy-6-(hydroxymethyl)tetrahydro-2H-pyran-2-yl)-1H-pyrazolo[3,4-d][1,2,3]triazin-4(5H)-one, 1-(hydroxymethyl)-3-propyl-5-(3,4,5-trihydroxy-6-(hydroxymethyl)tetrahydro-2H-pyran-2-yl)-1H-pyrazolo[3,4-d][1,2,3]triazin-4(5H)-one, 4-methoxy-N-(5-methyl-4-oxo-3-(3,4,5-trihydroxy-6-(hydroxymethyl)tetrahydro-2H-pyran-2-yl)-4,5-dihydro-3H-pyrazolo[4,3-d][1,2,3]triazin-7-yl) benzamide, 3-(dimethylamino)-N-(5-methyl-4-oxo-3-(3,4,5-trihydroxy-6-(hydroxymethyl)tetrahydro-2H-pyran-2-yl)-4,5-dihydro-3H-pyrazolo[4,3-d][1,2,3]triazin-7-yl)propanamide, 1-(hydroxymethyl)-5-(3,4,5-trihydroxy-6-(hydroxymethyl)tetrahydro-2H-pyran-2-yl)-1H-[1,2,3]triazolo[4,5-d][1,2,3]triazin-4(5H)-one, ethyl 7-carbamoyl-1-oxo-2-(3,4,5-trihydroxy-6-(hydroxymethyl)tetrahydro-2H-pyran-2-yl)-2,7-dihydro-1H-pyrazolo[4,3-d][1,2,3]triazine-5-carboxylate, 5-methyl-7-propyl-3-(3,4,5-trihydroxy-6-(hydroxymethyl)tetrahydro-2H-pyran-2-yl)-3H-pyrazolo[4,3-d][1,2,3]triazin-4(5H)-one.
